(19) United States
(12) Patent Application Publication
(10) Pub. No.: US 2022/0372475 A1
(43) Pub. Date: Nov. 24, 2022

(54) Inhibitors Of RNA Editing And Uses Thereof

(71) Applicants: National University of Singapore, Singapore (SG); Nanyang Technological University, Singapore (SG)

(72) Inventors: Leilei Chen, Singapore (SG); Daryl JT Tay, Singapore (SG); Gang Chen, Shenzhen (CN)

(21) Appl. No.: 17/623,863

(22) PCT Filed: Jul. 3, 2020
(86) PCT No.: PCT/SG2020/050380

§ 371 Date: Dec. 29, 2021

(30) Foreign Application Priority Data
Jul. 4, 2019 (SG) 10201906239R

(51) Int. Cl. C12N 15/113; A61P 35/00

(52) U.S. Cl. CPC C12N 15/113; A61P 35/00; C12N 2310/11; C12N 2320/30; C12N 2310/321

(57) ABSTRACT

The present invention provided an oligonucleotide targeting the core editing-site complementary sequence (ECS) of AZIN1 gene, wherein the core ECS of AZIN1 gene comprises the sequence 5′-GCTTTTCC-3′, and wherein the oligonucleotide comprises one or more nucleotides with sugar modification and one or more modified internucleotide linkages. In another aspect, there is provided a pharmaceutical composition comprising the oligonucleotide as disclosed herein. In another aspect, there is provided a method of inhibiting AZIN1 pre-mRNA editing in a cell, wherein the AZIN1 pre-mRNA editing is mediated by adenosine deaminase acting on RNA-1 (ADAR-1), as well as a method of using the same for the treatment of cancers associated with AZIN1 pre-mRNA editing, including liver cancer.

### CROSS-REFERENCE TO RELATED APPLICATIONS

This application claims the benefit of priority of the Singapore application No. 10201906239R, filed on 4 Jul. 2019, the contents of it being hereby incorporated by reference in its entirety for all purposes.

### FIELD OF THE INVENTION

The present invention generally relates to the fields of molecular biology, cell biology and biotechnology. More particularly, the present invention relates to oligonucleotides for inhibition of RNA editing, compositions comprising the oligonucleotides, and uses of the oligonucleotides and compositions.

### BACKGROUND OF THE INVENTION

Cancer generally refers to a group of diseases involving abnormal cell growth with the potential to invade or spread to other parts of the body. Cancer has a high prevalence around the world, with estimates as high as 90.5 million people in 2015. The Centre for Disease Control (CDC) expects that between the years of 2010 and 2020, the number of new cancer cases in the United States may go up about 24% in men to more than 1 million cases per year, and by about 21% in women to more than 900,000 cases per year. The kinds of cancer that are expected to increase the most are: melanoma in white men and women, prostate, kidney, liver, and bladder cancers in men, and lung, breast, uterine, and thyroid cancers in women.

RNA editing is a widespread process which introduces changes in RNA sequences encoded by the genome, contributing to “RNA mutations”. Aberrant RNA editing of specific genes and their association with cancer progression have been discovered in many cancer types in the past decade, including but not limited to hepatocellular carcinoma (HCC), esophageal squamous cell carcinoma (ESCC), non-small cell lung cancer (NSCLC), and colorectal cancer (CRC).

Adenosine deaminase acting on RNA (ADAR) is an enzyme that in humans is encoded by the ADAR gene. ADAR is a RNA-binding protein, which functions in RNA-editing through post-transcriptional modification of mRNA transcripts by changing the nucleotide content of the RNA. ADARs are responsible for binding to double stranded RNA (dsRNA) and converting adenosine (A) to inosine (I) by deamination. Inosine is structurally similar to that of guanine (G) which leads to I to cytosine (C) binding. Inosine typically mimicks guanosine during translation. Thus, the conversion from A to I in the RNA disrupts the normal A:U pairing which makes the RNA unstable. Codon changes can also arise from editing which may lead to changes in the coding sequences for proteins and their functions. ADAR also impacts the transcriptome in editing-independent ways, likely by interfering with other RNA-binding proteins.

In mammals, there are three types of ADARs, ADAR1, ADAR2 and ADAR3. ADAR1 and ADAR2 are found in many tissues in the body while ADAR3 is only found in the brain. Studies have shown that ADAR1 and ADAR2 are frequently dysregulated in cancers. It has been suggested that ADAR1 is responsible for the disrupted A to I editing pattern seen in various cancers. The dysregulation of ADAR1 expression could change the frequency of A to I transitions in the protein coding region of oncogenes or tumor suppressor genes, resulting in mutated oncogene or tumor suppressor gene products which drive the development of cancers.

As the ADAR proteins have thousands of editing substrates, simply modulating the expression of ADARs may cause considerable off-target effects. Therefore, there is a need for ADAR inhibitors that specifically inhibit the RNA edition of the oncogenes or tumor suppressor genes targeted by ADARs.

### SUMMARY OF THE INVENTION

In one aspect of the present invention, there is provided an oligonucleotide targeting the core editing-site complementary sequence (ECS) of AZIN1 gene, wherein the core ECS of AZIN1 gene comprises the sequence 5′-GCTTTTCC-3′, and wherein the oligonucleotide comprises one or more nucleotides with sugar modification and one or more modified internucleotide linkages. In another aspect, there is provided a pharmaceutical composition comprising the oligonucleotide as disclosed herein. In another aspect, there is provided a method of inhibiting AZIN1 pre-mRNA editing in a cell, the method comprising contacting the cell with the oligonucleotide as disclosed herein, or the pharmaceutical composition as disclosed herein. In another aspect, there is provided a method of treating cancer in a subject in need thereof, comprising administering to the subject a therapeutically effective amount of the oligonucleotide as disclosed herein, or the pharmaceutical composition as disclosed herein, wherein the cancer is associated with AZIN1 pre-mRNA editing.

### BRIEF DESCRIPTION OF THE DRAWINGS

The invention will be better understood with reference to the detailed description when considered in conjunction with the non-limiting examples and the accompanying drawings, in which:

FIG. 1 shows that the 3′end sequence of exon 12 is required for AZIN1 editing. FIG. 1A is a schematic diagram of AZIN1 minigene constructs generated by inserting 5 different fragments (FA, FB, FC, FD or FE) covering the edited exon 11 and flanking exons (exon 10 and 12) and introns (intron 9, 10, 11 and 12) into either pRK7 or pcDNA3.1 vector. The arrow indicates relative position of the editing site. FIGS. 1B and 1C are sequencing chromatograms illustrating editing of endogenous AZIN1 (FIG. 1B, left panel) and exogenous HTR2C (FIG. 1B, right panel) or AZIN1 (FIG. 1C) transcripts transcribed from pRK7-based minigene constructs in the HEK293T cells co-transfected with the indicated pRK7 minigenes and empty vector (EV) or ADAR1 expression construct (ADAR1). FIG. 1D shows sequencing chromatograms illustrating editing of endogenous and exogenous AZIN1 transcripts in HEK293T cells co-transfected with pcDNA3.1-based minigenes and EV or ADAR1. In FIG. 1B-1D, percentage of editing is calculated as area of “G” peak over the total area of “A” and “G” peaks. * indicates no editing is detected. Black arrow indicates the position of editing site. FIG. 1E shows predicted RNA second structure of AZIN1 by RNAfold. An 8-nt sequence indicated by the small arrows on the right are the potential core ECS. Editing site is indicated by the solid arrowhead on the left. Minimum free energy (MFE) structures drawing encoding base-pair probabilities are shown. Base-pair probabilities are shown by a colour spectrum. Taken together, FIG. 1 shows that amongst all AZIN1 minigenes (using either pRK7-based or pcDNA3.1-based minigene systems), only AZIN1 transcripts transcribed from the minigene containing fragment A (FA), which lacks 90-bp sequence at 3′ end of exon 12, was unable to be edited. This suggests that the ECS of AZIN1 is located at the 3′ end of exon 12.

FIG. 2 shows that an 8-nt sequence at 3′end of exon 12 is the core ECS and indispensable for AZIN1 editing. FIG. 2A is a schematic diagram of FE-1, 2 and 3 minigene constructs. Small arrow heads at the bottom indicate the mutations introduced into FE-3 minigene. Big arrow on top indicates relative position of the editing site. FIG. 2B shows predicted RNA secondary structure of AZIN1 transcript transcribed from the indicated minigene by RNAfold. Black arrow indicates the editing site. MFE structures drawing encoding base-pair probabilities are shown. Base-pair probabilities are shown by a colour spectrum. FIG. 2C shows sequencing chromatograms illustrating editing of endogenous and exogenous AZIN1 transcripts in HEK293T cells co-transfected with pRK7-based minigenes and EV or ADAR1. FIG. 2D shows results of in vitro RNA editing analysis of AZIN1 transcripts. FIG. 2D left panel: In vitro transcribed HTR2C or AZIN1 transcripts from the indicated minigene construct were incubated with purified ADAR1 protein, followed by RNA editing analysis using Sanger sequencing. In vitro transcribed HTR2C serves as a positive control. FIG. 2D Right panel: Data is presented in the bar chart as the mean±s.d. of three technical triplicates from a representative experiment. n.d., not detectable. In FIGS. 2C and 2D, percentage of editing is calculated as area of “G” peak over the total area of “A” and “G” peaks. *, no editing detected. Black arrow indicates the position of editing site. Taken together, FIG. 2 shows that transcripts transcribed from FE-1 (deleting 29-bp sequence at 3′ end of exon 12, FE-2 (deleting 8-bp sequence near the 3′ end of exon 12), and FE-3 (point mutations near the 3′ end of exon 12) minigenes failed to be edited upon ADAR1 overexpression, indicating that an 8-nt sequence (5′-GCUUUUCC-3′) at the 3′end of exon 12 is the core ECS of AZIN1 editing.

FIG. 3 shows screening of effective antisense oligonucleotides (ASOs) that can bind to AZIN1 duplex and inhibit AZIN1 editing in vitro. FIG. 3A shows illustration of the design of ASOs. A short RNA duplex containing partial exon 11 with the adenosine that undergoes deamination (editing site, solid underline), and a partial exon 12 sequence containing the ECS with the core 8-nt ECS (ECS region, dashed underline), is used for designing ASOs that target either the editing or ECS region. ASP1, DSP1, and DSP2 are peptide nucleic acid (PNA), whereas ASOs 1-7 are ASOs using canonical bases that are 2′-O-Me modified. Sequence of each oligo and their characteristics are listed in Table 3. FIG. 3B shows results of REMSA performed to examine the binding of each ASO (2.5 μM) to 32P-labelled AZIN1 RNA duplexes (86-nt). The sequence and predicted structure of the duplex probe are provided in Table 2 and FIG. 9A. Vehicle control (VC) means no ASO added. FIG. 3C shows binding of ASO1, 3, 5 or 7 to 32P-labelled AZIN1 RNA duplexes detected by REMSA, at different concentrations as indicated. FIG. 3D shows in vitro RNA editing analysis of AZIN1 transcripts transcribed from the FE minigene, after the incubation with purified ADAR1 protein and 200 nM of the indicated ASO. FIG. 3D top panel: sequencing chromatograms illustrating editing of in vitro transcribed AZIN1 transcripts in the indicated samples. Percentage of editing is calculated as area of “G” peak over the total area of “A” and “G” peaks. Arrow indicates the position of editing site. *, no editing detected. FIG. 3D bottom panel: data is presented in the bar chart as the mean±s.d. of three independent experiments. The value shown on the top of each bar is the mean value. n.d., not detectable. Taken together, FIG. 3 shows that ASO1, ASO3, ASO5 and ASO7 can bind to the AZIN1 dsRNA in a dose-dependent manner, ASO1, ASO3 can completely inhibit AZIN1 editing in vitro, and that ASO5 can substantially inhibit AZIN1 editing in vitro.

FIG. 4 shows that ECS-targeting ASOs abolish or inhibit AZIN1 editing in cancer cells. FIG. 4A shows illustration of chemical modifications of ASOs. Fully 2′-O-Me-modified ASO1 and ASO3 were further fully or partially modified with phosphorothioate (PS) bonds, indicated with asterisks (see also Table 3). FIG. 4B shows results of semi-quantitative PCR analysis of AZIN1 transcripts in KYSE510 and H358 cells treated with 100 nM of each of the indicated ASOs. Agarose gel electrophoresis of PCR amplicons showing two isoforms of AZIN1. The fast-moving band indicates an exon 11-skipping isoform of AZIN1. Sanger sequencing chromatograms data showing the junction between exon 10 and exon 12 can be seen in FIG. 10B. The results in FIG. 4B show that seven ASOs (ASO1, 1.1, 1.2, 1.3, 5, 6, and 7) targeting the editing region led to skipping of exon 11. FIG. 4C shows results of in silico prediction of splicing factor binding sites on the editing region of AZIN1 pre-mRNA by SpliceAid231. SRSF3, SRSF6, and SRSF1 are predicted to bind to the editing region. Editing site is underlined. FIG. 4D shows results of QPCR analysis of AZIN1 expression in KYSE510 cells treated with 100 nM of each of the indicated ASOs. Data are presented as the mean±s.d. of triplicates from a representative experiment. FIG. 4E shows results of Western blot analysis of AZIN1 and ADAR1 protein expression in KYSE510 cells treated with 100 nM of each of the indicated ASOs. Approximately 20 μg of protein lysate extracted from HEK293T cells transfected with AZIN1 expression construct was included as a positive control for the AZIN1 protein. GAPDH was used as a loading control. FIGS. 4F and 4G are sequencing chromatograms show editing of AZIN1 transcripts in KYSE510 cells treated with 100 nM of each of the indicated ASOs. Percentage of editing is calculated as area of “G” peak over the total area of “A” and “G” peaks. Arrow indicates the position of editing site. *, no editing detected. Data is presented in the bar chart (G) as the mean±s.d. of three technical triplicates from a representative experiment. The value shown on the top of each bar is the mean value. n.d., not detectable. Taken together, FIGS. 4D-4G show that of the three ECS-targeting ASOs, ASO3.1 and ASO3.2 completely abolished AZIN1 editing, and ASO3.3 dramatically inhibited editing, without affecting splicing and expression of AZIN1 at both mRNA and protein levels.

FIG. 5 shows ASO3.2 specifically inhibits GUS transition and cancer cell viability. FIG. 5A shows cell viability of KYSE510 (K510), H358, or KYSE180 (K180) cells measured by CellTiter-Glo® (CTG) assays, after the treatment with different concentrations (1, 10, 25, 50, 100, 150, 200, and 250 nM) of ASO3.1, ASO 3.2 or ASO-ctl for 48 hours. The corresponding half maximal inhibitory concentration (IC50) values are shown for each cell line. Data are presented as the mean±s.d. of four replicates from a representative experiment. The results in FIG. 5A show that both ASO3.1 and ASO3.2 dramatically inhibited cell viability of KYSE510 and H358 with low IC50 values, while they demonstrated much less inhibitory effects on cell viability of KYSE180. FIG. 5B shows cell viability of each of three cancer cell lines and normal hepatocytes measured by CTG assays, after the treatment with 50 nM of ASO3.2 or ASO-ctl for 48 hours. ASO1 and ASO3 serve as two additional negative controls, due to their incapability of inhibiting AZIN1 editing. FIG. 5C shows foci formation assay of each of three cell lines, after being treated with ASO3.2 or ASO-ctl at the indicated concentrations for 48 hours. Cells were stained with crystal violet. The results in FIGS. 5B and 5C show that ASO3.2 could specifically inhibit cell viability of cancer cells which express edited AZIN1^{S367G}. FIG. 5D left panel: cells were treated with 50 nM of ASO3, ASO3.2 or ASO-ctl for 48 hours, followed by PI staining and cell cycle analysis by flow cytometry. The original FACS data were analysed with BD FACSDiva Software which plots the cell count versus DNA content. FIG. 5D right panel: bar charts showing the percentage of cells at sub-G1, G1, S and G2/M phases from a representative experiment. The results in FIG. 5D show that upon treatment of ASO3.2, KYSE510 and H358 cells demonstrated an obvious attenuation of GUS transition and a dramatic increase in the percentage of sub-G1 phase (apoptotic cells) when compared to cells treated with ASO-ctl or ASO3. FIG. 5E shows results of Western blot analyses of CCND1 and ODC protein expression in KYSE510 cells described in FIG. 5D. GAPDH was used as the loading control. The results in FIG. 5E show that a significant reduction in CCND1 and ODC protein expression was observed cells treated with ASO3.2, thus supporting the GUS arrest induced by ASO3.2 shown in FIG. 5D.

FIG. 6 shows that ASO3.2 specifically inhibits tumour incidence and growth in vivo. FIG. 6A shows cumulative tumor incidence curves of NOD scid gamma (NSG) mice subcutaneously injected with KYSE510 cells pre-treated with 100 nM of ASO3.2 or ASO-ctl for 48 hours, estimated by the Kaplan-Meier method. ASO3.2 or ASO-ctl pre-treated cells were injected into right or left dorsal flank of mice, respectively. The results in FIG. 6A show that tumor incidence rate of the ASO3.2 pre-treated group was markedly lower than that of the ASO-ctl pre-treated group. FIG. 6B shows representative tumors derived from pre-treated KYSE510 cells as described above, 6 weeks after subcutaneous injection (n=6 mice per group), as well as growth curves of tumors derived from each group of pre-treated cells over a period of 6 weeks. Data are presented as the mean±s.d. **P<0.01, ***P<0.001 determined by unpaired, two-tailed Student's t test. The results in FIG. 6B show that during a 6-week observation period, tumors derived from ASO-ctl pre-treated cells grew significantly faster than tumors derived from ASO3.2 pre-treated cells. FIG. 6C shows representative fluorescence microscopy images of KYSE510 cells treated with ASO3.2 loaded into CFSE-labelled RBCEVs. DAPI staining indicates the nuclei. Scale bar, 500 m. The results in FIG. 6C show that the majority of ASO3.2-RBCEVs could enter the cells. FIG. 6D representative tumors derived from KYSE510 cells after receiving intratumoral (i.t.) injection of ASO3.2-RBCEVs or ASO-ctl-RBCEVs every 4 days (n=6 mice per group). For each injection, a total of 1 g ASO was loaded into 50 μg of RBCEVs and resuspended in 20 μL of PBS. Growth curves of each group of tumors over a period of 7 weeks are shown. Data are presented as the mean±s.d. *P<0.05, **P<0.01 determined by unpaired, two-tailed Student's t test. Black arrow indicates each injection. The results in FIG. 6D show that intratumoral injection of ASO3.2-RBCEVs significantly inhibited tumor growth. FIG. 6E shows representative tumors after receiving multiple i.t. injection of naked ASO3.2 or ASO-ctl. The same experimental procedures were conducted as described in FIG. 6D. The results in FIG. 6E show that there was no obvious difference observed in tumor growth between mice treated with naked ASO-ctl and ASO3.2. FIG. 6F shows sequencing chromatograms illustrating editing of AZIN1 transcripts in the indicated PDX lines. Percentage of editing is calculated as area of “G” peak over the total area of “A” and “G” peaks. Black arrow indicates the position of editing site. *, no editing detected. The results in FIG. 6F show that four PDX cells (PDX-1; and PDX-22-T1, T4 and T5 which are from different sectors in PDX-22) have more than 20% of edited AZIN1 transcripts. FIG. 6G shows cell viability of PDX1 (top panel) or PDX22-T3 (bottom panel) measured by CTG assays, after the treatment with the indicated concentrations of ASO 3.2 or ASO-ctl (delivered by lipofectamine) for 48 hours. Data are presented as the mean±s.d. of four replicates from a representative experiment. *P<0.05, **P<0.01, ***P<0.001 determined by unpaired, two-tailed Student's t test. Taken together, the results in FIGS. 6F and 6G show that ASO3.2 treatment can significantly reduce cell viability in an AZIN1 editing-positive cell line, but not in a non-AZIN1 editing cell line.

FIG. 7 shows result of quantitative real-time PCR (QPCR) analysis of ADAR1 expression in the HEK293T cells co-transfected with the pRK7 minigenes as indicated and empty vector (EV) or ADAR1 expression construct (ADAR1). The result indicates successful ADAR1 overexpression in all samples co-transfected with ADAR1 expression construct.

FIG. 8 shows results of quantitative real-time PCR (QPCR) analysis of ADAR1 expression in the HEK293T cells co-transfected with the pRK7 minigenes as indicated and empty vector (EV) or ADAR1 expression construct (ADAR1). The result indicates successful ADAR1 overexpression in all samples co-transfected with ADAR1 expression construct.

FIG. 9A shows predicted dsRNA secondary structure of the 86-nt AZIN1 duplex probe used for REMSA by RNAFold. FIG. 9B shows REMSA data showing the binding of ASP1, DSP1, or DSP2 to a truncated AZIN1 duplex probe. The truncated RNA duplex was at 0.25 μM. ASP1 concentrations from left to right were at 0, 0.005, 0.01, 0.02, 0.05, 0.1, 0.2, 0.4, 0.7, 1, 1.5, and 2 μM, respectively. ASP1 shows not binding up to 2 μM. As for DSP1 and DSP2, the truncated RNA duplex was at 1 μM. The concentrations of DSP1 and DSP2 from left to right were at 0, 0.01, 0.02, 0.05, 0.1, 0.2, 0.4, 1, 2, 4, 10, and 20 μM, respectively. Both DSP1 and DSP2 show binding to the RNA duplex at M concentrations. FIG. 9B also shows the full sequences of the ASOs and PNAs and their positions on the short duplex of the editing region and ECS region on exon 11 and exon 12 of AZIN1. The results in FIG. 9B show that ASP1 was incapable of binding to the shortened AZIN1 RNA duplex, while DSP1 and DSP2 could bind through PNA-dsRNA triplex formation with a modest binding affinity. FIG. 9C shows sequencing chromatograms of in vitro RNA editing analysis of AZIN1 transcripts transcribed from the FE minigene, after the incubation with purified ADAR1 protein and 10 μM (left) and 200 nM (right) of DSP1 or DSP2. Percentage of editing is calculated as area of “G” peak over the total area of “A” and “G” peaks. Arrow indicates the position of editing site. *, no editing detected. The results in FIG. 9C show that DSP1 and DSP2 were able to abolish AZIN1 editing at a concentration of 10 μM, but their editing inhibitory effects were dramatically attenuated at 200 nM.

FIG. 10A shows sequencing chromatograms illustrating editing of AZIN1 transcripts in the indicated HCC, ESCC and NSCLC cell lines. Percentage of editing is calculated as area of “G” peak over the total area of “A” and “G” peaks. Arrow indicates the position of editing site. *, no editing detected. The results in FIG. 10A show that among the HCC, ESCC and NSCLC cell lines screened, AZIN1 editing was only detected in an ESCC line KYSE510 and a NSCLC line H358. FIG. 10B shows sequencing chromatograms illustrating exon 11 skipping of AZIN1 transcripts detected in KYSE510 cells treated with 100 nM of ASO1.1 for 48 hours.

### DEFINITIONS

As used herein, the term “oligonucleotide” refers to an oligomeric compound comprising a plurality of linked nucleotides. As used herein, the term “oligomeric compound” refers to a polymeric structure comprising two or more sub-structures and capable of hybridizing to a region of a nucleic acid molecule. In some examples, oligonucleotides can be introduced in the form of single-stranded, double-stranded, circular, branched or hairpins and can contain structural elements such as internal or terminal bulges or loops. Double-stranded oligonucleotides can be formed by two oligonucleotide strands hybridized together, or a single oligonucleotide strand with sufficient self-complementarity to allow for hybridization and formation of a fully or partially double-stranded compound.

As used herein, the term “nucleoside” means a glycosylamine comprising a nucleobase and a sugar. Nucleosides includes, but are not limited to, natural nucleosides, abasic nucleosides, modified nucleosides, and nucleosides having mimetic bases and/or sugar groups. As used herein, the term “natural nucleoside” or “unmodified nucleoside” means a nucleoside comprising a natural nucleobase and a natural sugar. Natural nucleosides include RNA and DNA nucleosides. As used herein, the term “nucleobase” refers to the base portion of a nucleoside or nucleotide. A nucleobase may comprise any atom or group of atoms capable of hydrogen bonding to a base of another nucleic acid. As used herein, the term “natural nucleobase” refers to a nucleobase that is unmodified from its naturally occurring form in RNA or DNA. Examples of “natural nucleobases” include the purine nucleobases adenine (A) and guanine (G), and the pyrimidine nucleobases thymine (T), cytosine (C) and uracil (U). In addition to the “natural nucleobases”, many modified nucleobases or nucleobase mimetics known to those skilled in the art are amenable with the compounds described herein. The terms “modified nucleobase” and “nucleobase mimetic” can overlap but generally a “modified nucleobase” refers to a nucleobase that is fairly similar in structure to the parent nucleobase, such as for example a 7-deaza purine, a 5-methyl cytosine, or a G-clamp, whereas a “nucleobase mimetic” would include more complicated structures, such as for example a tricyclic phenoxazine nucleobase mimetic.

As used herein, the term “nucleotide” refers to a nucleoside having a phosphate group covalently linked to the sugar. Nucleotides may be modified with any of a variety of substituents.

As used herein, the term “targeting” or “targeted to” refers to the association of a compound to a particular target nucleic acid molecule or a particular region of nucleotides within a target nucleic acid molecule. An antisense compound targets a target nucleic acid if it is sufficiently complementary to the target nucleic acid to allow hybridization under physiological conditions.

The term “RNA editing” as used herein refers to co- or post-transcriptional modification process which introduces changes in RNA sequences encoded by the genome, contributing to RNA mutations. Editing of adenosine to inosine (A-to-I) in double-stranded RNA (dsRNA), catalyzed by adenosine deaminase acting on RNA (ADAR) family of enzymes, is a common type of RNA editing in mammals. In vertebrates, a family of three ADAR proteins, ADAR1, ADAR2 and ADAR3, has been previously characterized. ADAR1 and ADAR2 (ADARs) catalyze all currently known A-to-I editing sites. ADAR3 has no known deaminase activity. Inosine (I) mimics guanosine (G), therefore ADAR proteins introduce a virtual A-to-G substitution in transcripts. Such changes can lead to specific amino acid substitutions, alternative splicing, microRNA-mediated gene silencing, or changes in transcript localization and stability.

The term “AZIN1 gene” as used herein refers to the gene that encodes antizyme inhibitor 1 protein. Antizyme inhibitor 1 belongs to the antizyme inhibitor family, which plays a role in cell growth and proliferation by maintaining polyamine homeostasis within the cell. Antizyme inhibitors are homologs of ornithine decarboxylase (ODC, the key enzyme in polyamine biosynthesis) that have lost the ability to decarboxylase ornithine but retained the ability to bind to antizymes. Antizymes negatively regulate intracellular polyamine levels by binding to ODC and targeting it for degradation, as well as by inhibiting polyamine uptake. Antizyme inhibitors function as positive regulators of polyamine levels by sequestering antizymes and neutralizing their effect. Antizyme inhibitor 1 is ubiquitously expressed and localized in the nucleus and cytoplasm of cells. Overexpression of AZIN1 gene has been associated with increased proliferation, cellular transformation and tumorigenesis. In one specific example, the sequence of AZIN1 gene is SEQ ID NO: 3, encoding the protein of SEQ ID NO: 4.

An “ADAR enzyme” is a double-stranded RNA-specific adenosine deaminase enzyme capable of modifying a polynucleotide at specific nucleic acids (e.g., mRNA). In some examples, an ADAR enzyme performs post-transcriptional modification, or “editing” of an mRNA sequence, for example, by converting an adenosine to inosine. As inosine mimics the activity of a guanosine (e.g., pairing with cytosine), this can effectively result in the formation of a single-nucleotide polymorphism in the transcribed mRNA sequence. In some examples, editing can result in the formation a “cryptic” splice site, recombination motif, or other nucleic acid element.

The term “editing-site complementary sequence” or “ECS” in short as used herein refers to a sequence in the untranslated region (UTR), exon or intron of the gene being edited, which is able to form a double-stranded RNA structure with the sequence covering an adenosine-to-inosine editing site and its surrounding region. In some examples, the ECS is in the intron of the gene being edited. In some examples, the ECS is able to form an imperfect fold-back double-stranded RNA structure with the exon sequence surrounding an adenosine-to-inosine editing side. In some examples, the ECS of AZIN1 for ADAR1 mediated pre-mRNA editing comprises or consists of the 29-nucleotide sequence 5′-AAGAAGACAGCUUUUCCGCUGAAGCUUAA-3′ (SEQ ID NO: 1) located near the 3′ end of exon 12 of AZIN1. The term “core ECS” as used herein in the context of “core ECS of AZIN1 for ADAR1 mediated pre-mRNA editing” refers to a particular part of the ECS that was found by the inventors of the present application to be critical for the ADAR1 mediated pre-mRNA editing of AZIN1 (i.e. the deletion of the core ECS results in the inhibition of ADAR1 mediated pre-mRNA editing of AZIN1). In some examples, the core ECS of AZIN1 for ADAR1 mediated pre-mRNA editing comprises or consists of the 8-nucleotide sequence 5′-GCTTTTCC-3′ located near the 3′ end of exon 12 of AZIN1.

The term “editing region” as used herein refers to a sequence in the gene, for example, in the AZIN1 gene, recognized and/or targeted by ADAR-1 for editing. In some examples, the editing region of AZIN1 for ADAR1 mediated pre-mRNA editing comprises or consists of the sequence 5′-UGAGCUUGAUCAAAUUGUGGAAAGCUGUCUUCUUCCUGAGCU-3′ (SEQ ID NO: 2) located in exon 11 of AZIN1 (with the underlined “A” being the adenosine-to-inosine editing site). In some examples, the sequence 5′-GGAAAGC-3′ is regarded as the “editing site-containing sequence or region”. It is generally understood that in the absence of the adenosine-to-inosine editing site, ADAR1 mediated pre-mRNA editing will not take place.

The term “sugar modification” or “modified sugar” as used interchangeably herein refers to a sugar moiety that is not a ribofuranosyl as found in naturally occurring RNA or a deoxyribofuranosyl as found in naturally occurring DNA. Modified sugar moieties can be used to alter, typically increase, the affinity of the antisense compound for its target and/or increase nuclease resistance. A “modified sugar” includes but is not limited to a substituted sugar, a bicyclic or tricyclic sugar, or a sugar surrogate. As used herein, “substituted sugar moiety” means a furanosyl comprising at least one substituent group that differs from that of a naturally occurring sugar moiety. Substituted sugars include, but are not limited to, furanosyls comprising substituents at the 2′-position, the 3′-position, the 5′-position and/or the 4′-position. As used herein, “2′-substituted sugar” means a furanosyl comprising a substituent at the 2′-position other than H or OH. Unless otherwise indicated, a 2′-substituted sugar is not a bicyclic sugar (i.e., the 2′-substituent of a 2′-substituted sugar moiety does not form a bridge to another atom of the furanosyl ring). Examples of sugar substituents suitable for the 2′-position include, but are not limited to: 2′-O-methyl, 2′-O-methoxyethyl, and 2′-fluoro. In some examples, sugar substituents at the 2′ position is selected from allyl, amino, azido, thio, O-allyl, O—C₁-C₁₀ alkyl, O—C₁-C₁₀ substituted alkyl; O—C₁-C₁₀ alkoxy; O—C₁-C₁₀ substituted alkoxy, OCF₃, O(CH₂)₂SCH₃, O(CH₂)₂—O—N(Rm)(Rn), and O—CH₂—C(═O)—N(Rm)(Rn), where each Rm and Rn is, independently H or substituted or unsubstituted C₁-C₁₀ alkyl.

As used herein, “bicyclic sugar” means a modified sugar comprising a 4 to 7 membered ring (including but not limited to a furanosyl) comprising a bridge connecting two atoms of the 4 to 7 membered ring to form a second ring, resulting in a bicyclic structure. In some examples, the 4 to 7 membered ring is a sugar ring. In some examples, the 4 to 7 membered ring is a furanosyl. In some of such examples, the bridge connects the 2′-carbon and the 4′-carbon of the furanosyl.

As used herein, the term “bicyclic nucleoside” or “BNA” refers to a nucleoside wherein the furanose portion of the nucleoside includes a bridge connecting two atoms on the furanose ring, thereby forming a bicyclic ring system. BNAs include, but are not limited to, α-L-LNA, β-D-LNA, ENA, Oxyamino BNA (2′-O—N(CH₃)—CH₂-4′) and Aminooxy BNA (2′-N(CH₃)—O—CH₂-4′).

Representative structures of BNA's include but are not limited to:

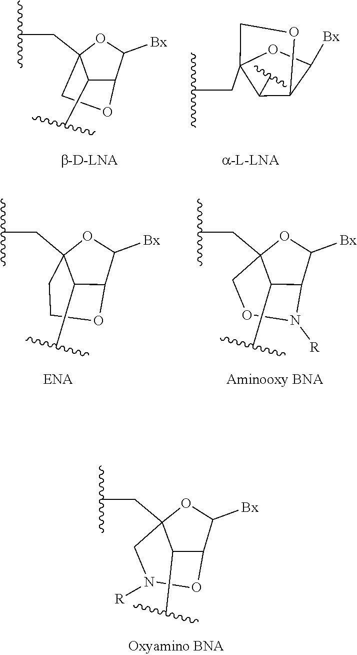

As used herein, the term “4′ to 2′ bicyclic nucleoside” refers to a BNA wherein the bridge connecting two atoms of the furanose ring bridges the 4′ carbon atom and the 2′ carbon atom of the furanose ring, thereby forming a bicyclic ring system.

As used herein, a “locked nucleic acid” or “LNA” refers to a nucleotide modified such that the 2′-hydroxyl group of the ribosyl sugar ring is linked to the 4′ carbon atom of the sugar ring via a methylene groups, thereby forming a 2′-C,4′-C-oxymethylene linkage. LNAs include, but are not limited to, α-L-LNA, and β-D-LNA.

As used herein, the term “sugar surrogate” means a structure that does not comprise a furanosyl and that is capable of replacing the naturally occurring sugar of a nucleoside, such that the resulting nucleoside is capable of (1) incorporation into an oligonucleotide and (2) hybridization to a complementary nucleoside. Such structures include rings comprising a different number of atoms than furanosyl (e.g., 4, 6, or 7-membered rings); replacement of the oxygen of a furanosyl with a non-oxygen atom (e.g., carbon, sulfur, or nitrogen); or both a change in the number of atoms and a replacement of the oxygen. Such structures may also comprise substitutions corresponding to those described for substituted sugar moieties (e.g., 6-membered carbocyclic bicyclic sugar surrogates optionally comprising additional substituents). Sugar surrogates also include more complex sugar replacements (e.g., the non-ring systems of peptide nucleic acid). Sugar surrogates include without limitation morpholino, modified morpholinos, cyclohexenyls and cyclohexitols.

As used herein, the term “Peptide nucleic acid” or “PNA” in short refers to an artificially synthesized polymer similar in structure to DNA or RNA. In some examples, PNAs are resistant to cleavage by RNAi or RNase H, and/or resistant to degradation by nucleases and proteases. PNAs can also have increased stability and longer half-life compared to a comparable oligonucleotide. In some examples, PNAs have a high binding affinity to DNAs and RNAs. In some examples, PNAs comprise a backbone of repeating N-(2-aminoethyl)-glycine units linked by peptide bonds. The various purine and pyrimidine bases are linked to the backbone by a methylene bridge (—CH₂—) and a carbonyl group (—(C═O)—). PNAs are often depicted like peptides, with the N-terminus at the first (left) position and the C-terminus at the last (right) position. In some examples, PNAs have a primary amide at the C-terminus to form a primary amide bond. In some examples, the N-terminus of PNAs comprise a lysine amino acid. In some cases, PNAs have two C-termini or two N-termini. In some examples, the backbone of PNAs does not comprise charged phosphate groups.

As used herein “internucleotide linkage” refers to a covalent linkage between adjacent nucleotides.

As used herein “natural internucleotide linkage” refers to a 3′ to 5′ phosphodiester linkage.

As used herein, the term “modified internucleotide linkage” refers to any linkage between nucleotides other than a naturally occurring internucleotide linkage. Modified internucleotide linkages, compared to natural phosphodiester linkages, can be used to alter, typically increase, nuclease resistance of the antisense compound.

As used herein, the term “antisense compound” refers to an oligomeric compound that is at least partially complementary to a target nucleic acid molecule to which it hybridizes. In some examples, an antisense compound modulates (increases or decreases) expression of a target nucleic acid. Antisense compounds include, but are not limited to, compounds that are oligonucleotides, oligonucleosides, oligonucleotide analogs, oligonucleotide mimetics, and chimeric combinations of these. Consequently, while all antisense compounds are oligomeric compounds, not all oligomeric compounds are antisense compounds.

As used herein, the term “antisense oligonucleotide” refers to an antisense compound that is an oligonucleotide.

As used herein, the term “complementary” refers to the capacity of an oligomeric compound to hybridize to another oligomeric compound or nucleic acid through nucleobase complementarity. In some examples, an antisense compound and its target are complementary to each other when a sufficient number of corresponding positions in each molecule are occupied by nucleobases that can bond with each other to allow stable association between the antisense compound and the target. One skilled in the art recognizes that the inclusion of mismatches is possible without eliminating the ability of the oligomeric compounds to remain in association. Therefore, disclosed herein include antisense compounds that may comprise up to about 20% nucleotides that are mismatched (i.e., are not nucleobase complementary to the corresponding nucleotides of the target). Preferably the antisense compounds contain no more than about 15%, more preferably not more than about 10%, most preferably not more than 5% or no mismatches. The remaining nucleotides are nucleobase complementary or otherwise do not disrupt hybridization (e.g., universal bases). One of ordinary skill in the art would recognize the compounds provided herein are at least 80%, at least 85%, at least 90%, at least 95%, at least 96%, at least 97%, at least 98%, at least 99% or 100% nucleobase complementary to a target nucleic acid.

As used herein, “hybridization” means the pairing of complementary oligomeric compounds (e.g. an antisense compound and its target nucleic acid). While not limited to a particular mechanism, the most common mechanism of pairing involves hydrogen bonding, which may be Watson-Crick, Hoogsteen or reversed Hoogsteen hydrogen bonding, between complementary nucleobases. For example, the natural base adenine is nucleobase complementary to the natural nucleobases thymidine and uracil which pair through the formation of hydrogen bonds. The natural base guanine is nucleobase complementary to the natural bases cytosine and 5-methyl cytosine. Hybridization can occur under varying circumstances.

Two sequences may be complementary and hybridize to each other under moderately stringent, or preferably stringent, conditions. Hybridization to desired sequences under moderately stringent conditions or under stringent conditions can be performed by methods known in the art. Hybridization conditions can also be modified in accordance with known methods depending on the sequence of interest.

As used herein, the term “percent complementary” refers to the number of nucleobases of an oligomeric compound that have nucleobase complementarity with a corresponding nucleobase of another oligomeric compound or nucleic acid, divided by the total length (number of nucleobases) of the oligomeric compound.

The term “percentage identity” as used herein refers to the value determined by the number of matching identical nucleotides or amino acids divided by the sequence length for which the percent identity is being reported. Percent amino acid sequence similarity may be determined by the same calculation as used for determining percent amino acid sequence identity, but may, for example, include conservative amino acid substitutions in addition to identical amino acids in the computation. Oligonucleotide alignment algorithms such as BLAST (GenBank; using default parameters) may be used to calculate sequence percentage identity.

As used herein, the term “pharmaceutically acceptable salts” refers to salts of active compounds that retain the desired biological activity of the active compound and do not impart undesired toxicological effects thereto. Sodium salts of antisense oligonucleotides are useful and are well accepted for therapeutic administration to humans.

As used herein, the term “prodrug” refers to a therapeutic agent that is prepared in an inactive or less active form that is converted to an active form (i.e., drug) within the body or cells thereof by the action of endogenous enzymes, chemicals, and/or conditions. In particular, prodrug versions of the oligonucleotides can be prepared as SATE ((S-acetyl-2-thioethyl) phosphate) derivatives according to the methods disclosed in WO 93/24510 or WO 94/26764. Prodrugs can also include antisense compounds wherein one or both ends comprise nucleobases that are cleaved (e.g., by incorporating phosphodiester backbone linkages at the ends) to produce the active compound.

As used herein, the term “treatment” refers to administering a composition of the invention to effect an alteration or improvement of the disease or condition. Prevention, amelioration, and/or treatment may require administration of multiple doses at regular intervals, or prior to onset of the disease or condition to alter the course of the disease or condition. Moreover, a single agent may be used in a single individual for each prevention, amelioration, and treatment of a condition or disease sequentially, or concurrently.

As used herein, the term “pharmaceutical agent” refers to a substance provides a therapeutic benefit when administered to a subject.

As used herein, the term “therapeutically effective amount” refers to an amount of a pharmaceutical agent that provides a therapeutic benefit to an animal.

As used herein, “administering” means providing a pharmaceutical agent to an animal, and includes, but is not limited to administering by a medical professional and self-administering.

As used herein, the term “pharmaceutical composition” refers to a mixture of substances suitable for administering to an individual. For example, a pharmaceutical composition may comprise an oligonucleotide and a sterile aqueous solution.

As used herein, the term “animal” refers to a human or non-human animal, including, but not limited to, mice, rats, rabbits, dogs, cats, pigs, and non-human primates, including, but not limited to, monkeys and chimpanzees.

### DETAILED DESCRIPTION OF THE PRESENT INVENTION

The inventors of the present application have uncovered that the 3′end sequence of exon 12 is the editing-site complementary sequence (ECS) of AZIN1 that forms double stranded RNA (dsRNA) with the sequence being edited at exon 11 of AZIN1. It has been surprisingly found that compounds, in particular oligonucleotides, which target this ECS, can inhibit ADAR1 mediated pre-mRNA editing of AZIN1. The inhibition of pre-mRNA editing of AZIN1 can effectively reduce viability of AZIN1 pre-mRNA editing associated cancer cells in vitro, and inhibit the occurrence and growth of tumors/cancers that are associated with AZIN1 pre-mRNA editing in vivo. Thus, compounds which target the ECS identified by the inventors of the present application serve as promising therapeutic candidates for tumors/cancers that are associated with AZIN1 pre-mRNA editing.

Thus, in one aspect, there is provided an oligonucleotide targeting the core editing-site complementary sequence (ECS) of AZIN1 gene, wherein the core ECS of AZIN1 gene comprises the sequence 5′-GCTTTTCC-3′, and wherein the oligonucleotide comprises one or more nucleotides with sugar modification and one or more modified internucleotide linkages. The oligonucleotide can inhibit ADAR1 mediated pre-mRNA editing of AZIN1.

As shown in the working examples below, in some examples, AZIN1 pre-mRNA comprises an editing region (for example 5′-UGAGCUUGAUCAAAUUGUGGAAAGCUGUCUUCUUCCUGAGCU-3′ (SEQ ID NO: 2), with the underlined “A” being the adenosine-to-inosine editing site), which is recognized and/or targeted by ADAR-1 for editing. In these examples, ADAR-1 edits sequence 5′-GGAAAGC-3′ to 5′-GGAAIGC-3′, resulting in a mutation in the translated AZIN1 protein.

In some examples, the antisense oligonucleotides as disclosed herein prevent recognition and/or binding of ADAR-1, thereby inhibiting or blocking the activity of ADAR-1. This can be achieved by, for example, preventing formation of dsRNA structure within the AZIN1 pre-mRNA strand, or preventing recognition of the dsRNA structure by ADAR-1.

An oligonucleotide can comprise ribonucleic acids (RNAs) or deoxyribonucleic acids (DNAs). In one specific example, the oligonucleotide is an RNA oligonucleotide. In some examples, the oligonucleotide is not a peptide nucleic acid (PNA).

In some examples, the oligonucleotides as disclosed herein are of at least about 8 nucleotides in length, for example, but not limited to about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more nucleotides in length. Further, the length of the oligonucleotides may be defined by a range of any two values as provided above or any two values in between. In some specific examples, the oligonucleotides are of about 20 to 30 nucleotides in length. In one specific example, the oligonucleotide is at least about 20 nucleotides in length. In one specific example, the oligonucleotide is of about 20 nucleotides in length.

In some examples, at least about 30%, 35%, 40%, 45%, 50%, 55%, 60%, 65%, 70%, 75%, 80%, 85%, 90%, 95%, 98% or 100% of the nucleotides in the oligonucleotide are modified with sugar modification. Further, the percentage of nucleotides modified with sugar modification may be defined by a range of any two values as provided above or any two values in between. In one specific example, at least about 50% of the nucleotides in the oligonucleotide are modified with sugar modification. In another specific example, at least about 70% of the nucleotides in the oligonucleotide are modified with sugar modification. In yet another specific example, all the nucleotides in the oligonucleotide are modified with sugar modification.

In some examples, nucleotides modified with sugar modification are located at or near the 5′ end of the oligonucleotide. In some other examples, nucleotides modified with sugar modification are located at or near the 3′ end of the oligonucleotide. In some examples, at least 1, 2, 3, 4, 5, 6, 7, 8, 9 or 10 nucleotides at or near the 5′ end of the oligonucleotide are modified with sugar modification. In some other examples, at least 1, 2, 3, 4, 5, 6, 7, 8, 9 or 10 nucleotides at or near the 3′ end of the oligonucleotide are modified with sugar modification.

In some examples, the nucleotide with sugar modification is 2′-O-methyl modified nucleotide, 2′-O-methoxyethyl modified nucleotide, 2′-fluoro modified nucleotide, 2′,4′-bridged nucleic acid modified nucleotide, locked nucleic acid (LNA) modified nucleotide, or morpholine ring modified nucleotide. In one specific example, the nucleotide with sugar modification is 2′-O-methyl modified nucleotide. In one specific example, all the nucleotides in the oligonucleotide are modified with 2′-O-methyl sugar modification.

When there are more than one nucleotide with sugar modification in the oligonucleotide, these nucleotides can be modified with the same sugar modification, or with different sugar modifications.

In some examples, the oligonucleotides as disclosed herein are antisense oligonucleotides. In some examples, the antisense oligonucleotides are non-degrading antisense oligonucleotides, i.e. the antisense oligonucleotides do not enable target degradation by RNase H or RNA interference (RNAi) mechanisms. In some examples, non-degrading antisense oligonucleotides bind to their target RNA and sterically deny other molecules access for base pairing to the RNA. In some specific examples, such steric blocking antisense oligonucleotides are fully modified at the 2′ sugar position so that RNase H is unable to degrade the target RNA.

In some examples, the oligonucleotides as disclosed herein comprise one or more modified internucleotide linkages. Examples of modified internucleotide linkages include but are not limited to, phosphorus containing internucleoside linkages such as phosphotriesters, methylphosphonates, phosphoramidate, phosphorodiamidate, and phosphorothioates. In some specific examples, the oligonucleotides as disclosed herein comprise one or more phosphorothioate, phosphoramidate, or phosphorodiamidate linkages.

In some examples, at most about 2%, 5%, 10%, 15%, 20%, 25%, 30%, 35%, 40%, 45% or 50% of the internucleotide linkages in the oligonucleotide are modified internucleotide linkages. In some other examples, at least about 2%, 5%, 10%, 15%, 20%, 25%, 30%, 35%, 40%, 45%, 50%, 55%, 60%, 65%, 70%, 75%, 80%, 85%, 90%, 95%, 98% or 100% of the internucleotide linkages in the oligonucleotide are modified internucleotide linkages. Further, the percentage of modified internucleotide linkages in the oligonucleotide may be defined by a range of any two values as provided above or any two values in between. In one specific example, at least about 10% of the internucleotide linkages in the oligonucleotide are modified internucleotide linkages. In one specific example, about 25% of the internucleotide linkages in the oligonucleotide are modified internucleotide linkages. In another specific example, all the internucleotide linkages in the oligonucleotide are modified internucleotide linkages. In yet another specific example, at least about 10% of the internucleotide linkages in the oligonucleotide are phosphorothioate linkages. In yet another specific example, about 25% of the internucleotide linkages in the oligonucleotide are phosphorothioate linkages. In yet another specific example, all the internucleotide linkages in the oligonucleotide are phosphorothioate linkages.

In some examples, modified internucleotide linkages are located at or near the 5′ end of the oligonucleotide. In some other examples, modified internucleotide linkages are located at or near the 3′ end of the oligonucleotide. In some examples, at least 1, 2, 3, 4, 5, 6, 7, 8, 9 or 10 internucleotide linkages at or near the 5′ end of the oligonucleotide are modified internucleotide linkages. In some other examples, at least 1, 2, 3, 4, 5, 6, 7, 8, 9 or 10 internucleotide linkages at or near the 3′ end of the oligonucleotide are modified internucleotide linkages.

In one specific example, when the nucleotide with sugar modification is morpholine ring modified nucleotide, and the modified internucleotide linkage linking the morpholine ring modified nucleotide to the adjacent nucleotide is phosphorodiamidate internucleotide linkage, phosphorodiamidate morpholino oligomers (PMOs) are formed.

In some examples, the oligonucleotides comprise or consist of an antisense sequence that is complementary to the core ECS of AZIN1 gene, such that the oligonucleotides can effectively target the ECS of AZIN1 gene. Since the core ECS of AZIN1 gene comprises the sequence 5′-GCTTTTCC-3′, the antisense sequence that is fully complementary to the core ECS of AZIN1 gene is 5′-GGAAAAGC-3′. One skilled in the art would recognize that the inclusion of mismatches is possible without eliminating the complementary activity of the antisense sequence. Therefore, in some examples, the antisense sequence that is complementary to the core ECS of AZIN1 gene may contain up to 1, 2, or 3 nucleotides that do not form base pairing with the core ECS of AZIN1 gene.

In some examples, the antisense sequence that is complementary to the core ECS of AZIN1 gene is located at or near the 3′ end of oligonucleotide targeting the ECS of AZIN1 gene. For example, the 3′ end of the antisense sequence that is complementary to the core ECS of AZIN1 gene can be at most 0, 1, 2, 3, 4, 5, 6, 7, 8, 9 or 10 nucleotides away from the 3′ end of the oligonucleotide.

The antisense sequence that is complementary to the core ECS of AZIN1 gene is critical for the oligonucleotides comprising the antisense sequence to effectively target the ECS of AZIN1 gene. Thus, in some examples, at least some of the nucleotides in the antisense sequence complementary to the core ECS of AZIN1 gene are modified with sugar modification. This may increase the affinity of the antisense sequence to ECS of AZIN1 gene, or increase nuclease resistance of the antisense sequence. In some examples, at least 5, 6, 7 or 8 of the nucleotides in the antisense sequence complementary to the core ECS of AZIN1 gene are modified with sugar modification. In some specific examples, at least 5, 6, 7 or 8 of the nucleotides in the antisense sequence complementary to the core ECS of AZIN1 gene are 2′-O-methyl modified nucleotides, 2′-O-methoxyethyl modified nucleotides, 2′-fluoro modified nucleotides, 2′,4′-bridged nucleic acid modified nucleotides, locked nucleic acid (LNA) modified nucleotides, or morpholine ring modified nucleotides, or combinations thereof. In one specific example, at least 5, 6, 7 or 8 of the nucleotides in the antisense sequence complementary to the core ECS of AZIN1 gene are 2′-O-methyl modified nucleotides. In another specific example, all the nucleotides in the antisense sequence complementary to the core ECS of AZIN1 gene are 2′-O-methyl modified nucleotides.

In some other examples, at least some of the internucleotide linkages in the antisense sequence complementary to the core ECS of AZIN1 gene are modified internucleotide linkages. This may increase nuclease resistance of the antisense sequence. In some examples, at least 3, 4, 5, 6 or 7 of the internucleotide linkages in the antisense sequence complementary to the core ECS of AZIN1 gene are modified internucleotide linkages. In some specific examples, at least 3, 4, 5, 6 or 7 of the internucleotide linkages in the antisense sequence complementary to the core ECS of AZIN1 gene are phosphorothioate, phosphoramidate, or phosphorodiamidate linkages, or combinations thereof. In one specific example, at least 3, 4, 5, 6 or 7 of the internucleotide linkages in the antisense sequence complementary to the core ECS of AZIN1 gene are phosphorothioate linkages. In another specific example, at least 5 of the internucleotide linkages in the antisense sequence complementary to the core ECS of AZIN1 gene are phosphorothioate linkages. In yet another specific example, all the internucleotide linkages in the antisense sequence complementary to the core ECS of AZIN1 gene are phosphorothioate linkages.

In some examples, none of the internucleotide linkages in the antisense sequence complementary to the core ECS of AZIN1 gene are modified internucleotide linkages, i.e. all the internucleotide linkages in the antisense sequence complementary to the core ECS of AZIN1 gene are the natural 3′ to 5′ phosphodiester linkage. In such examples, other part(s) of the oligonucleotide (i.e. the part(s) that are not complementary to the core ECS of AZIN1 gene) can contain modified internucleotide linkages, so as to increase the nuclease resistance of the oligonucleotide.

In some examples, the oligonucleotide is fully modified with sugar modification and internucleotide linkage modification, i.e. each nucleotide in the oligonucleotide is modified with sugar modification and is connected to an adjacent nucleotide via a modified internucleotide linkage. In some specific examples, each nucleotide in the oligonucleotide is modified with 2′-O-methyl sugar modification and is connected to an adjacent nucleotide via a phosphorothioate linkage.

In some examples, the oligonucleotides as disclosed herein are of at least 70%, 75%, 80%, 85%, 90%, 95%, 98%, or 99% identity to the sequence 5′-UUAAGCUUCAGCGGAAAAGC-3′ (SEQ ID No: 5). In some examples, the oligonucleotides comprise or consist of the sequence 5′-UUAAGCUUCAGCGGAAAAGC-3′ (SEQ ID No: 5). The sequence 5′-UUAAGCUUCAGCGGAAAAGC-3′ (SEQ ID No: 5) contains one or more nucleotides with sugar modification as described in the present application, and optionally one or more modified internucleotide linkages as described in the present application.

In some examples, the oligonucleotides are of at least 70%, 75%, 80%, 85%, 90%, 95%, 98%, or 99% identity to one or more of the following sequences: 5′-mUmUmAmAmGmCmUmUmCmAmGmCmGmGmAmAmAmAmGmC-3′ (SEQ ID No: 6), 5′-mU*mU*mA*mA*mG*mC*mU*mU*mC*mA*mG*mC*mG*mG*mA*mA*mA*mA*mG*mC-3′ (SEQ ID No: 7), 5′-mU*mU*mA*mA*mG*mCmUmUmCmAmGmCmGmGmAmAmAmAmGmC-3′ (SEQ ID No: 8), and 5′-mUmUmAmAmGmCmUmUmCmAmGmCmGmG mA*mA**mA*mG*mC-3′ (SEQ ID No: 9), wherein m represents 2′-O-Me sugar modification, and * represents phosphorothioate linkage. In some examples, the oligonucleotides comprise or consist of one of the following sequences: 5′-mUmUmAmAmGmCmUmUmCmAmGmCmGmGmAmAmAmAmGmC-3′ (SEQ ID No: 6), 5′-mU*mU*mA*mA*mG*mC*mU*mU*mC*mA*mG*mC*mG*mG*mA*mA*mA*mA*mG*mC-3′ (SEQ ID No: 7), 5′-mU*mU*mA*mA*mG*mCmUmUmCmAmGmCmGmGmAmAmAmAmGmC-3′ (SEQ ID No: 8), and 5′-mUmUmAmAmGmCmUmUmCmAmGmCmGmGmA*mA*mA*mA*mG*mC-3′ (SEQ ID No: 9), wherein m represents 2′-O-Me sugar modification, and * represents phosphorothioate linkage.

In some examples, the oligonucleotides as disclosed herein can be labeled by an appropriate moiety known in the art, for example, but not limited to one or more fluorophores, radioactive groups, chemical substituents, enzymes, antibodies or the like, to facilitate identification in hybridization assays and other assays or tests.

The oligonucleotides provided herein can be utilized in pharmaceutical compositions, by for example, adding an effective amount of the oligonucleotide to a suitable pharmaceutically acceptable diluent or carrier. Thus, in one aspect, there is provided a pharmaceutical composition comprising the oligonucleotide as disclosed herein.

Acceptable carriers and diluents are well known to those skilled in the art. Selection of a diluent or carrier is based on a number of factors, including, but not limited to, the solubility of the oligonucleotide and the route of administration. Such considerations are well understood by those skilled in the art.

The oligonucleotides provided herein comprise any pharmaceutically acceptable salts, esters, or salts of such esters, or any other functional chemical equivalent which, upon administration to an animal including a human, is capable of providing (directly or indirectly) the biologically active metabolite or residue thereof. Accordingly, for example, the disclosure also provides prodrugs and pharmaceutically acceptable salts of the oligonucleotides, pharmaceutically acceptable salts of such prodrugs, and other bioequivalents.

The oligonucleotides disclosed herein may also be admixed, encapsulated, conjugated or otherwise associated with other molecules, molecule structures or mixtures of compounds.

The pharmaceutical compositions may be administered in a number of ways depending upon whether local or systemic treatment is desired and upon the area to be treated.

The pharmaceutical formulations described herein, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers, finely divided solid carriers, or both, and then, if necessary, shaping the product (e.g., into a specific particle size for delivery).

A “pharmaceutical carrier” can be a pharmaceutically acceptable solvent, suspending agent or any other pharmacologically inert vehicle for delivering one or more nucleic acids to an animal and are known in the art. The carriers can be liquid or solid and is selected, with the planned manner of administration in mind, so as to provide for the desired bulk, consistency, etc., when combined with a nucleic acid and the other components of a given pharmaceutical composition. Liquid carriers can be aqueous carriers, non-aqueous carriers or both, and include, but are not limited to, aqueous suspensions, oil emulsions, water-in-oil emulsions, water-in-oil-in-water emulsions, site-specific emulsions, long-residence emulsions, sticky-emulsions, micro-emulsions and nano-emulsions. Solid carriers can be biological carriers, chemical carriers or both, and include, but are not limited to, viral vector systems, particles, microparticles, nanoparticles, microspheres, nanospheres, minipumps, bacterial cell wall extracts and biodegradable or non-biodegradable natural or synthetic polymers that allow for sustained release of the oligonucleotide compositions.

Preferred aqueous carriers include, but are not limited to, water, saline and pharmaceutically acceptable buffers. Preferred non-aqueous carriers include, but are not limited to, a mineral oil or a neutral oil including, but not limited to, a diglyceride, a triglyceride, a phospholipid, a lipid, an oil and mixtures thereof, wherein the oil contains an appropriate mix of polyunsaturated and saturated fatty acids. Examples include, but are not limited to, squalene, soybean oil, canola oil, palm oil, olive oil and myglyol, wherein the fatty acids can be saturated or unsaturated. Optionally, excipients may be included regardless of the pharmaceutically acceptable carrier. These excipients include, but are not limited to, anti-oxidants, buffers, and bacteriostats, and may include suspending agents and thickening agents.

Embodiments in which the compositions of the invention are combined with, for example, one or more pharmaceutically acceptable carriers or excipients may be prepared by conventional pharmaceutical techniques. Such techniques include the step of bringing into association the compositions containing the active ingredient and the pharmaceutical carrier(s) or excipient(s). In general, the formulations are prepared by uniformly and intimately bringing into association the active ingredient with liquid carriers.

The composition, shape, and type of dosage forms of the pharmaceutical composition as disclosed herein will typically vary depending on the intended use. For example, a dosage form used in the acute treatment of a disease or a related disease may contain larger amounts of one or more of the active compound it comprises than a dosage form used in the chronic treatment of the same disease. Similarly, a parenteral dosage form may contain smaller amounts of one or more of the active compound it comprises than an oral dosage form used to treat the same disease or disorder. These and other ways in which specific dosage forms encompassed by this invention will vary from one another will be readily apparent to those skilled in the art. Examples of dosage forms include, but are not limited to: tablets; caplets; capsules, such as soft elastic gelatine capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms particularly suitable for parenteral administration to a patient; and sterile solids (e.g., crystalline or amorphous solids) that can be reconstituted to provide liquid dosage forms suitable for parenteral administration to a patient. Thus, in one example, the pharmaceutical composition as disclosed herein is provided in a form selected from, but not limited to, tablets, caplets, capsules, hard capsules, soft capsules, soft elastic gelatine capsules, hard gelatine capsules, cachets, troches, lozenges, dispersions, suppositories, ointments, cataplasms, poultices, pastes, powders, dressings, creams, plasters, solutions, injectable solutions, patches, aerosols, nasal sprays, inhalers, gels, suspensions, aqueous liquid suspensions, non-aqueous liquid suspensions, oil-in-water emulsions, a water-in-oil liquid emulsions, solutions, sterile solids, crystalline solids, amorphous solids, solids for reconstitution or combinations thereof.

In one aspect, there is provided a method of inhibiting AZIN1 pre-mRNA editing in a cell, the method comprising contacting the cell with the oligonucleotides as disclosed herein, or the pharmaceutical compositions as disclosed herein. Such methods can be in vivo, ex vivo or in vitro. Particularly, the AZIN1 pre-mRNA editing inhibited by the oligonucleotides or pharmaceutical compositions as disclosed herein is mediated by adenosine deaminase acting on RNA-1 (ADAR-1).

Methods whereby bodily fluids, organs or tissues are contacted with an effective amount of one or more of the oligonucleotides or pharmaceutical compositions provided herein are also contemplated. Bodily fluids, organs or tissues can be contacted with one or more of the oligonucleotides, resulting in modulation of AZIN1 pre-mRNA editing in the cells of bodily fluids, organs or tissues. An effective amount can be determined by monitoring the modulatory effect of the oligonucleotides or pharmaceutical compositions on AZIN1 pre-mRNA editing by methods routine to a person skilled in the art.

Pre-mRNA editing of AZIN1 gene has been associated with increased proliferation, cellular transformation and tumorigenesis. Thus, the oligonucleotides as disclosed herein, which are capable of inhibiting ADAR-1 mediated AZIN1 pre-mRNA editing, can be effective in treating cancers that are associated with ADAR-1 mediated AZIN1 pre-mRNA editing. Therefore, in one aspect, there is provided a method of treating cancer in a subject in need thereof, comprising administering to the subject a therapeutically effective amount of the oligonucleotide as disclosed herein, or the pharmaceutical composition as disclosed herein, wherein the cancer is associated with AZIN1 pre-mRNA editing.

Protein-recoding type of RNA editing can contribute to tumorigenesis through enhancing the activity of oncogenes or reducing the activity of tumor suppressors. In some examples, AZIN1 pre-mRNA can be edited by ADAR1 protein, resulting in a serine (S) to glycine (G) substitution at residue 367. In some examples, AZIN1^{S367G} is more stable than the wild-type AZIN1, and have a stronger affinity to antizyme. Antizyme regulates growth by binding and degrading proteins associated with cell growth and proliferation, such as ornithine decarboxylase (ODC) and cyclin D1 (CCND1). AZIN1^{S367G} can inhibit antizyme-mediated degradation of ODC and CCND1 by competing with wild-type AZIN1 for binding to antizyme, thereby facilitating entry into cell cycle and possessing stronger tumorigenic capabilities than the wild-type AZIN1.

Examples of cancers associated with AZIN1 pre-mRNA editing, in particular ADAR-1 mediated AZIN1 pre-mRNA editing, include but are not limited to, liver cancer, esophageal cancer, lung cancer and colorectal cancer. Specific types of cancers include but are not limited to, hepatocellular carcinoma (HCC), esophageal squamous cell carcinoma (ESCC), non-small cell lung cancer (NSCLC), and colorectal cancer (CRC). In some examples, elevated level(s) of AZIN1 RNA editing is a prognostic factor for overall survival and disease-free survival and an independent risk factor for lymph node and distant metastasis.

In some examples, the methods of treating cancer as described herein comprise administration of plural therapeutic agents. In some examples, any oligonucleotides or pharmaceutical compositions as described herein is a first therapeutic agent, and the methods further comprises administering a second therapeutic agent. The second therapeutic agent can be administered before, concurrent or subsequent to the first therapeutic agent. In some examples, the second therapeutic is an RNA-based therapeutic or a small molecule drug.

It will be understood that a small molecule drug may refer to a drug known in the art for targeting cancer. Examples of appropriate drugs include: sorafenib, gefitinib, osimertinib, crizotinib, pemetrexed (Alimta), paclitaxel, carboplatin, gemcitabine, capecitabine, eribulin, 5-FU (5-fluorouracil) and others. Some drugs may be used in combination with oligonucleotides described herein to treat specific diseases or conditions. For example, when treating non-small cell lung cancer (NSCLC), oligonucleotides described herein may be combined with gefitinib, osimertinib (for EGFR mutants), crizotinib (for ALK mutants) or combinations thereof. In some examples, oligonucleotides as disclosed herein can be combined with a chemo drug such as pemetrexed (Alimta), which is used when tumors do not respond to targeted drugs. In cases where breast cancer is targeted, oligonucleotides described herein may be combined with paclitaxel, carboplatin, gemcitabine, capecitabine, eribulin or combinations thereof. In cases where colon cancer is targeted, oligonucleotides described herein may be combined with 5-FU (5-Flurouracil) or Capecitabine.

Provided herein also include use of the oligonucleotide as disclosed herein, or the pharmaceutical composition as disclosed herein, in the manufacture of a medicament for treating cancer, wherein the cancer is associated with AZIN1 pre-mRNA editing. Provided herein also include the oligonucleotide as disclosed herein, or the pharmaceutical composition as disclosed herein, for use in therapy, in particular for use in the treatment of cancer, wherein the cancer is associated with AZIN1 pre-mRNA editing.

In some examples, oligonucleotides as disclosed herein suppress the growth, cell viability, and/or proliferation of cells, in particular cancer cells, by reducing GUS cell cycle transition. Cancer cells can be understood as any cancer cells described herein, or any cancer cells in which mutated antizyme inhibitor is produced. In some examples, reducing GUS cell cycle transition comprises reducing the amount of mutated antizyme inhibitor that is translated from edited AZIN1 RNA transcripts. Blocking dsRNA formation and/or aberrant editing of AZIN1 RNA transcripts by ADAR-1 results in the reduction of mutated antizyme inhibitor that is produced.

In some examples, in order to determine if the cancer in a patient is associated with AZIN1 pre-mRNA editing and thus should be treated with the oligonucleotides or pharmaceutical compositions as disclosed herein, a sample is obtained from the patient, in order to measure the level of edited AZIN1 pre-mRNA. Thus, in some examples, the method of treatment as disclosed herein further comprises measuring the level of edited AZIN1 pre-mRNA in a sample obtained from the subject, prior to administering a therapeutically effective amount of the oligonucleotide of as disclosed herein, or the pharmaceutical composition as disclosed herein. In some examples, measuring the level of edited AZIN1 pre-mRNA comprises isolating and sequencing of RNA transcripts of AZIN1.

In some examples, in order for the cancer in a patient to be considered as being associated with AZIN1 pre-mRNA editing, the level of edited AZIN1 pre-mRNA in the sample obtained from the patient is at least 15%, 20%, 25%, 30%, 35%, 40%, 45% or 50% higher as compared to the level of edited AZIN1 in a sample obtained from a healthy subject. In the Examples provided, the level of edited AZIN1 pre-mRNA is calculated as area of “G” (indicating editing by ADAR-1) peak over the total area of “A” and “G” peaks.

In some examples, level of edited AZIN1 pre-mRNA is also determined during treatment to indicate efficacy of the treatment, and/or checked after treatment to determine if the treatment was effective.

The term “sample” used herein refers to a biological sample, or a sample that comprises at least some biological materials such as cells, DNAs or RNAs. Examples of biological samples include but are not limited to, solid tissue samples, such as bone marrow, and liquid samples, such as whole blood, blood serum, blood plasma, cerebrospinal fluid, central spinal fluid, lymph fluid, cystic fluid, sputum, stool, pleural effusion mucus, pleural fluid, ascitic fluid, amniotic fluid, peritoneal fluid, saliva, bronchial washes and urine. In some examples, the biological sample is a blood sample. In some other examples, the biological sample is a tumor sample obtained from tumor biopsies or surgically removed tumors.

The biological samples of this disclosure may be obtained from any organism, including mammals such as humans, primates (e.g., monkeys, chimpanzees, orangutans, and gorillas), cats, dogs, rabbits, farm animals (e.g., cows, horses, goats, sheep, pigs), and rodents (e.g., mice, rats, hamsters, and guinea pigs).

It should be understood that following any method as described herein related to testing, identifying or screening, such methods may further comprise additional testing or screening for one or more additional genetic mutations, blood tests, blood enzyme tests, counseling, providing support resources or administering an additional pharmaceutical agent based on the results of such tests and/or screens. Similarly, it is further contemplated that such methods may be preceded by one or more steps, for example but not limited to selecting a subject that has cancer or thought to be at risk of having cancer, or selecting a subject that is pre-cancerous or suspected of being at risk for being pre-cancerous.

Oligonucleotides can be delivered by “naked delivery” which is understood as administration directly to the body and taken up into cells by receptors. Oligonucleotides can also be conjugated to ligands, such as cell-penetrating peptides, neamine, N-acetylgalactosamine (GalNAc). Oligonucleotides can also delivered by a suitable carrier, such as nanoparticles. Transfection, lipofection or electroporation of the oligonucleotide into a cell can also be used.

Methods of administration of oligonucleotides or pharmaceutical compositions as disclosed herein include, but are not limited to the following: oral (e.g. buccal or sublingual), anal, rectal, as a suppository, intracolonic, topical, parenteral, nasal, aerosol, inhalation, intrathecal, intraperitoneal, intravenous, intra-arterial, transdermal, intradermal, subdermal, subcutaneous, intramuscular, intralymphatic, intrauterine, intravesicular, vaginal, visceral, into a body cavity, surgical administration at the location of the inflamed tissue such as adipose tissue, into the lumen or parenchyma of an organ, into bone marrow and into any mucosal surface of the gastrointestinal, reproductive, urinary and genitourinary system. It is to be understood that the choice of route of administration will be selected by one of ordinary skill in the art of treatment such that inhibition or reduction in RNA editing levels is achieved.

It is noted that, as used herein, the terms “organism”, “individual”, “subject”, or “patient” are used as synonyms and interchangeably.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation, or metabolites thereof, in the body of the patient. The administering physician can easily determine optimum dosages, dosing methodologies and repetition rates. Optimum dosages may vary depending on the relative potency of the composition, and can generally be estimated based on arithmetic means, for example based on EC₅₀ values found to be effective in in vitro and in vivo animal models, or based on the examples described herein. In general, dosage of the pharmaceutical composition according to the present disclosure is from about 0.01 μg to 100 g/kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the composition is administered in maintenance doses, ranging from 0.01 μg to 100 g/kg of body weight, once or more daily, to once every 2 years.

As mentioned above, a person skilled in the art would be able to ascertain, based on, for example, disease severity, the required dosage amount and dosage regime required to attain the desired clinical effect. The following is used as an illustrative example of an intravenous injection, which may be amended as required for other modes of administration. In one example, the method, as disclosed herein, is to be administered to a subject as at least one injection. In one example, more than a single injection may be administered to a patient at any given time. In yet another example, the method as disclosed herein may require that a single injection be administered to the patient more than once within a specified treatment timeframe or regime. In yet another example, the method as disclosed herein may require that more than two or more injections be administered to the patient more than once within a specified treatment timeframe or regime. This means, at according to clinical requirements, the subject may be given an initial treatment in the form of an injection, whereby further treatment may follow at interval of, for example, 3 day, 7 days, weekly, 2 weeks, fortnightly, 1 month, monthly, quarterly, biannually, annually or longer, depending on the treatment designed for the subject. If required, the method disclosed herein may also be used as in combination therapy with other drugs or pharmaceutical compositions.

Also described herein are methods of identifying RNA therapeutic agents which modulate pre-mRNA editing. Methods described herein to target AZIN1 pre-mRNA editing may not be limited to AZIN1, but may be used to target genes other than AZIN1. Such methods may comprise determining the editing region and ECS of a desired RNA transcript, determining the dsRNA structure of the desired transcript, and designing RNA therapeutic agents to target and disrupt the assembly of the dsRNA or binding to the dsRNA structure. Antisense oligonucleotides may be designed to disrupt pre-mRNA editing by targeting the dsRNA structure, for example by targeting the editing region or the ECS. Such antisense oligonucleotides may comprise any of the chemical modifications described herein. Any of the steps described may be achieved using the methods described herein. For example, a minigene assay as described herein may be used to determine dsRNA structures of desired target sequences. A minigene is a minimal gene fragment that includes an exon and the control regions necessary for the gene to express itself in the same way as a wild type gene fragment. This is a minigene in its most basic sense. More complex minigenes can be constructed containing multiple exons and intron(s). Minigenes provide a valuable tool for researchers evaluating splicing patterns both in vivo and in vitro biochemically assessed experiments. Specifically, minigenes are used as splice reporter vectors (also called exon-trapping vectors) and act as a probe to determine which factors are important in splicing outcomes. They can be constructed to test the way both cis-regulatory elements (RNA effects) and trans-regulatory elements (associated proteins/splicing factors) affect gene expression.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

It will be understood by those skilled in the art that a wide variety of methods and techniques known in the art may be used in carrying out certain embodiments of the present invention. By way of example, detection of mutations in any oncogenes or tumor-suppressors and other polymorphisms or mutations described herein may be accomplished using a variety of approaches and techniques well-known in the field.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### EXAMPLES

The following examples illustrate methods by which aspects of the invention may be practiced or materials suitable for practice of certain embodiments of the invention may be prepared.

Materials and Methods

Cell Lines

All cell lines were maintained in Roswell Park Memorial Institute (RPMI-1640) media (Biowest) supplemented with 10% FBS (Biowest). All cell lines used in this study were regularly authenticated by morphological observation and tested for mycoplasma contamination. Patient-derived xenografts (PDX) lines were cultured in DMEM/F12 (Biowest) and supplemented with 1:50 B27 supplement without vitamin A (ThermoFisher), 1:100 Insulin-Transferrin-Selenium supplement (Gibco), 1.25 mM N-acetyl-L-cysteine (Sigma-Aldrich), 10 mM nicotinamide (Sigma-Aldrich), 10 nM recombinant human (Leu15)-gastrin I (Sigma-Aldrich), 25 ng/mL recombinant human HGF (Abcam), 50 ng/mL recombinant human EGF (Abcam), 50 ng/mL recombinant human bFGF (Abcam), 5 μg/mL Heparin (Sigma-Aldrich), 10-ng/mL recombinant human FGF-10 (Abcam). All cancer cell lines and PDX lines were incubated at 37° C. in a humidified incubator containing 5% CO₂.

Normal Human Hepatocytes

Human hepatocytes are prepared from mice (Mus musculus) following humanization of the liver with fresh or frozen human liver/hepatocytes derived from cadavers. HepaCur™ Human Hepatocytes are isolated from the perfused livers of humanized FRG® KO mice. The freshly isolated hepatocytes are guaranteed to be ≥95% human and have a viability of ≥70%. Isolated hepatocytes were cultured in HypoThermosol® FRS (BioLife Solutions, Cat #101373) which is an optimized hypothermic preservation media. Prior to plating, HypoThermosol® FRS was changed with HMM (HepaCur™ Maintenance Medium, Catalog #HMM500), according to manufacturer's protocol. Subsequently, 1.0×10⁴ cells were plated into each well of a 96-well plate and treated with ASOs on the same day. The cells were incubated at 37° C. in a humidified incubator containing 5% CO₂.

RNA Extraction, cDNA Synthesis, Quantitative PCR (qPCR) and Sanger Sequencing

Total RNA was extracted using RNeasy Mini Kit (Qiagen), according to the manufacturer's protocol. cDNA synthesis was conducted using the Advantage Reverse Transcription Kit (Clontech Laboratories) following the manufacturer's protocol. Real-time quantitative PCR (qPCR) was performed using GoTaq DNA polymerase (Promega) on the QuantStudio 5 Real-Time PCR System (Applied Biosystems). The relative expression of AZIN1 or ADAR1 (defined as ‘relative expression’) is given as 2^{-ΔC}^{_{T}} (ΔC_{T}=C_{T}(AZIN1/ADAR1)−C_{T}(β-actin)) and normalised to the relative expression that was detected in the corresponding control cells, which was defined as 1.0. Semi-quantitative PCR was done using the FastStart Taq kit (Roche) following the manufacturer's protocol. Purified PCR amplicons were identified by Sanger sequencing. ImageJ was used to calculate the percentage of A-to-I(G) editing. The percentage of editing is calculated as the area of “G” peak over the total area of “A” and “G” peaks. Sequences of primers are listed in Table 1.

TABLE 1
Sequences of primers used.

| Gene | | |
|---|---|---|
| Name | Primers | Remarks |
| FA | Forward: 5′→3′ | For minigene study |
| | CGGGATCCATTCATTTTCCCCTTTCCTGTTTT | |
| | ATTCC (SEQ ID NO: 10) | |
| | Reverse: 5′→3′ | |
| | CGGAATTCTCCAGCATCTTGCATCTCATACC | |
| | (SEQ ID NO: 11) | |
| FB | Forward: 5′→3′ | |
| | CGGGATCCATTCATTTTCCCCTTTCCTGTTTT | |
| | ATTCC (SEQ ID NO: 10) | |
| | Reverse: 5′→3′ | |
| | CGGAATTCGCTTAAGGGGTAGGACAAACTGG | |
| | T (SEQ ID NO: 12) | |
| FC | Forward: 5′→3′ | |
| | CGGGATCCGCAAGTTTTATCAGAAATATCAA | |
| | AACCTATTTGGCA (SEQ ID NO: 13) | |
| | Reverse: 5′→3′ | |
| | CGGAATTCGCTTAAGGGGTAGGACAAACTGG | |
| | T (SEQ ID NO: 12) | |
| FD | Forward: 5′→3′ | |
| | CGGGATCCAAATACAAGGAAGATGAGCCTCT | |
| | GTTTACAA (SEQ ID NO: 14) | |
| | Reverse: 5′→3′ | |
| | CGGAATTCGCTTAAGGGGTAGGACAAACTGG | |
| | T (SEQ ID NO: 12) | |
| FE | Forward: 5′→3′ | |
| | CGGGATCCAAATACAAGGAAGATGAGCCTCT | |
| | GTTTACAA (SEQ ID NO: 14) | |
| | Reverse: 5′→3′ | |
| | CGGAATTCTTAAGCTTCAGCGGAAAAGCTGT | |
| | C (SEQ ID NO: 15) | |
| FE-1 | Forward: 5′→3′ | |
| | CTGAGCCGAATTCAATCGATGGCCGCCATG | |
| | (SEQ ID NO: 16) | |
| | Reverse: 5′→3′ | |
| | GAATTCGGCTCAGCTGAATGCAAGAAGGCAC | |
| | AAAG (SEQ ID NO: 17) | |
| FE-2 | Forward: 5′→3′ | |
| | GAAGACAGCTGAAGCTTAAGAATTCAATCGA | |
| | TGGCC (SEQ ID NO: 18) | |
| | Reverse: 5′→3′ | |
| | TTCAGCTGTCTTCTTGGCTCAGCTGAATGCAA | |
| | G (SEQ ID NO: 19) | |
| FE-3 | Forward: 5′→3′ | |
| | CTCATTCAGTGCAGAAGCTTAAGAATTCAAT | |
| | CGATGGCCGC (SEQ ID NO: 20) | |
| | Reverse: 5′→3′ | |
| | TGCACTGAATGAGTCTTCTTGGCTCAGCTGA | |
| | ATGCAAGAA (SEQ ID NO: 21) | |
| HTR2C | Forward: 5′→3′ | |
| | CGGGATCCATCATGCACCTCTGC | |
| | GCTATATCG (SEQ ID NO: 22) | |
| | Reverse: 5′→3′ | |
| | CGGAATTCAGAACCCGATCAAACGCAAATGT | |
| | TACC (SEQ ID NO: 23) | |
| ADAR1 | Forward: 5′→3′ | For QPCR analysis |
| | GCTGAAGCTGGAAGCAAGAAAGTG (SEQ ID | |
| | NO: 24) | |
| | Reverse: 5′→3′ | |
| | CAGGGCCTTCTTTGGACAGGA (SEQ ID NO: | |
| | 25) | |
| AZIN1 | Forward: 5′→3′ | |
| | ATTGATGATGCAAACTACTCCGTTGG (SEQ ID | |
| | NO: 26) | |
| | Reverse: 5′→3′ | |
| | CTGGAGGTACACCCAACTCTTG (SEQ ID NO: | |
| | 27) | |
| β-actin | Forward: 5′→3′ | |
| | ACCCTGAAGTACCCCATCGA (SEQ ID NO: 28) | |
| | Reverse: 5′→3′ | |
| | CTCAAACATGATCTGGGTCATCT (SEQ ID NO: | |
| | 29) | |
| AZIN1 | Forward: 5′→3′ | For PCR & Sanger |
| (endogenous) | GCATTTACACTCGCAGTTAATATCATAGC | sequencing |
| | (SEQ ID NO: 30) | |
| | Reverse: 5′→3′ | |
| | AATGCAAGAAGGCACAAAGAAGAAG (SEQ | |
| | ID NO: 31) | |
| AZIN1 | Forward: 5′→3′ | |
| (minigene | CGGATTCCCCGTGCCAAGAGTGAC (SEQ ID | |
| derived) | NO: 32) | |
| | Reverse: 5′→3′ | |
| | GGCCATGGCGGCCATCGATT (SEQ ID NO: 33) | |
| AZIN1 (in | Forward: 5′→3′ | |
| vitro | TACAAGGAAGATGAGCCTCTGTTTACAA | |
| transcribed) | (SEQ ID NO: 34) | |
| | Reverse: 5′→3′ | |
| | TCCAGCATCTTGCATCTCATACC (SEQ ID NO: | |
| | 35) | |
| AZIN1 | Forward: 5′→3′ | |
| pre- | CCTACAGAAATACAAGGAAGATGAGCC (SEQ | |
| mRNA | ID NO: 36) | |
| | Reverse: 5′→3′ | |
| | TAAAATCACCTTACCAATCACTGAATGACA | |
| | (SEQ ID NO: 37) | |

Generation of Minigene Constructs

To clone AZIN1 sequences for the pRK7 or pcDNA3.1 minigene construction, placental DNA (Sigma-Aldrich) was used for PCR using PrimeSTAR Max DNA Polymerase (Takara), following the manufacturer's protocol. KAPA HiFi HotStart PCR Kit (KAPA Biosystems) was utilized to introduce internal deletion or point mutations. Sequences of primers used for cloning are listed in Table 1.

In Vitro RNA Editing Assay

First, forced overexpression of FLAG-tagged ADAR1 protein was performed by transfecting FLAG-ADAR1 plasmid into HEK293T cells. Cells were harvested 48 hours post transfection and lysed in lysis buffer containing 50 mM Tris-HCl pH 7.5 (Ambion), 150 mM NaCl (Ambion), 1 mM EDTA (Ambion), Triton-X100 (Sigma-Aldrich), and 1×COmplete™ EDTA-free Protease Inhibitor Cocktail (Roche). Anti-FLAG® M2 Magnetic Beads (Sigma-Aldrich) was used for immunoprecipitation of cell lysate to obtain FLAG-ADAR1 proteins. 100 μg/mL FLAG elution buffer was obtained by dissolving 3×FLAG peptide (Sigma) in Tris-buffered saline (TBS) containing 50 mM Tris-HCl pH 7.4 and 150 mM NaCl. FLAG elution buffer was used to elute the proteins from the magnetic beads. Eluant was stored at −80° C. until further use. In vitro transcription of the minigene constructs was done using RiboMAX™ Large Scale RNA Production System-SP6 (Promega) following manufacturer's protocol. Next, incubation of 5 μL FLAG-ADAR1 protein and purified RNA transcripts that were transcribed from the AZIN1 FE minigene was carried out at 37° C. for 3 h, followed by RNA cleaning-up using RNeasy Mini Kit (QIAGEN) and cDNA synthesis. To test the editing inhibitory effect of ASOs in vitro, ASOs were incubated with in vitro transcribed RNA transcripts prior to the addition of purified ADAR1 protein. PCR amplification was then conducted, and purified PCR products were sent for Sanger sequencing. Sequences of primers used for in vitro editing analysis are listed in Table 2.

RNA Electrophoretic Mobility Shift Assay (REMSA)—Binding of Each Oligo to the AZIN1 RNA Duplex Probe

The 86-nt AZIN1 RNA duplex probe was first transcribed in vitro using RiboMAX™ Large Scale RNA Production System-T7 (Promega) following the manufacturer's protocol. Next, 50 pmol of the RNA probe was incubated at 37° C. for 30 mins with rSAP (NEB) to dephosphorylate the RNA. EDTA (0.8 μL, 250 mM) was added and incubated at 65° C. for 20 mins to heat inactivate rSAP. The mixture was then incubated with 1 μL of 100 mM MgCl₂ with T4 PNK (NEB) and ATP, [γ-32P] (PerkinElmer) at 37° C. for 30 mins. The mixture was then heated to 95° C. to denature the duplex and was left to anneal slowly to room temperature. After which, 80 μL of distilled water was added and transferred to an Illustra MicroSpin G-25 Column (GE Healthcare) for purification. LightShift™ Chemiluminescent RNA EMSA Kit (Thermo Scientific) 10×REMSA Binding Buffer (100 mM HEPES pH7.3, 200 mM KCl, 10 mM MgCl₂, 10 mM DTT) was used to incubate the samples. Samples were mixed with 1 μL of RNA duplex (with a final concentration of 25 nM) and the respective oligonucleotides and incubated for 30 mins. Following that, FLAG-ADAR1 proteins were added when required and incubated further for 30 mins. TBE gel was pre-run before samples were added. The gel was dried and then exposed to either BioMax Light Film (Carestream, Sigma-Aldrich) and developed; or BAS Storage Phosphor Screen (GE Healthcare), imaged with Typhoon Trio Variable Mode Imager (GE Healthcare), and analysed with ImageQuant TL (GE Healthcare).

RNA Electrophoretic Mobility Shift Assay (REMSA)—Binding of Each PNA to the Truncated AZIN1 RNA Duplex Probe

Both strands (ECS-s and ES-s) were added together and slowly-cooled from 95° C. to room temperature to form the truncated RNA duplex before annealing of the PNAs at 40° C. for 10 mins. Both steps were carried out in an incubation buffer of 200 mM NaCl, 0.5 mM EDTA, and 20 mM HEPES, pH 7.5. After annealing the PNA, the samples were allowed to cool to room temperature before incubation at 4° C. overnight. The gel was run at constant voltage of 250 V for 5 hours in a running buffer of 1×TBE, pH 8.3. The gel was then stained in ethidium bromide for 30 mins before it is imaged using the Typhoon Trio Variable Mode Imager. Sequences of probes are listed in Table 2.

TABLE 2
Sequences of probes used.

| Probe | Length | Sequence | Remarks |
|---|---|---|---|
| AZIN | 86-nt | 5′- | This probe was used for REMSA |
| duplex | | UGAGCUUGAUCAAAUUGUG | experiments to test the binding |
| probe | | GAAAGCUGUCUUCUUCCUG | ability of each oligo. |
| | | AGCUAGCUGAGCCAAGAAG | |
| | | ACAGCUUUUCCGCUGAAGC | |
| | | UUAAACAGGC-3′ (SEQ ID | |
| | | NO: 38) | |
| Truncated | 21-nt | ECS-s: 5′- | This probe was used for REMSA |
| duplex | | CAAGAAGACAGCUUUUCCG | experiments to test the binding |
| probe | | CU-3′ (Editing site | ability of each PNA. |
| | | complementary sequence-short, | |
| | | ECS-s) (SEQ ID NO: 39) | |
| AZIN1 | 20-nt | ES-s: 5′- | |
| | | UGUGGAAAGCUGUCUUCUU | |
| | | C-3′ (Editing site sequence- | |
| | | short, ES-s) (SEQ ID NO: 40) | |

ASO Treatment

All ASOs were purchased from Integrated DNA Technologies (IDT). PNAs (ASP1, and DSP1 and DSP2) were synthesized and purified according to the protocol reported previously (Toh, D. K., Patil, K. M. & Chen, G. Sequence-specific and Selective Recognition of Double-stranded RNAs over Single-stranded RNAs by Chemically Modified Peptide Nucleic Acids. J Vis Exp, doi:10.3791/56221 (2017)). Cells were seeded the day before treatment to achieve 80% confluency on the day of treatment. Cells were then treated (transfected) with ASOs that were diluted in Opti-MEM to the desired concentration by Lipofectamine 2000. The subsequent analysis was conducted 48 hours post the treatment of ASO. Three independent experiments were carried out, each with three technical replicates conducted.

Cell Viability Assay

The CellTiter-Glo® Luminescent Cell Viability (CTG) Assay (Promega) was used to measure cell viability after cells were treated with ASOs. Cells were seeded and treated in 96-well clear flat bottom plates (Corning) for 2 days prior to the addition of the CTG assay reagent. A total of 100 μL of cell lysate was transferred to 96-well white flat-bottom plate (Corning). GloMax® Discover Microplate Reader (Promega) was used to read the intensity of luminescence.

Western Blot Analysis

Protein lysates were prepared with RIPA buffer (Sigma) supplemented with 1× cOmplete EDTA free protease inhibitor cocktail (Roche) and quantified using Bradford assay (Bio-Rad). Protein lysates were then separated by 8-10% SDS-PAGE followed by incubation with primary antibodies (1:1000 dilution) overnight at 4° C. and incubation with secondary antibodies (1:10,000 dilution) at room temperature for 1 hour. Primary antibodies used are anti-ADAR1 (Abcam, ab88574), anti-AZIN1 (Proteintech, 11548-1-AP), anti-GAPDH (Santa Cruz Biotechnology, sc-59540), anti-ODC (Abcam, ab66067), and anti-CCND1 (Cell signalling technology, 2978).

Foci Formation Assay

For foci formation assay, cells were seeded to obtain 80% confluency prior to the treatment of ASO. Cells were stained using crystal violet (Sigma-Aldrich) 48 hours after treatment.

Cell Cycle Analysis by PI Staining and FACS

Cells were treated with ASOs for 48 hours prior to the cell cycle analysis. After the treatment, cells were fixed with 70% ethanol in −20° C. overnight. After washing with phosphate-buffered saline (PBS; 10 mM phosphate, 137 mM NaCl and 2.7 mM KCl, cells were resuspended in 1 mL of staining solution containing 200 μL of 1 mg/mL PI (Invitrogen) and 20 μL of 10 mg/mL RNase A (Thermo Scientific), and incubated for 1 hour at 37° C. Stained cells were analyzed on the LSRII (BD Biosciences) and the results were analyzed on FACSDiva Software (BD Biosciences).

Loading of ASO into RBCEVs

Blood samples were obtained from healthy donors by Hong Kong Red Cross and EVs were produced from RBCs according to the established protocol (Usman, W. M. et al. Efficient RNA drug delivery using red blood cell extracellular vesicles. Nat Commun 9, 2359, doi:10.1038/s41467-018-04791-8 (2018)). ASOs were loaded into extracellular vesicles (EVs) derived from red blood cells (RBCEVs) at a ratio of 1 to 50, using ExoFect transfection reagent (System BioSciences) according to the manufacturer's protocol. RBCEVs were washed twice with PBS at 21,000×g for 30 mins at 4° C. to remove the free ASOs and transfection reagent.

Labelling RBCEVs with CFSE

A total of 200 μg of ASO-loaded RBCEVs were incubated with 400 μL of 10 μM CFSE at 37° C. for 2 hours. A total of 0.5 ml of CFSE-labelled RBCEVs was loaded onto a prepacked qEV-original size exclusion chromatographic column (Izon Science, New Zealand) and eluted with PBS in 40 fractions (0.5 ml/fraction). Fractions 7 to 11 were combined and centrifuged at 21,000×g for 30 mins at 4° C. The supernatant was removed, and the RBCEV pellet was washed twice with PBS, resuspended and quantified using a Nanodrop spectrophotometer (Thermo Fisher).

Fluorescence Imaging

Cells were cultured on coverslips for 24 hours and treated with CFSE-labelled ASO3.2-RBCEVs. At 48 hours post treatment, cells were washed with PBS before fixation with methanol for 10 mins at room temperature. Fixed cells were washed with PBS thrice 5 mins each. The coverslips were mounted onto slides using SlowFade Gold antifade mountant with DAPI (Thermo Fisher Scientific) and viewed under Zeiss Axio Imager M2 microscope.

In Vivo Tumorigenicity Assays

Pre-Treatment Model

KYSE510 cells were pre-treated with 100 nM of ASO3.2 and ASO-ctl using Lipofectamine 2000 (Invitrogen) for 48 hours, followed by the subcutaneous injection of 4×106 of pre-treated cells into left and right dorsal flanks of 4- to 6-week-old NOD scid gamma (NSG) mice (n=6 mice per group). Tumor growth was monitored by measuring tumor length (L) and width (W) at indicated time points. Tumor volume was calculated by the formula V=0.5×L×W2. All animal experiments were approved by and performed in accordance with the Institutional Animal Care and Use Committees of National University of Singapore (NUS, Singapore).

Intratumoral Injection Model

A total of 2×10⁶ of KYSE510 cells was injected subcutaneously to the right and left flanks of 4-6 weeks NSG mice for tumor development. When tumors were visible (approximately 1 mm in diameter), mice were divided into 2 groups (6 mice per group) for multiple intratumoral (i.t.) injection of ASO-loaded RBCEVs (Group 1: RBCEVs-based delivery) or naked ASO (Group 1: naked ASO) every 4 days for 7 weeks. For each injection of ASO-loaded RECEVs per tumor, a total of 1 g ASO was loaded into 50 μg of RBCEVs and resuspended in 20 μL of PBS. For each injection of naked ASO per tumor, a total of 13.5 μg of ASOs (ASO-ctl or ASO3.2) was dissolved in 20 μL of PBS. Tumor growth was monitored by measuring tumor length (L) and width (W) at indicated time points. Tumor volume was calculated by the formula V=0.5×L×W2. All animal experiments were approved by and performed in accordance with the Institutional Animal Care and Use Committees of National University of Singapore (NUS, Singapore).

Statistical Analysis

Unpaired, two-tailed Student's t-test was used for statistical analysis of changes in cell viability and tumor growth rate, between the control and treatment group. For all figures: *, P<0.05; **, P<0.01; ***, P<0.001.

Results

An 8-Nt Sequence at 3′ End of Exon 12 is the Core ECS and Indispensable for AZIN1 Editing

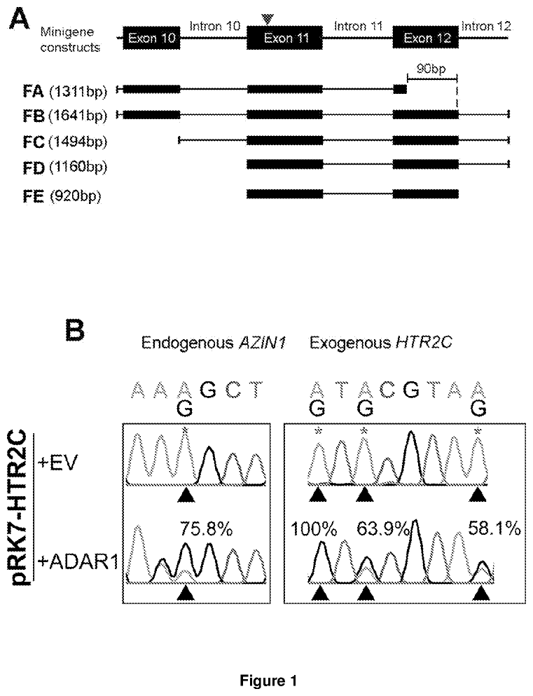

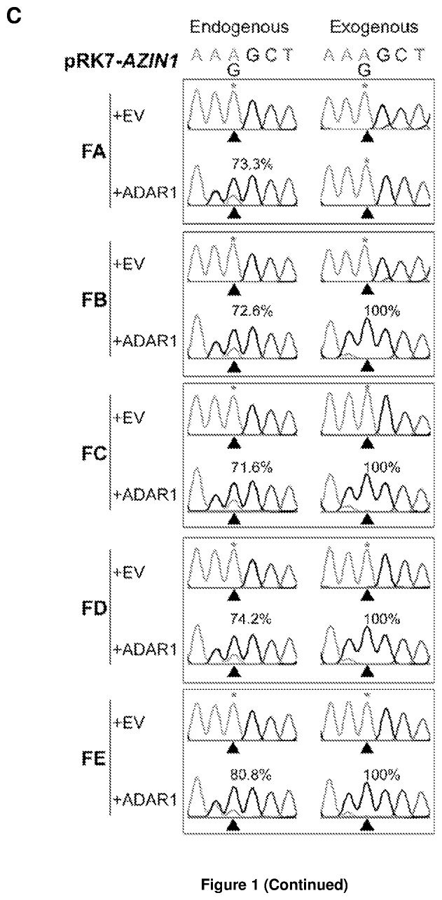

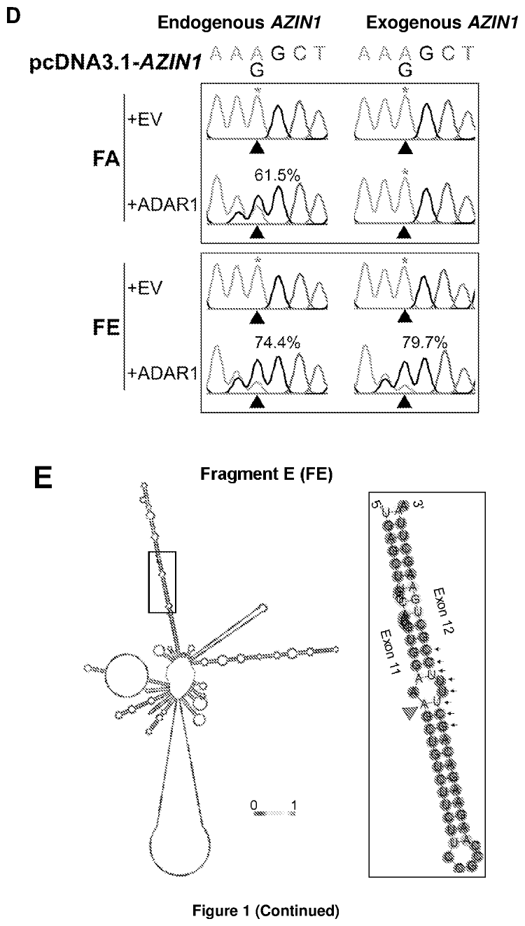

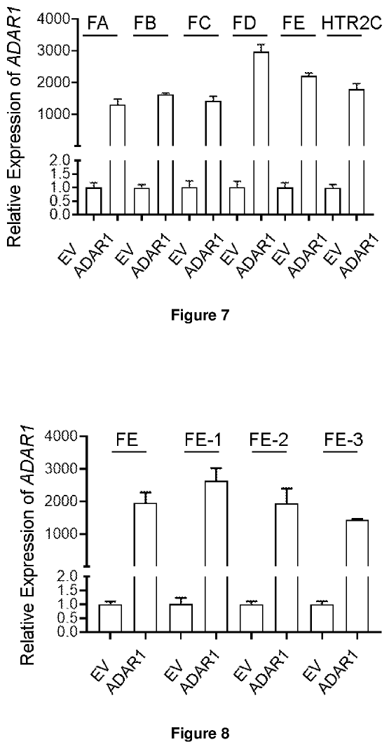

Uncovering the ECS of AZIN1 transcript would help to decipher the precise dsRNA structure which is essential for AZIN1 editing. To this end, AZIN1 minigene constructs were generated by inserting fragments of different length covering the edited exon 11 and flanking exons and introns, into either pRK7 or pcDNA3.1 vector (FIG. 1A). Each of the AZIN1 minigene constructs was co-transfected with ADAR1 expression construct or empty vector into HEK293T cells, followed by editing analysis of endogenous AZIN1 and exogenous transcripts which were transcribed from AZIN1 minigenes. It was first examined whether ADAR1 could work effectively on exogenous transcripts transcribed from pRK7-based minigene system. HTR2C, which is a well-characterized editing target with its dsRNA structure well delineated in many studies, was used to generate HTR2C minigene as a positive control. Upon the co-transfection of HTR2C minigene and ADAR1, three known A-to-I editing sites were detected in exogenous HRT2C transcripts (FIG. 1B), supporting the feasibility of using the pRK7 minigene system in this study. Besides, approximately 75.8% of endogenous AZIN1 was edited, indicative of successful ADAR1 overexpression (FIG. 1B and FIG. 7). Amongst all AZIN1 minigenes, only AZIN1 transcripts transcribed from the minigene containing fragment A (FA), which lacks 90-bp sequence at 3′ end of exon 12, was unable to be edited (FIG. 1A, 1C). This observation could be reproduced by using pcDNA3.1-based minigene, ruling out the possibility of artefacts of the pRK7 minigene system (FIG. 1D). All these findings suggested the ECS of AZIN1 is most likely at the 3′ end of exon 12.

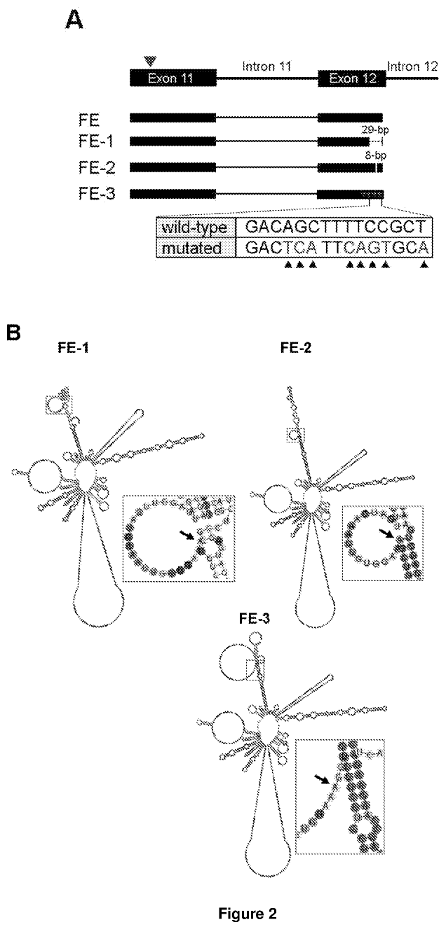

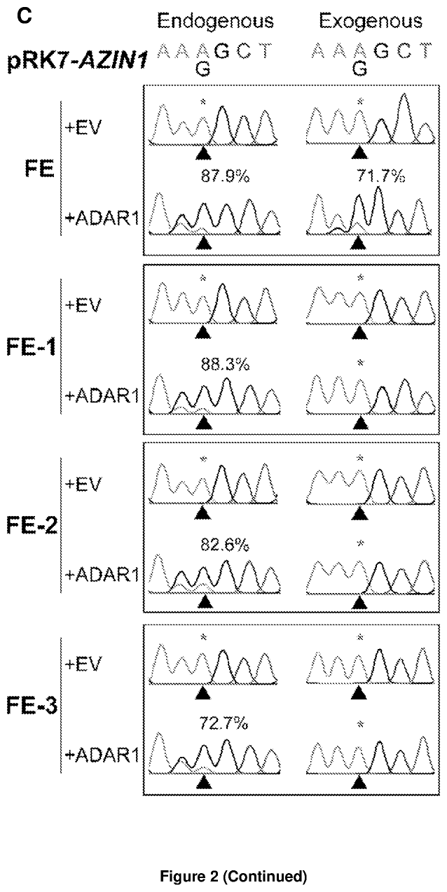

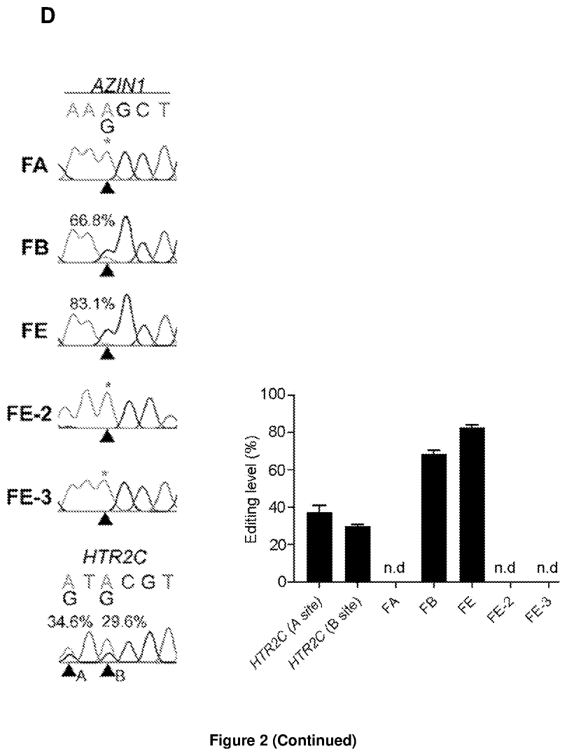

To precisely locate the ECS, RNA sequences corresponding to fragment E (FE) was subjected to secondary structure prediction by RNAFold30. As expected, 3′end of exon 12 forms dsRNA with the edited sequence (FIG. 1E). FE minigene was utilized to generate three additional minigenes by deleting 29-bp sequence at 3′ end of exon 12 (FE-1), introducing an 8-bp internal deletion (FE-2), or point mutations (FE-3) in the sequence directly opposite to the editing region (FIG. 2A). Secondary structure prediction showed that both deletion and mutations could dramatically alter the secondary structure (FIG. 2B). Using the same strategy, it was observed that the transcripts transcribed from FE-1, 2 and 3 minigenes failed to be edited upon ADAR1 overexpression (FIG. 2C and FIG. 8). Further, in vitro RNA editing analysis was conducted and it was found that in the presence of purified ADAR1 protein, in vitro transcribed AZIN1 transcripts from FB or FE, but not FA, FE-2 and FE-3 minigenes, were edited at expected editing site (FIG. 2D). All these data strongly indicate that an 8-nt sequence (5′-GCUUUUCC-3′) at the 3′end of exon 12 is the core ECS and indispensable for dsRNA formation and AZIN1 editing.

Identifying ASOs with Pronounced In Vitro Editing Inhibitory Effects

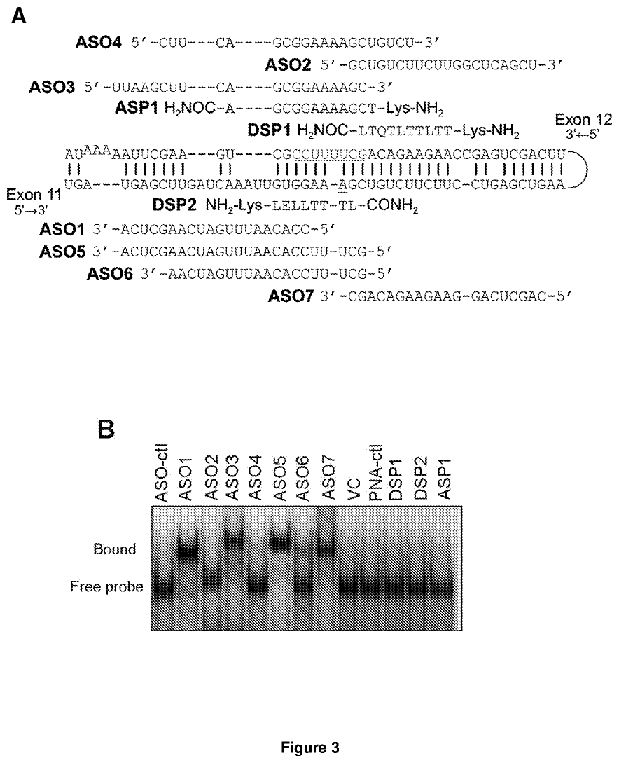

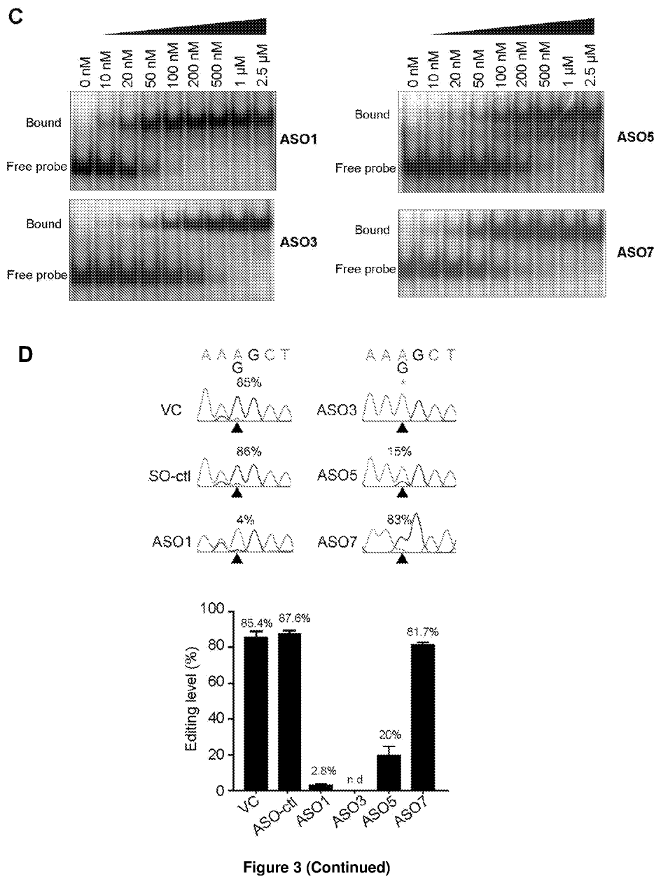

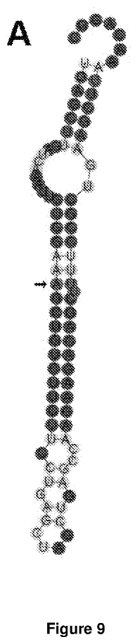

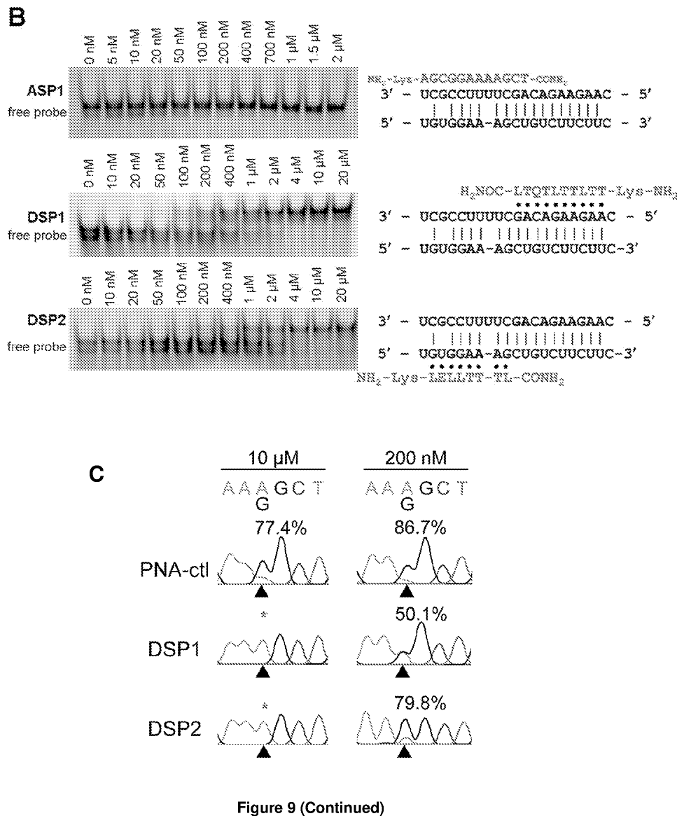

Based on the elucidation of the AZIN1 dsRNA structure, seven entirely 2′-O-Me-modified ASOs (ASO1-ASO7) and three PNAs including an antisense PNA (ASP1) and 2 dsRNA-binding PNAs (DSP1 and DSP2) which can form triplex with AZIN1 dsRNA, were designed, synthesized and evaluated for their binding abilities to the AZIN1 dsRNA (Table 3 and FIG. 3A). Each oligo was subjected to the RNA electrophoretic mobility shift assay (REMSA), in order to examine their binding capability to the 32P-labelled AZIN1 RNA duplex probe (FIG. 9A). A strong band shift was observed in the presence of ASO1, ASO3, ASO5 and ASO7, while very weak or no band shift was detected upon the addition of ASO2, ASO4, ASO6 and all three PNAs (FIG. 3B). Notably, with increasing amounts of ASO1, ASO3, ASO5 or ASO7 added, a dose-dependent increase in the AZIN1 duplex binding was detected, which further confirmed the binding capability (with sub-micromolar affinity) of these ASOs to AZIN1 (FIG. 3C). Since PNAs are shorter than ASOs, further testing using a shortened AZIN1 duplex confirmed that the 12-mer ASP1 was incapable of binding to the shortened AZIN1 RNA duplex, while DSP1 and DSP2 could bind through PNA-dsRNA triplex formation with a modest binding affinity (micromolar; FIG. 9B).

TABLE 3
Characteristics of ASOs and PNAs used.

| | | Chemical | | | |
|---|---|---|---|---|---|
| ASO | Length | Modification | Sequence | MW | Remarks |
| ASO1 | 20- | Full 2′-O-Me | 5′- | 6575.4 | targets |
| | mer | modification | ^{m}C^{m}C^{m}A^{m}C^{m}A^{m}A^{m}U^{m}U^{m}U^{m}G^{m} | | the |
| | | | A^{m}U^{m}C^{m}A^{m}A^{m}G^{m}C^{m}U^{m}C^{m}A-3′ | | editing |
| | | | (SEQ ID NO: 41) | | strand |
| ASO1.1 | 20- | Full 2′-O-Me | 5′- | 6880.5 | targets |
| | mer | and PS | ^{m}C*^{m}C*^{m}A*^{m}C*^{m}A*^{m}A*^{m}U*^{m} | | the |
| | | modifications | U*^{m}U*^{m}G*^{m}A*U*^{m}C*^{m}A*^{m}A | | editing |
| | | | *^{m}G*^{m}C*^{m}U*^{m}C*^{m}A-3′ (SEQ | | strand |
| | | | ID NO: 42) | | |
| ASO1.2 | 20- | Full 2′-O-Me | 5′- | 6655.7 | targets |
| | mer | and partial PS | ^{m}C^{m}C^{m}A^{m}C^{m}A^{m}A^{m}U^{m}U^{m}U^{m}G^{m} | | the |
| | | modifications | A^{m}U^{m}C^{m}A^{m}A*^{m}G*^{m}C*^{m}U*^{m}C* | | editing |
| | | of 5 | ^{m}A-3′ (SEQ ID NO: 43) | | strand |
| | | nucleotides at | | | |
| | | 3′-end | | | |
| ASO1.3 | 20- | Full 2′-O-Me | 5′- | 6655.7 | targets |
| | mer | and partial PS | ^{m}C*^{m}C^{m}A*^{m}C^{m}A*^{m}A^{m}U^{m}U^{m} | | the |
| | | modifications | U^{m}G^{m}A^{m}U^{m}C^{m}A^{m}A^{m}G^{m}C^{m}U^{m}C | | editing |
| | | of 5 | ^{m}A-3′ (SEQ ID NO: 44) | | strand |
| | | nucleotides at | | | |
| | | 5′-end | | | |
| ASO2 | 20- | Full 2′-O-Me | 5′- | 6554.3 | targets |
| | mer | modification | ^{m}G^{m}C^{m}U^{m}G^{m}U^{m}C^{m}U^{m}U^{m}C^{m}U^{m} | | the ECS |
| | | | U^{m}G^{m}G^{m}C^{m}U^{m}C^{m}A^{m}G^{m}C^{m}U-3′ | | |
| | | | (SEQ ID NO: 45) | | |
| ASO3 | 20- | Full 2′-O-Me | 5′- | 6694.5 | targets |
| | mer | modification | ^{m}U^{m}U^{m}A^{m}A^{m}G^{m}C^{m}U^{m}U^{m}C^{m}A^{m} | | the ECS |
| | | | G^{m}C^{m}G^{m}G^{m}A^{m}A^{m}A^{m}A^{m}G^{m}C-3′ | | |
| | | | (SEQ ID NO: 6) | | |
| ASO3.1 | 20- | Full 2′-O-Me | 5′- | 6999.6 | targets |
| | mer | and PS | ^{m}U*^{m}U*^{m}A*^{m}A*^{m}G*^{m}C*^{m}U*^{m} | | the ECS |
| | | modifications | U*^{m}C*^{m}A*^{m}G*^{m}C*^{m}G*^{m}G*^{m}A | | |
| | | | ^{m}*A*^{m}A*^{m}A*^{m}G*^{m}C-3′ (SEQ | | |
| | | | ID NO: 7) | | |
| ASO3.2 | 20- | Full 2′-O-Me | 5′- | 6774.8 | targets |
| | mer | and partial PS | ^{m}U*^{m}U*^{m}A*^{m}A*^{m}G*^{m}C^{m}U^{m}U^{m} | | the ECS |
| | | modifications | C^{m}A^{m}G^{m}C^{m}G^{m}G^{m}A^{m}A^{m}A^{m}A^{m}G | | |
| | | of 5 | ^{m}C-3′ (SEQ ID NO: 8) | | |
| | | nucleotides at | | | |
| | | 5′-end | | | |
| ASO3.3 | 20- | Full 2′-O-Me | 5′- | 6774.8 | targets |
| | mer | and partial PS | ^{m}U^{m}U^{m}A^{m}A^{m}G^{m}C^{m}U^{m}U^{m}C^{m}A^{m} | | the ECS |
| | | modifications | G^{m}C^{m}G^{m}G^{m}A*^{m}A*^{m}A*^{m}A*^{m}G* | | |
| | | of 5 | ^{m}C-3′ (SEQ ID NO: 9) | | |
| | | nucleotides at | | | |
| | | 3′-end | | | |
| ASO4 | 20- | Full 2′-O-Me | 5′- | 6647.4 | targets |
| | mer | modification | ^{m}C^{m}U^{m}U^{m}C^{m}A^{m}G^{m}C^{m}G^{m}G^{m}A^{m} | | the ECS |
| | | | A^{m}A^{m}A^{m}G^{m}C^{m}U^{m}G^{m}U^{m}C^{m}U-3′ | | |
| | | | (SEQ ID NO: 46) | | |
| ASO5 | 25- | Full 2′-O-Me | 5′- | 8214.4 | targets |
| | mer | modification | ^{m}G^{m}C^{m}U^{m}U^{m}U^{m}C^{m}C^{m}A^{m}C^{m}A^{m} | | the |
| | | | A^{m}U^{m}U^{m}U^{m}G^{m}A^{m}U^{m}C^{m}A^{m}A^{m}G | | editing |
| | | | ^{m}C^{m}U^{m}C^{m}A-3′ (SEQ ID NO: | | strand |
| | | | 47) | | |
| ASO6 | 20- | Full 2′-O-Me | 5′- | 6553.3 | targets |
| | mer | modification | ^{m}G^{m}C^{m}U^{m}U^{m}U^{m}C^{m}C^{m}A^{m}C^{m}A^{m} | | the |
| | | | A^{m}U^{m}U^{m}U^{m}G^{m}A^{m}U^{m}C^{m}A^{m}A-3′ | | editing |
| | | | (SEQ ID NO: 48) | | strand |
| ASO7 | 20- | Full 2′-O-Me | 5′- | 6755.6 | targets |
| | mer | modification | ^{m}G^{m}C^{m}U^{m}U^{m}U^{m}C^{m}C^{m}A^{m}C^{m}A^{m} | | the |
| | | | A^{m}G^{m}A^{m}A^{m}G^{m}A^{m}C^{m}A^{m}G^{m}C-3′ | | editing |
| | | | (SEQ ID NO: 49) | | strand; |
| | | | | | 20-nt |
| | | | | | immediately |
| | | | | | downstream |
| | | | | | of |
| | | | | | editing |
| | | | | | site |
| DSP1 | 10- | dsRNA- | NH₂-Lys-TTLTTLTQTL- | 2895.2 | complement |
| | mer | binding PNA, | CONH₂ (SEQ ID NO: 50) | | with the |
| | | modified | | | duplex |
| | | bases L and Q | | | containing |
| | | | | | the |
| | | | | | segment |
| | | | | | AAGAA |
| | | | | | GACAG |
| DSP2 | 8- | dsRNA- | NH₂-Lys-LELLTTTL-CONH₂ | 2276.9 | complement |
| | mer | binding PNA, | | | with the |
| | | modified | | | duplex |
| | | bases L and E | | | containing |
| | | | | | the |
| | | | | | segment |
| | | | | | GUGGA |
| | | | | | AAG |
| ASP1 | 12- | SsRNA- | NH₂-Lys-AGCGGAAAAGCT- | 3453.4 | |
| | mer | binding | CONH₂ (SEQ ID NO: 51) | | |
| | | (antisense) | | | |
| | | PNA, | | | |
| | | unmodified | | | |
| | | bases | | | |
| ASO-Ctl | 20- | Full 2′-O-Me | 5′- | 2362.0 | |
| | mer | and partial PS | ^{m}C^{m}G^{m}U^{m}G^{m}U^{m}G^{m}U^{m}U^{m}C^{m}U^{m} | | |
| | | modifications | A^{m}C^{m}G^{m}C^{m}U*^{m}C*^{m}U*^{m}G*^{m}G* | | |
| | | of 5 | ^{m}U-3′ (SEQ ID NO: 52) | | |
| | | nucleotides at | | | |
| | | 3′-end | | | |
| PNA-Ctl | 8- | dsRNA- | NH₂-Lys-TLTLTTTL-CONH₂ | 2278.9 | |
| | mer | binding PNA, | | | |
| | | modified | | | |
| | | bases L and Q | | | |

m, 2′-O-Me modified;
*, PS modifier id PNAs used.

Next, to examine whether the binding of each oligo to the AZIN1 duplex is sufficient to inhibit AZIN1 editing, ASO1, 3, 5 and 7 were subjected to in vitro RNA editing assays. With the addition of ASO3, AZIN1 editing was completely abolished. Compared to ASO3, ASO1 was slightly less effective but dramatically repressed editing from 87.6% to 2.8%; while for ASO5 or ASO7, they demonstrated low or no inhibition of AZIN1 editing, respectively (FIG. 3D). Of note, ASO5 is a 25-mer ASO with the addition of 5 nucleotides (GCUUU) on to the 5′end of ASO1 (FIG. 3A and Table 3). Although ASO5 could target the editing site due to this extension, it failed to improve or maintain the editing inhibitory effect of ASO1, consistent with the fact that ASO5 has a slightly weakened binding compared to ASO1 (FIG. 3C), probably because the bases pairs involving the edited sequence (AAAGC) (potentially 3 A-U and 2 G-C pairs) are relatively more stable and difficult to be invaded by ASOs (FIG. 3A). This was also supported by the observation that ASO6, which shares the same sequence with ASO5 except 5-nt shorter than ASO5 at its 3′end (FIG. 3A and Table 3), was largely incapable of binding to AZIN1 duplex (FIG. 3C). In addition, DSP1 and DSP2 were able to abolish AZIN1 editing at a concentration of 10 M, but their editing inhibitory effects were dramatically attenuated at 200 nM (FIG. 9C), suggesting that PNA-dsRNA triplex formation may not be as effective as the conventional Watson-Crick base pairing for editing inhibition. All these data suggested that ASO1 which targets the editing region flanking the editing site, and ASO3 which targets the ECS, could bind to AZIN1 transcripts and abolish or substantially inhibit AZIN1 editing in vitro, at nanomolar concentrations.

ECS-Targeting ASOs Dramatically Inhibit AZIN1 Editing in Cancer Cells

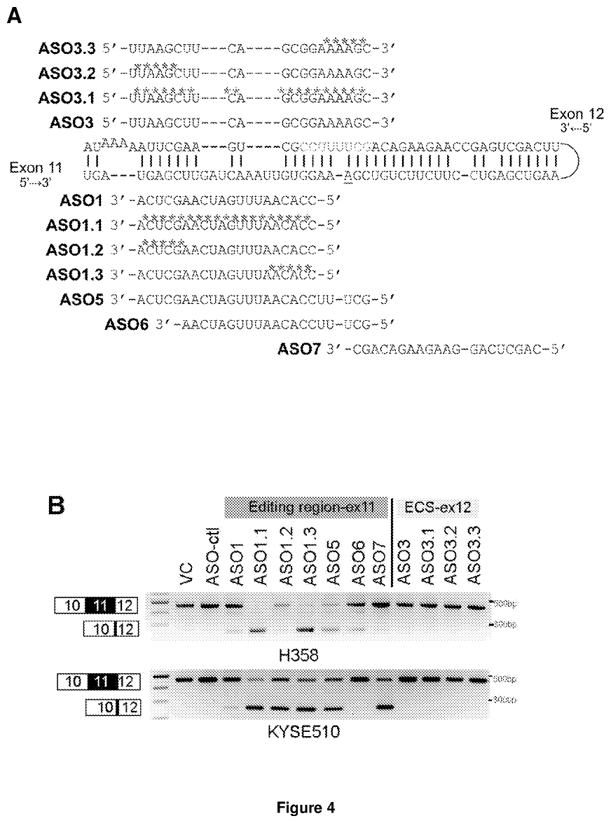

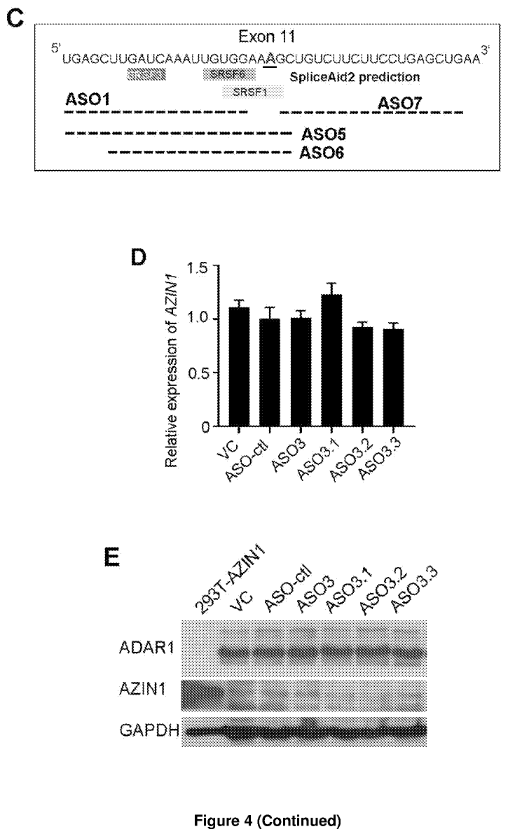

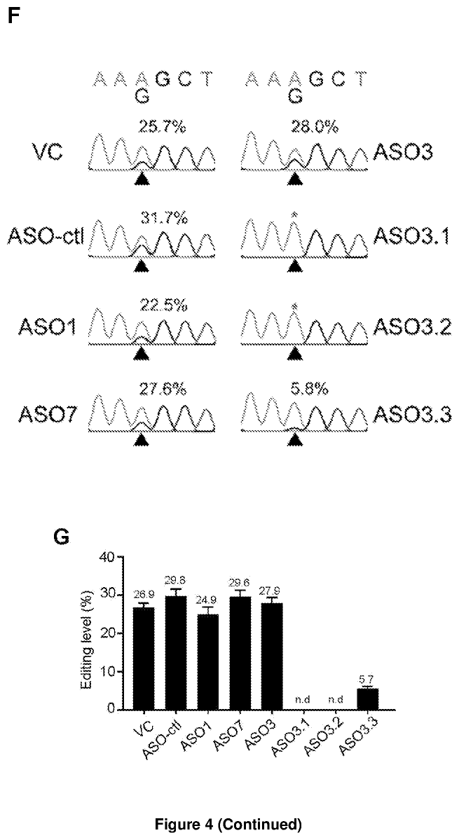

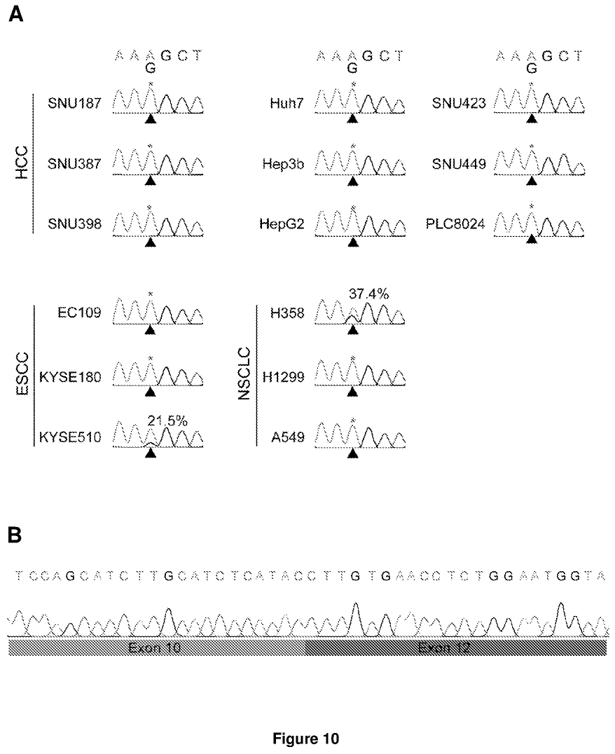

Currently, the most widely used chemistries for pre-mRNA binding and splicing modulation are the PS backbone with 2′-O-Me/2′-O-MOE/LNA or PMO, fully modified over the entire oligo length. Their stability, nuclease resistance, target affinity and inability to trigger RNase H/RNAi response make them the ideal tools for pre-mRNA binding, splicing and probably RNA editing. Therefore, 2′-O-Me-modified ASO1 and ASO3 were further fully or partially modified with PS (ASO1.1, 1.2, 1.3 and ASO3.1, 3.2 and 3.3; FIG. 4A and Table 3) and included in this study. The basal editing level of AZIN1 among 9 HCC, 3 ESCC and 3 NSCLC cell lines was screened, and it was found that AZIN1 editing was only detected in an ESCC line KYSE510 and a NSCLC line H358 (FIG. 10A). KYSE510 and H358 cells were next treated with each of chemically modified ASOs. Unexpectedly, all 7 ASOs (ASO1, 1.1, 1.2, 1.3, 5, 6, and 7) targeting the editing region led to skipping of exon 11 (FIG. 4B and FIG. 10B), possibly due to the existence of splicing factor binding sites at the editing region predicted by SpliceAid231 (FIG. 4C). Notably, of the 3 ECS-targeting ASOs, ASO3.1 and ASO3.2 completely abolished AZIN1 editing and ASO3.3 dramatically inhibited editing, without affecting splicing and expression of AZIN1 at both mRNA and protein levels (FIG. 4D-G). It is likely that additional PS modification in 2′-O-Me-modified ASO3 increases the chemical stability, resulting in improved editing inhibition efficacies of ASO3.1, ASO3.2 and ASO3.3 in cells. The above findings suggested that the editing region at exon 11 of AZIN1 transcript is non-targetable, and only ASOs targeting the ECS could effectively inhibit AZIN1 editing in cancer cells.

ASO3.2 Specifically Inhibits G1/S Transition and Cancer Cell Viability

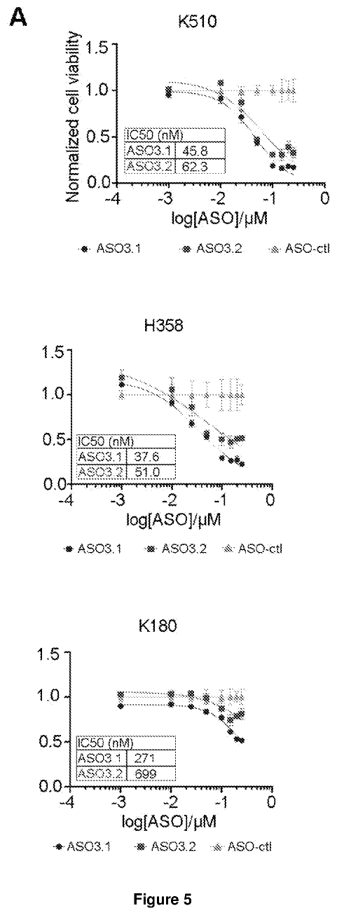

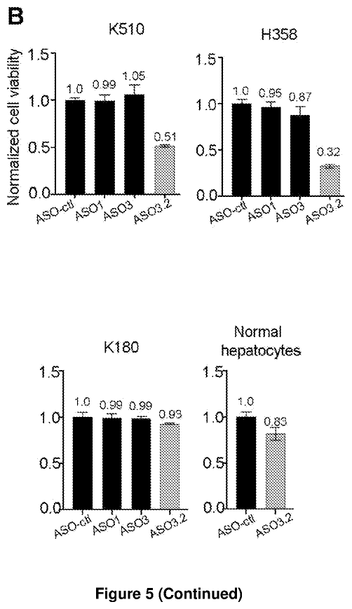

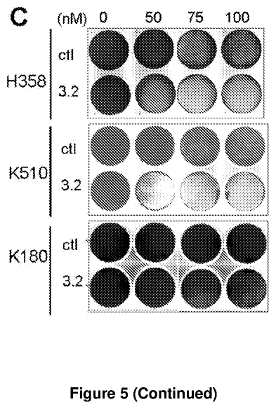

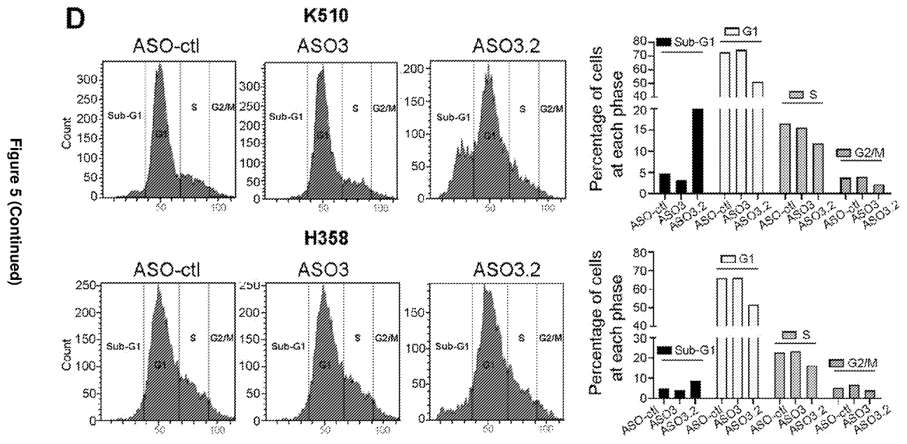

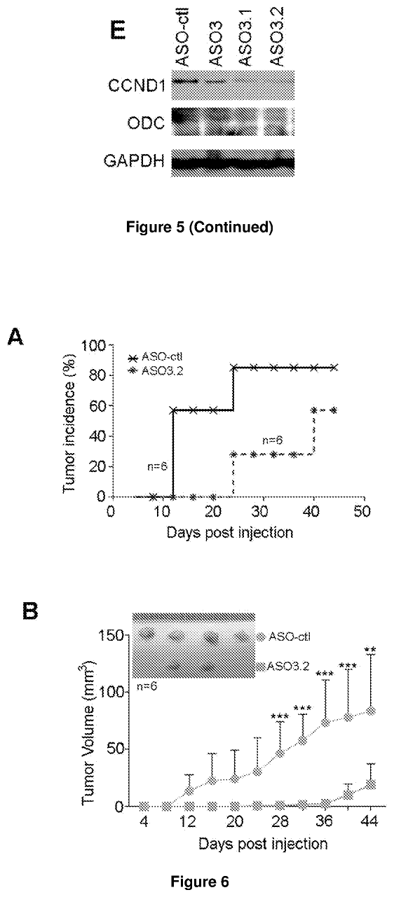

It was next studied whether the most potent ASOs ASO3.1 and ASO3.2 specifically inhibit cancer cell viability through repressing AZIN1 editing. To this end, in addition to KYSE510 and H358, an AZIN1 editing-null ESCC cell line KYSE180 was also included in the study. Three cell lines were treated with increasing concentrations of ASO3.1, ASO3.2 or ASO-ctl by lipofectamine transfection, followed by cell viability analyses. It was observed that both ASO3.1 and ASO3.2 dramatically inhibited cell viability of KYSE510 and H358 with low IC50 values (ASO3.1: K510:45.8 nM and H358: 37.6 nM; ASO3.2: K510: 62.3 nM and H358: 51.0 nM); while they demonstrated much less inhibitory effects on cell viability of KYSE180 (ASO3.1: 271 nM; ASO3.2: 699 nM) (FIG. 5A). Of note, KYSE180 demonstrated approximately 2.6-fold lower IC50 sensitivity to ASO3.2 than ASO3.1, implying that ASO3.2 most likely confers a higher specificity to repress editing and cancer cell viability than ASO3.1 (FIG. 5A). To further confirm the specific inhibitory effect of ASO3.2, three cell lines were subjected to cell viability and foci formation assays after the treatment with ASO3.2 at low dosage. As a result, ASO3.2 only inhibited cell viability of KYSE510 and H358, but not KYSE180 (FIG. 5B, C). In addition to cancer cells, normal human hepatocytes isolated from the perfused livers of humanized mice were also treated with ASO3.2 or ASO-ctl (FIG. 5B). It was found that normal hepatocytes were not sensitive to ASO3.2 treatment. All these data supported that ASO3.2 could specifically inhibit cell viability of cancer cells which express edited AZIN1^{S367G}. Cell cycle analysis showed that upon treatment of ASO3.2, KYSE510 and H358 cells demonstrated an obvious attenuation of GUS transition and a dramatic increase in the percentage of sub-G1 phase (apoptotic cells) when compared to cells treated with ASO-ctl or ASO3 (FIG. 5D). Further, a reduction in CCND1 and ODC protein expression supported the GUS arrest induced by ASO3.2 (FIG. 5E). Altogether, ASO3.2 could specifically inhibit AZIN1 editing in cancer cells, leading to a decline in CCND1 expression and the resultant GUS arrest and reduced cancer cell viability.

ASO3.2 Effectively Inhibits Tumor Incidence and Growth In Vivo

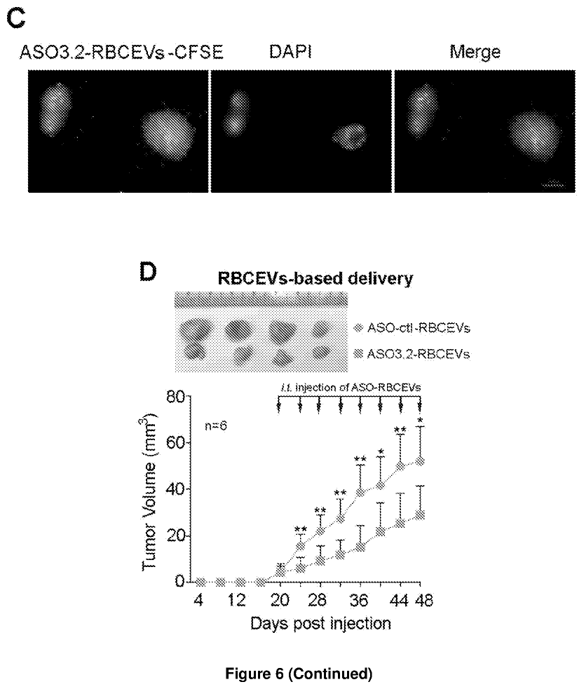

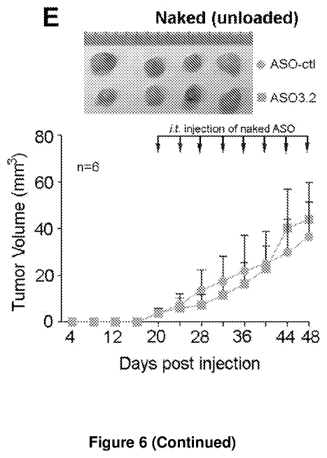

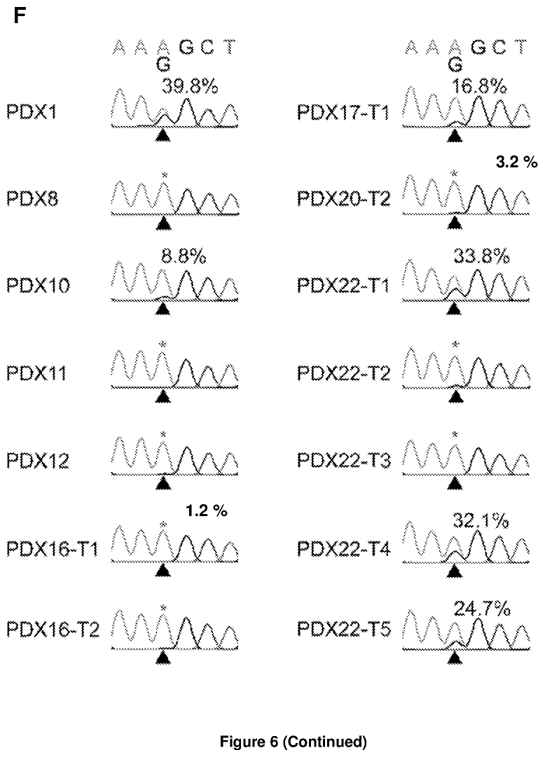

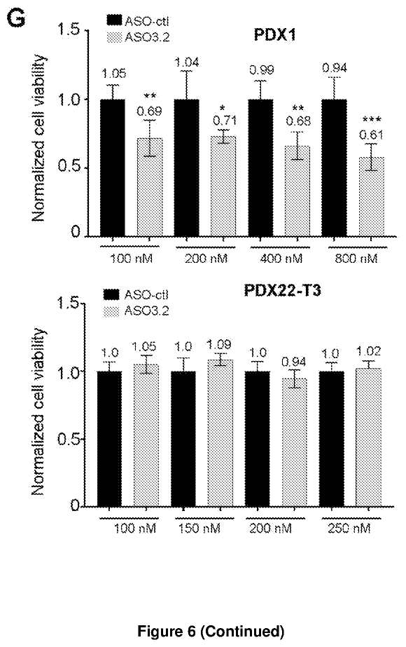

The effect of ASO3.2 in tumor incidence and growth was investigated using 2 xenograft tumor models. KYSE510 cells were pre-treated with ASO-ctl or ASO3.2 using lipofectamine transfection, followed by subcutaneous injection into two dorsal flanks of mice to compare their tumor incidence and growth rates. Tumor incidence rate of the ASO3.2 pre-treated group was markedly lower than that of the ASO-ctl pre-treated group (FIG. 6A). Moreover, during a 6-week observation period, tumors derived from ASO-ctl pre-treated cells grew significantly faster than tumors derived from ASO3.2 pre-treated cells (FIG. 6B). In addition to the pre-treated model, the effect of ASO3.2 on tumor growth was also investigated through intratumoral injection. Extracellular vesicles (EVs) are small membrane vesicles released from different types of cells and increasingly being recognized as natural RNA carriers and novel drug delivery vehicles. After entry into the cell, the cargo will be released from the EVs, and ASOs will be transported to the nucleus. To deliver ASOs into tumor cells, ASO3.2 or ASO-ctl was loaded into EVs derived from human red blood cells (RBCEVs), an ideal source of EVs with promising properties for RNA drug delivery. To test the cellular uptake of ASO3.2-RBCEVs, ASO3.2-RBCEVs were labelled with carboxyfluorescein succinimidyl ester (CFSE), which fluoresces only in the presence of esterase when they are either loaded into RBCEVs or internalized into cells. It was found that the majority of ASO3.2-RBCEVs could enter the cells (FIG. 6C). Next, KYSE510 cells were injected into two dorsal flanks of mice subcutaneously for tumor development. When tumors were visible (˜1 mm in diameter), ASO-ctl-RBCEVs or ASO3.2-RBCEVs was injected into intratumorally every 4 days. Naked (unloaded) ASO-ctl or ASO3.2 was also included in this experiment, in order to examine whether RBCEV-based delivery improves the uptake of ASO into tumor cells. As expected, intratumoral injection of ASO3.2-RBCEVs significantly inhibited tumor growth (FIG. 6D); while there was no obvious difference observed in tumor growth between mice treated with naked ASO-ctl and ASO3.2 (FIG. 6E). Altogether, ASO3.2 could effectively inhibit tumor incidence and growth in vivo.

ASO3.2 Specifically Inhibits Cell Viability of Cells Derived from HCC PDXs

To evaluate whether ASO3.2 could be a promising RNA therapeutics for cancer treatment, the effect of ASO3.2 in HCC PDX-derived cells was examined. As HCC is a highly heterogeneous cancer, cells derived from PDXs that were generated from different regions of the same primary HCC tumor (e.g. PDX22-T1 and PDX22-T2) were also included in this study, in order to investigate whether ASO3.2 specifically targets tumor cell populations expressing edited AZIN1. The editing level of AZIN1 was first examined in all PDX cells. Four PDX cells (PDX-1; and PDX-22-T1, T4 and T5 which are from different sectors in PDX-22) have more than 20% of edited AZIN1 transcripts (FIG. 6F). As seen in FIG. 6G, PDX1, an AZIN1 editing-positive PDX line, demonstrated significantly reduced cell viability upon ASO3.2 treatment; while no obvious changes in cell viability was observed in a non-AZIN1 editing-positive PDX line PDX22-T3. These data suggested ASO3.2 has a specific inhibitory effect on cell viability ex vivo.

### DISCUSSION

Dysregulated A-to-I RNA editing is implicated in multiple diseases in human including cancer. Dysregulated A-to-I editing is a key driver in the pathogenesis of various cancers, such as breast cancer, glioma, multiple myeloma (MM), chronic myeloid leukemia, HCC, CRC, gastric cancer, and ESCC. Transcripts aberrantly edited by ADARs in cancer tissues such as AZIN1, Gli1 (glioma-associated oncogene 1), and DHFR (dihydrofolate reductase) remarkably contribute to cancer progression and metastasis. Unlike DNA editing, genetic information manipulated by RNA editing is reversible and tunable. Since ADAR1 has multiple functions that are critical for normal development such as hematopoiesis and organ development, simply modulating the expression of ADARs may cause considerable off-target effects. An alternative strategy is to disrupt ADAR enzymes to specific editing sites at target transcripts.

In the present study, it was uncovered that the 3′ end sequence of exon 12 of AZIN1 forms dsRNA with the editing region. Thus, multiple 2′-O-Me/PS-modified ASOs and PNAs that target either the editing region or the ECS were designed and synthesized. It was observed that 1) ASO1 or ASO3 could either substantially inhibit or completely abolish AZIN1 editing in vitro respectively, 2) ASO2, 4 and 6 were incapable of binding to AZIN1 duplex, and 3) although ASO7 could bind to AZIN1, it still failed to inhibit editing in vitro. All these observations suggest that the 5′ of the editing site position and 3′ of the ECS may favour the binding of ASO to AZIN1 and inhibition of ADAR1 reaction, which may provide a useful model for future understanding of ADAR1 substrate binding and deamination.

Moreover, it has been reported that PNAs incorporating modified nucleobase such as thiopseudoisocytosine (L) and guanidine-modified 5-methyl cytosine (Q) can selectively bind to dsRNAs over ssRNAs and dsDNAs in a sequence-specific manner. Besides, PNAs have a neutral peptide-like backbone, are chemically stable and resistant to nucleases, and offer enhanced specificity of RNA sequence and structure recognition. In this study, the inhibitory effect of PNAs on AZIN1 editing was also tested. As ASO4 (20-mer) was unable to bind to AZIN1 duplex, it is not surprising that the antisense PNA ASP1 (12-mer) was also incapable of binding to AZIN1. Although dsRNA-binding PNAs DSP1 and DSP2 could bind to AZIN1 with modest affinity, they failed to inhibit editing at nanomolar concentrations, possibly attributing to the insufficient blockage of ADAR1-AZIN1 dsRNA interaction by DSP1 (10-mer) or DSP2 (8-mer) due to their rather short length. All these observations imply that a relatively long and chemically stable ASO (e.g., 2′-O-Me sugar ring modification in combination with PS backbone modification) may be an optimal chemistry for RNA editing inhibition.

Although ASO1 demonstrates very promising editing inhibitory effect in vitro, it was found that ASO1 and other ASOs that target the 42-nt editing region led to a substantial exon 11 skipping, probably due to the blockage of splicing regulators (e.g SRSF1, SRSF3, SRSF6) to the editing region, suggesting that the editing region is not targetable. Moreover, even though the ECS targeting, 2′-O-Me-modified ASO3 could completely abolish AZIN1 editing in vitro, only ASO3.1 and ASO3.2, which have the same sequence as ASO3 with either complete or partial PS modification respectively, could effectively abolish AZIN1 editing in cancer cells. This may be attributable to the advantages of PS modification such as strong resistance to endo and exonuclease digestion, increased serum stability, reduced renal clearance. Notably, ASO3.2 demonstrated higher specificity to inhibit cancer cell viability via repressing AZIN1 editing than ASO3.1, possibly because of the detrimental consequences of non-specific binding to proteins and other nucleotide sequences that results from PS modifications. This finding was further confirmed by the observation that ASO3.2 inhibited cell viability of AZIN1^{S367G}-expressing cancer cells and cells derived from HCC PDXs, but not AZIN1^{S367G}-null cancer cells, PDX lines and normal hepatocytes. Further, in the pre-treated xenograft tumor model, ASO3.2 remarkably inhibited tumor incidence and growth. This observation was also supported by the intratumoral injection model in which ASO3.2 was delivered into tumor cells using RBCEVs-based delivery approach, which shows that intratumoral injection of ASO3.2 that were loaded into RBCEVs, but not naked (unloaded) ASO3.2, significantly suppressed tumor growth.

Altogether, the data indicate ASO-mediated inhibition of AZIN1 editing effectively suppresses tumor incidence and growth, which underpins the possibility that a large number of cancer patients, particular patients with HCC who demonstrate high editing level of AZIN1, may benefit from an AZIN1-targeting, ASO-based therapeutic strategy. Owing to the liver architecture and rapid endocytosis, hepatocytes are highly receptive to ASO uptake. Conjugation of a tris Ngalactosamine (GalNAc) targeting domain to ASOs led to a 10-fold increase in the activity of ASOs, significantly improving the second-generation ASO chemistries and increasing the potential of ASO therapeutics for treating liver diseases including HCC. The discovery of this ASO-based RNA editing inhibitor in the present study provides an attractive approach for targeting cancer-associated RNA editing substrates.

1. An oligonucleotide targeting the core editing complementary sequence (ECS) of AZIN1 gene, wherein the core ECS of AZIN1 gene comprises the sequence 5′-GCTTTTCC-3′, and wherein the oligonucleotide comprises one or more nucleotides with sugar modification and one or more modified internucleotide linkages.

2. The oligonucleotide of claim 1, wherein the oligonucleotide is a ribonucleic acid (RNA) oligonucleotide.

3. The oligonucleotide of claim 1, wherein the oligonucleotide is an antisense oligonucleotide, or wherein the oligonucleotide is an antisense oligonucleotide which is a non-degrading antisense oligonucleotide.

4. (canceled)

5. The oligonucleotide of claim 1, wherein the oligonucleotide comprises a sequence complementary or partially complementary to 5′-GCTTTTCC-3′, or wherein the sequence complementary or partially complementary to 5′-GCTTTTCC-3′ is located near the 3′ end of the oligonucleotide.

6. (canceled)

7. The oligonucleotide of claim 1, wherein the nucleotide with sugar modification is selected from the group consisting of 2′-O-methyl modified nucleotide, 2′-O-methoxyethyl modified nucleotide, 2′-fluoro modified nucleotide, 2′,4′-bridged nucleic acid modified nucleotide, locked nucleic acid (LNA) modified nucleotide, and morpholine ring modified nucleotide.

8. The oligonucleotide of claim 1, wherein the modified internucleotide linkage is any one of phosphorothioate, phosphoramidate, or phosphorodiamidate; or wherein the oligonucleotide comprises phosphorodiamidate morpholino oligomers (PMOs).

9. (canceled)

10. The oligonucleotide of claim 5, wherein at least five nucleotides in the sequence complementary or partially complementary to 5′-GCTTTTCC-3′ are modified with sugar modification.

11. The oligonucleotide of claim 5, wherein the oligonucleotide comprises at least three modified internucleotide linkages in the sequence complementary or partially complementary to 5′-GCTTTTCC-3′, or wherein the oligonucleotide does not comprise any modified internucleotide linkages in the sequence complementary or partially complementary to 5′-GCTTTTCC-3′.

12. (canceled)

13. The oligonucleotide of claim 1, wherein at least 50% of the nucleotides are modified with sugar modification.

14. The oligonucleotide of claim 1, wherein at least 10% of the internucleotide linkages in the oligonucleotide are modified internucleotide linkages.

15. The oligonucleotide of claim 1, wherein each nucleotide in the oligonucleotide is modified with sugar modification.

16. The oligonucleotide of claim 1, wherein each nucleotide in the oligonucleotide is modified with sugar modification and is connected to an adjacent nucleotide via a modified internucleotide linkage.

17. The oligonucleotide of claim 1, wherein the oligonucleotide is of at least 70% identity to the sequence 5′-UUAAGCUUCAGCGGAAAAGC-3′ (SEQ ID NO: 5).

18. The oligonucleotide of claim 1, wherein the oligonucleotide comprises or consists of the sequence 5′-UUAAGCUUCAGCGGAAAAGC-3′ (SEQ ID NO: 5).

19. The oligonucleotide of claim 1, wherein the oligonucleotide is of at least 70% identity to the sequence 5′-mUmUmAmAmGmCmUmUmCmAmGmCmGmGmAmAmAmAmGmC-3′ (SEQ ID NO: 6), 5′-mU*mU*mA*mA*mG*mC*mU*mU*mC*mA*mG*mC*mG*mG*mA*mA*mA*mA*mG*mC-3′ (SEQ ID NO: 7), 5′-mU*mU*mA*mA*mG*mCmUmUmCmAmGmCmGmGmAmAmAmAmGmC-3′ (SEQ ID NO: 8), or 5′-mUmUmAmAmGmCmUmUmCmAmGmCmGmGmA*mA*mA*mA*mG*mC-3′ (SEQ ID NO: 9), wherein m represents 2′-O-Me sugar modification, and * represents phosphorothioate linkage.

20. The oligonucleotide of claim 1, wherein the oligonucleotide comprises or consists of a sequence selected from the group consisting of 5′-mUmUmAmAmGmCmUmUmCmAmGmCmGmGmAmAmAmAmGmC-3′ (SEQ ID NO: 6), 5′-mU*mU*mA*mA*mG*mC*mU*mU*mC*mA*mG*mC*mG*mG*mA*mA*mA*mA*mG*mC-3′ (SEQ ID NO: 7), 5′-mU*mU*mA*mA*mG*mCmUmUmCmAmGmCmGmGmAmAmAmAmGmC-3′ (SEQ ID NO: 8), and 5′-mUmUmAmAmGmCmUmUmCmAmGmCmGmGmA*mA*mA*mA*mG*mC-3′ (SEQ ID NO: 9), wherein m represents 2′-O-Me sugar modification, and * represents phosphorothioate linkage.

21. A pharmaceutical composition comprising the oligonucleotide of claim 1.

22. (canceled)

23. (canceled)

24. A method of treating cancer in a subject in need thereof, comprising administering to the subject a therapeutically effective amount of the oligonucleotide of claim 1, wherein the cancer is associated with AZIN1 pre-mRNA editing.

25. The method of claim 24, wherein the cancer is any one of liver cancer, hepatocellular carcinoma (HCC), esophageal squamous cell carcinoma (ESCC), non-small cell lung cancer (NSCLC), and colorectal cancer (CRC).

26. (canceled)

27. (canceled)

28. The method of claim 24, wherein the level of edited AZIN1 pre-mRNA is at least 15% higher as compared to the level of edited AZIN1 in a sample obtained from a healthy subject.


SEQUENCE LISTING (52 sequences)

SEQ ID NO: 1 - DNA, 29 bp, Artificial Sequence
  Synthetic sequence
aagaagacagcuuuuccgcugaagcuuaa

SEQ ID NO: 2 - DNA, 42 bp, Artificial Sequence
  Synthetic sequence
ugagcuugaucaaauuguggaaagcugucuucuuccugagcu

SEQ ID NO: 3 - DNA, 37899 bp, Artificial Sequence
  Synthetic sequence
ctaatataaatactggcgtcgctggcgccgccttctcacactttcaggctctgatcgcggccgcagtttttccttttttcttctgccgtcgccttctctgcctcttctcatcctttctcgctctgctgctctgcagtgtgacgagtccgaatcctcttcccacccagcccgcgcctttcttcttttgcctgcgctgttctatttctccttcggccgccgccgccactgctgcacacagctggtgtcggtgccgcgcttttacccccaagtcgttcccgcagcctatggcccaggccgccttgggtatttctgctcaaggtaaccacatccctctttaaaaattccgccgaaaaagagaagacgctttacccgactctttgggccgttatctcacgtgagtaccgagccgaggagcgcgggaggggccaggacgcggggctggccttggggggcggctggggggctccggctgggccccgccgcccttaagacgtccccgcagcgccccgcggccggccgcctcgaacatggcggcggcggctgtctgtgcccccggcggcggcggcggcggcggggctgggcggcgggccgcgcctcaggaccttcgggcggctgctggacgcttggcggagccggagccgcagcagttggggcgagggggcgtgtgccagtgggggagcgcgcgggggtctcgccgccgggggtcgccgtcccctcctggcggcgtgcggcccccacccctctcccgtggtgcattgctgtttccggggagggggcccggccgcagccctgaggggccggggtcccgcagcccctactccgcggtgcgccgccgccacctttgcctcccgaagcccggcgggcgggcggcgctgtgggctgcaacggctggggtcctggttatgaatggagcggggcgggccccgcgccggcctctggtgacagctgcggaggaagtcggattcctggggggcgcggggagcgcggtccccctctggtggaggaaagttgacgccacttggggcctggcggcggggaagccccggtgtcaggtagccagcgagaggcttttggccccttctggaagcctcgaggctgctgctccgcccctgcggcgctcctgattgggtcaagtcgccttcactcaccccagtcacctcctggagcctgaggattagtcaccatggcttcgtttttccgtccgcgccatggttcctaggcagaaagagggagattggtagacgggtggcttatcaactactgtcttgactgccaacagttggtggtggtaacatgttctaacccttacacggtatcctgaatgtgtgtttcgtagacaaagctgctggttcagaaacccgaaacagtcggggacacagtttaccgcctcatgtttcagcacatttaactgaattttgtggtggcatcttggttatattagttctgtgtcccgtcgtgtttctatcgtgaattcccagactgggtgggaatctaaggactcaaaggagggggaattgccttgtgctcagcgaaggttgatgtagaggtgcatcaaagcacaaagtagaagtacatcaaagcacatcaaatacagcaagggttggcagaaacatggctcatgattgtctagaaaggccctcataattcattaatttcttttacatatacgagagcgtgcagatgctgatcatgattagaaataagccccaaggagtttaggaaagactccaaaagaaaatgattatcggtggtgataagtgaggtcgagtagtcagtgcctttctcccccaaggttttatcacatacgttttcaaacagaaggtgaaaaagagttatacttccacctagattctgtaattaacattttaccttatttgtcgatgcatttcaaagtaaagttggagacccctccaactttagtatgtgcatattaaatagagctcagtaccgcttactattttttaaatcttagagggtaaaattcacatcagtataaacccacaagtcttaagtatgcaatttgatgaacgttgacaaaaatacagctgtgtaacctaaaccttttgtcaagatgtagaacatcgccatcaccctcaaaagttttttcttgtcctttcaaatcgtcttccagtcaccacagagccaaccacgatcttggttttttttttttggagtctagctctgtcgcccaggctggagtgcagtggcacaatctcggcttactgcaacctccgcttcccaggttcaagcgattctccttcctcagcctcccgagcagctgggattacaggcgcccaccacgcccagctaatttttgtatttttagtagaaaaggggtttcactgttggtcaggctggtctggaactcctgacctcatgatccgcccatctcggcctcccagagtgctgggattacaagcgtgagccactgcgcccggctgatcttgatttttttttttaaacccatagattattttcgtctattccagactttccacatgaatggaatcatacagtatgcattctttggtataaaaggcttcttttaactcagcacatttttaatattcatccctgttgtctttatctgtagattatcagtagattatttgtgttgctgagtagtagcccattaggtgactgtcccttggttgttttttttaaaaataaactttattaggccgggcgcggtggctcaagcctgtaatcccagcactttgggaggctgaggcgggtggattgcctgaggtcaggagaccagcctggctaacatgctgaaactccatctctactaaaaatacaaaaattagccgggcgtggtggctggctcctgtaatcccagctcctcgggaggctgagtcaggagaatcgcttgaactggggaggcagaggttgcagtgagccgagatctcaccagggcactccagcctgggggacaagagcagcgggacttcatctcaaaaaataataataataatgaaaaataaactttattttggagtatcttagttgcgcagcaaagtcacaaagatacagtttcttcatacttcccacccagttattagtaacatcttacgttattacagtacatttgttaaaactaagaaactgactttggcactatgaaacgttcgagacttttatttggatttcgccaatttttctttaatgtctttttctgttccagaatccaagttaccacattgtatttagttgtcatgtcttcccagttttctttggtttgtgacagtttctcagtcttcctttgtttttcattaccatcacagtctagaaaagtgctgccagatatctggtagaatactcccctcccctaaacttggggtttacctgatgtttttctcatgattagactggggtttggagtttttacaaagaataccacagaacgattttaaaaatcattggtaaaggcattgtgttttcagagacgtactgataattttgagatactaagtttgtaatagttactgtcaaagtgatagaaagtttttgtagttgggcacggtggtttgcacctgtaatcccagcacttcggaaggctgatgcaggcggatcacttgagctcaggaattcaaggccagcttgaattcctattttatttttaagaaaataaaataggccaggcgtggtggctcatgcctgtaatcccagcactttgggaggtcgaggtgggtggatcgcaaggtcaggagttcgagaccagcctggccaatatgatgaaaccccatctctactaaaaatacaaaaaaattagccgggcatggtggcacatgcctgtaatccgagctactcggaaggctgaggcaggagaattgcttgaatctgagaggcagaggttgcagtgagccaagatcgcgccactgcactccagcctgggtgacagagcgagactccgtctcaaaaaaataaaataaaataaattacataaaatgcttcttgtttcttactgaccaaccgagccctctctgtgctattctctaaggtgttagagaatttgcagtgtttacatacaccattctaagtgaaaataatttatttttttccatactgccttttctccgcatttccatttaacaagtccttcttcatgcttttttatcctaactgtccggataaacattctatctatcctgtatgtaagtgcttataagaagtgcttatctatcctgtatgtgtttataagaagtgcttatcctatgcaccttttgggtcttgctgtgttgcccaggctggactcaaattcctgggctcaagcagtcctcctgccttaacctcctcagtagttgagactataggtgcatactgtcacatctggctcattttttcttttcttttctttttttggagacaggttctccctctgttgcctgggctggagtacagtggcacaatctcggctcactgcaacctccgcctcttgagttcaagcaattcttgtgcctcaggctcctgagtagctgggattacaggtgtgcaccgccatgcccagctgatttttgtatttttagtagaagatgggatttcactgtgttgaccagactgatcttgaactcctggcctcaagtgatccactggctcagagccactgcgcctagccctattttttgtatttaaataatgcctttcaaattctagagccttcattccctactagtatctttttgttaaatggaaatgttgtagggttctcactgaggaacattctggaatgtctgacgtttaatgtccataatgtttcacatcattgctcttgggttgcattaagcagcaaaatagctgtggatatatgtattgagaaaaagatgaataatgtcttacctagggtatctgatcctattccttaggataattgagaatgatctgtccccagcatgactcaactttttatttatttagaaaattacgttggtgttgaaagaatcagaatgttatggagtagtacattatttggaatctaatgtaagattattatcatcattgcatgtttgtaggagttctttcctcatcatccataaatgtcattcattgagcatgtgatcagcttctcctagatagatgaaacattttgcctccaccaggagtgccttgatcctggtgagatacactttaggtatgcctacagtgcttctgaaatccctccaactcttgactttataatttctggttttctggcttccttcttaccacccctttctagcaaacactgtcagattattttacttgaaaacagtaaccactgaattatacatagaaacataggtatatcattttagcttagaagtgatgagtggtaacaatgtctaaaactcacctaccatgttgtatagtgtgctgtgcacttatcacatttatttgttcctggtgataatagaatgaattatgtccaaataggttagatatgagaagataaatcttaagagattagttgcttaatattataaggaagttctcaagttattttatgtttttcaaaagttatgttagatacatatataatgcattagctaagtcataaaatcttttggtattgtggtggatttttacatttaaaatgtaaaagacaaatatttgaggagtaaaagagtgttctgggtggttccaaactattgggtcatgctagtacattattcatataaagtcttgtatatacactaggttggtgagggtacttggaattttttaaattctggaccacttaaaaatgtggaattttagaggacaaataaaagcaaaaatgaagttttcatcttcaaaaatgaaggctggtaactcttgagtaggcagataaaacgacttggtaagatggctatgacatcaagttctgtgaattttttatagcagcttggaaatgtttactgcataaagtacttggtactgtggtaaaccctttgaaaatatcacttgtgtaatgtgactttaatgaaacattgttataaaatgaacacgtattctacagttcatgtttaagtcagcctaaacatttttttaacataggactatcttctgctacttttatttccttcttttagggcgaactttctgaccaagtatacaactacccagagggcctaggagaagtgctgtatagagagcagttcgacttcaacgctgagccaccttgggaacctagctgatgataggggggttccatctcccaacttgtccatgtaagtatttgcacatttgttggtggggaggaggtctttaaaatcgaaggaagagtgctgccttgcttagtatttttcagaatttgaaattgcatttacctcttaaagtgcagtgacttttaaatttctgaaatagagataagacagttactggttacagacttgactgatttagttgtgtgcattgtttaccaatgactggatcatttaaaactatcttgtatgcataacttagaaaatatacgttgaagagggcttttgggaaataaggtatagtttatcacatcattcaaccatttaacttagtgaaatgatttttgctactcagctggtaggaacaactaactgattgtttttagaggacttgctgaaagatgatagagatagatagagatacatatatatatctcaatttatatatgtatctaaatatgtatatggtattaaaatatatgtatggtattaaaatatagatatttaacagattttcaaggatagtttttgactatacttggttttctatgtgtgaattttactttagaattgagcccctgtgcaaaatgtaattccacagtaaactgaatttgggctactagatttgtgtgggttcatttaaaaattttgtggtatatagacttattgttacggaaattcagattttgttattaaaatttaagtgtttttaagtttggtcagagacttagaggtaagttttcaggaaagctgagggtatcagttttagtgtttctttaatctgatcttgttaaacagatccctattcgataaaagtgtttgctctaagcaaacatttgatgttaatacaaattagaattttagactcaggtttttaaggctaaagagaaggaaacacagtttaaatagtaagatttcttgaaacctttgacttagaatgttggctagagggtttcctatactggctttctcctacctgtgtaaatttgagcaaatgtgaatcccagctataacataatgttggactggaacagttctttttgtgttaaactggcataatttctttttttgagtgtattactgtccttggttatgcagtgtagtcttcggacgaattgcattagcatcctcacctagaacagaaatacagaatctcagcctcacccaaacctgaatcagagtctatatccaggtggcttgtgtgcacattaaagagaagcagggtcattgctgtttcaatactaagtcagttgcctaatttttgttagtccatctgttctatttgcaacatacctgatgtttttctcccaagttgacttggagatataaaacctttttgggtggtgcaatctgttaaagttaacatcatagttgtgaatccattctaaggcagaaatttaagaacaagcttcgtcttttgaggaagaccgtaataaaagataatccttcttttaattcgtgtctcttgctttaaagaaaagttaccttgcattatagttaatatttctactggtgtttcctcttagaaacttgagagactattgtcagtgagctgaatgattctgcaaaatcagggaaaagtagtctttagattaataatgtaggattcagaaagtctatagtgtctccctctttgtgggtaggagggaatgggagcagatagaagctaaagataatttattctgcaggttctggcagtcagaataactggcttttaactttattatgcccgggtacaatgcatattatgtcaaacaaatataacttctttaaatattcacatgtaatcttctcattttgcatgtctagtatttgatgctttgctataaagctacaggtccacaatccctttttcatagaattctgaaatccaaaaagtctttaaaaaaatttttttatagcttccttgatgtgaccttaactgatttgggaaaaaaaaaaaaaaacaaaaaaaacttgacctggatctgtgctgatttgaggctatttttgtctttacttatcccatttagtgttaatattcctaaattttgcttcagaaatattgtggttgcagggtgctgactcattctcctgaagaatattacataacatatgtaaagtatgtgtactacattatggttcaaaatctggagaattctgaattcacaaagatacctagtcccaaaggtttcttgactagaatttgtaaaattggcaagagaaaattccgcattccagaaatgtgttatccttacaaatcaataagagtttgctctattggacaccgaaatcctggggttaattttctgtagatcaacaatattatttgaaaaccattaaaaggagttaaaacagcttcatggaaattatttggcccatagcattgcaaagcatgtagctatatgaactctgcttgaactggtccactaggattcccgtgcaggtagtctgagatccagaattggcttcagagttaggttttaaatcaaccacctgaattttgatggaataaactaagtatctggaaattttggaagttgcttacataatccttttctttcataatttagaatgtgtgtatgatgtagtacgcaaacactgtaaaaatgtcttaacaaaatattttaaggtcaatatcaaagattaatgggcccctaattgtgaaaaagtttttgtgagccttttgagaaatcccctaggccaggtgaggtggctcacacctgtaatcccagcactttgggaggccaaggcgggcggattacatgaggtcaggagttcaagaccagcttggccaacatggtgaaaccctgtctctactaaaaaaaaatataaaattagtcgagcgtcgtggcaggtgcctgtaatgccaactactcgtgggaggctgaggcaggagaatcacttgaatctgggaggcggaggctgcagtgagccgagatcgtgccattgcacttcagcctaggctacaacagcaaatctccgtctcaaaaaaaaagaaatcccctaggtcttgatgctggagtaaaggttttccagtgggctagttttctatttattttggttgtaggtttaattataattaatgaattagcctaatggaaaatattttaaaacttaaaaagctaagtttgcttctcatagtggatccagtgatgtggtcagattcattatcacataccctttaatgttgtgcagtatggaaaagggattcagttaattgatttgcttaaatgggctcttaacaagtttgctttaaaaagtttttttttttacctggttgtcattagtcggctctatatattcatgtaggaacttcagaagaacatcagcattttaaatattcacattttgtctcctaatctttagtatttacacatgccttagtaggagctccctacctaattaggattattttaactagggaaaaaaaatatatatatatatatatattttttttttttttgacagagtctcctcctttcgcccaggctggagtgcagtggcacaatcttggctcactgcaacctccgcctccctggttcaactgattcttccgcctcagcctcccaagtagttgggattacaggcacccaccattatgcccggctaaattttgtatttttgtggagatgggtttcaccatgtggccaggctggtcttgaactccagacctcaggtgatccgccccccttgcctcccaaaatgctgggattataggcgtgagccaccgcgcctggctggaaagatgttctttacgaatttgctagtactccttcctcatgtttcactgtaagttatcagtaggaaagggaggttgtccgctgggtgcggtgcctcacacctataatcccagcactttgggaggctgaggccggcagatcatctgaagtcaggagtttgagaccagcctggccaatatggtgaaaccttgtctctactaaaaatacaaaaataaattagttgggcatggtggtgcacgcatgtaatcccagctactagggaggctgaggcctgcttgaacccgggaggcggaggttgcagtgagccaagatggtgccaccgcactgtagactaggagacagtgcaagactgtctcaagggaaagggaggttgttgaccctgactgcagagaaaggggaaaatttcagttttgatatttggtacttttttttcttttctccccccaccccccgatttaatttctcacagatgatatttggtactttaaggatactgtgatatgagcaaatgtaacagaacattcattaatggcggttgatactctttgaccaaaaagatgagtaaaaccactatcgttgaaatacacagtctgtatatccccttggcatacataaagtagagtcaatggtgtgtcagtgcttgtgtaaagtagcagaaaaatattctgtaaaagcacaatgtgaatactagcatattaatggataaacaaggttttagagacatggtatttcttgctttgagcaaatttagcattttttttaggaaggtcaaaaagattggaaagcaacgtagaccagttttttctttcttttttgagacgaagtttcgctcttgttgcccaggctagagtgcagtggcgcgatttcagctcactgcaaactccacctcccgggttccatcaattctcctgcctcagcgtcctgagtggctggtattataggcatgcaccaccatgcccgactaattttgtatttctagtagagacagggtttcaccatattggtcagacttgtctcgaactcctgaccccatgatccgccttccttggcctcccaaagttctaggattacaggcatgcgccactgcgcccgaccatagaccagtttttaaacgggataaatggcttttgcatttattacagaggttagtttagctgagaaaattaatactgactaccaatctgtgtgttccatcacagtcttcacccaacctgatcttgtttcagagttttccagtttgggtctccccataccccaccccaggtaaaccaatcccttttgcgatatttaacttccattagaataaatgagatattgcatctatgaaaaaggcttgtaaaccttaaaatctagtatatggcaggctactgttctaaactggccattgttcagataagcctaaggagactcttccaagttgaagtgcttacatactggtgaggccccagggagcacagatattttgtccttcctccattccagggacatccatgcccagtcaaatcaagcaatttgtcacaagttaactaaagcagatatttagctctccttttcgttaatccactgttttttacattatgcatgagtagaaaatactgtgtagcatactgggttacggggattaggatagtcaggttcaacctgcccaggccatagccagccatcatgaggtccagccacgttgtgcatgccaccacgccctgctaatttttgtattaaaaaattttttttttactattatttttttgagatggagtttcacctttgttgcccaggctggagtgcagtggttcagtctcactgcaacttctgcctccttagttcaagcaattctcctgcctcagcctcccaagtagctggaattacaggcacacaccaccatgcccagctaattttgtatttttagtagagacaaggtttcgccatgttggtcaggctggtctcgaactcctgacctcaggtgatctgccctcctcagcctcccaaagtgctgggattacaggcgtgagccaccatgcttggcagtatgagctttatgttgacttcagattactttttatctgcttcttgctctttactgcaaggatcctctgatgcccagtttgcaaagatctcctaaacaagtatggtatcttcttagctcatgtaggttcagcatgaacatgttatatttggcatgaccaaaggtaggatggggttatcctggtatgttcaacatggaaattctgacaaaacaatcaccccaaaaataatactcctaatgagaggtttgctatcctaaattcatgaagagctccttcagataaataaaaggcaaaaacaagaaactgagcaaaagacttgaaaactgtttgccactaacaatctagcagttccagtcccaggtatagactgtagtccctagatcagtgctacttaaactgccagccagtggaataaggactctagtactagaatgtaaagtaccacattgcccaacactgttttagggtcacatagaagaataaaaccattaagtaacaaaggaatgatgattagagaaggtctaaacaaattctaagggagaggtacaggggcctctgagatattacatcttatttctttctttttttttgttttctgagacagtcttgctctgtcacccagggcagtggcgtgatcttggcccactgcaacctccacctcccaggttcaagcgattctcctgcctcagcctcccaagtagctgggattacaggtgcccaccaccacgcccagctaatttttgtatttttagtagagacagggtttcaccatcttggccaggctggtctggaactcctgatctcatgatccacctgcgtcagcctcccaaagtgctgggattacaggcgtgagccaccatgtccggccagcatctgatttcttaataacctgcttaacttggtcttggttgcaattactgtattgcacatgtattctatatgttttccttgggtgagtagtggacaggcaggcaatgagctctgccttaaggtaaccaaaatagtgtgtctctttaaaggattattccctatgcttaattgtaatcaggaaagctagaaaacttgaaattaatatgattgtgtacttcatgttgtattgtaatagttatctttaatgttatcacatcttaattgccaaaagggatataaatatttcacttgagtgcaggtacacacacactttagtacacacacattttctccagaaaaaaaatttagtcttcacattgaatagtcaagacatttttaagggagtctaattgtgttaattccacgtgcaaaaataatattgctgtgagaattgtgtattttgttttctcattaagctacattttaatatttatggtgaggcagttcattgggcagtctttacacaggcacttcaaacttagcctgttgggcttataacttttgggattttttgcccccttatatatttcctattcattcttgttcttctcttttaaaataaatctttagcagtcacctttagtcttcttcctctaaggttactcagttttttcaacctcatcacctgaaaaataagtttaggggtatgcattgtgattactggctttcttattgaagcaagccttgtcctgatttatcctgagtactcaatgtaggctgttgttattgtctatcacttattttggggtccagggtggcagaagtttacctgatcttatgatcactaaatttataggatttcctaccagtttcagtatatagcttcctggaattcttttgtagtgtttcagacctactcaaggtctcacatctatggtagatgtcattctttagaagcttctagtgctcttctggtcaattacttttaaaggtattctatgtggtaagtaacattttctgttgtaggtagaatgcagaggggacttaagcaacgttgtatatttttaaagctgtgagctgggtgctaagtcatcaaatgcctatgtaaaaataaaagtagaaatgcacctaggcatcttcagagtaacagtaatagtgtaaatttcagaatatcttttctcatttttaatgagtaaaaatccttttagaaaaatggttgatgtattaaggttgggtttcctgcttaggtatctctgaaacagtattctcaacctttcagctctccctgttctacttccagggaggtcttcacttcagaaatccaagactcatattcatccagcttggtgtcaagtgggctgttgctgccagaattatcttgtgattatttgagagatgtatcagtttcttctgaagtacaatcaactgtagaagcctttgtagcaggtgtgtaacaaggcaagtgcccagctcctgcagccactactcttggagctaccctgtcgatgaacatgaggactcagcggcctcatgaaaggctccagggagcagtgtgtggttctttggagccttttgtttcttttggcagctcttcatggaaaaatgtattaaaaaaacacccatatgaaggtgtgaataagggatgttacatattttttcttctcagttatgagtctaagaggaaattattaataaaagtagtattgtggttatacgctactgtattagtgtaaaataacctgagctttctcaaccgtatgttaataaggtggttgaaaatagcttaaaagtgcttgtgagtagagaatactttctagtgtctattgataatactagacatcagagctagatttgctgttcagttctaatgaagagaatctaagatttacatggagtagtttttttttcaagtttaatataattagttcaaggtccaatagcaccagaatgtgttgcagccagtaaatcagtcctgtttctgatctgtaagccagaagaagtggtacagtttcttttgggaattatttccactcagaattgcaaggagatacttgatatatcaccttggtttgagtctgcattcttagtggagggtatggagacgtcctggaaagagtccctgtgcgggggtttttgtttcttgtttttgttttgagatggagtcttgctctgtcaccaggctggagtgctgtggcacaatctcagctcactgtaacctccgcctcctgggttcaagtgattctcctgcctcatcctcctgagtacctgggactacaggtgtgcaccaccacacccagctaatttttatatttttagtagagacaagtttcaccatgttggccaggatggtctcgatctcaacctcgtgatctgcctgcctcggcctcccagagtgctgggattacactgtgcccagccccctgtggtgttgaagattgaggtagattaacttagtgaatctggagtaaggtataaactgctcacccccgaaacatgcacacttattttcatgcttgctccctagaacatgctgtgtcttaagacttttggtttgacatattgtcgccaattaactttaatacctcctgaaaacgttttggcaaaatatttcttagtgctttcagtaaggcataaatgtcacaacataaacctttcttttttttaacaatggaactgtaaactatttttgctaatatattaagaaaactgcaagcagtgatcccagaggtatgatttgagctgcttttttaggactctttcagtgagattggggcacaacatatgaaagactcaagcaacctacctcacccacctacttgtcccttttcagttggaaatactgggtttccaagtggcatttcatttgaacaaagagtttgttgcctcaagatatattagaaaatttgaccaggcatagtggctcacactggtaatatcagcacttaagagaggcagagacaggaggatagcttgagcccaagaattagagactagcctggccaatatagtgagaccccattctccataaagagaaaaacaaaaaaagaaaacttgttgctgcagatgtttgtgttttacagattcttttgaggaattggcttttttttttttttttttttttggccttttagtctttaacagtagctgatagttgagctttgcattcacaggcatgggtagcttaatgacagagatagattctgagaaatgtgtcgttaggtgacattgtggttgtttgaacatcatagtgtacatacagaaacctagatagtacagcctacgacacacccaagctgtatggtgtagcttactgctcctaggctaccatcctgtataccgagttactgtactgaatactgttgacagttgtaacacagtggtaagtatctgtgtatctaaacatagaaaaggtacagtaaaaatatggtattatgagaccaccttcatatatttgatctgtccttattaaaaatgttgttatgcaacgcatgactatacatctggattgcttgcatatcaaagttacgggcttcttttaagaatgtgaaagagctggacagagtggctcatgccggtaattgcaacactttgggaggccatggtgggaggattgcttgaggccaggagcttgagaccagcctggggcaacatagtgagacactatgaacaaagagttttctacaaaaaataaaaaattagctggacatgatggcatgtgcctgtagtctcagctacagggactgactgagtggaggtgggactgaaggctgtggtgggaggatcgcctgagtggttgaggcttcggtgggctgtgatgccactgcactccagtctgggtgacaagagtgagaccttttctcaaaaaaaaaaaaaatgggaaaaattgagctactttttcaatacacttccagggagttcattaccaccacccctaagtttaagaactccccactgtttgttgtgaagtcttttgtccatttgtttctgagtacctattctgggctaggaatttacagcaatctcaaagccttcaagagagcatcaaagaaaataaacctccattagagctttattgtttttttttttttttttttaagagatgggttctccctatattgctcaggctgatctcaaacttctggcctcacaaaatcttgcctcagcttcccaaagtattgggttcaatcaactactttattgtagttctctgtataatgaaagaaaacaaatgttaatgtactacatctgtggattcaggtgatccttaatccattttgtcaagtactgccaatatagcaaatgaattttatttcatttttgcaacagaatgattttgtcactcagctgggacgtggttaacaactctgctgttacttctgaagtaacagttccatttcaaagatgaatagtcaactccacaaacattttatctttcctttagaagaggtcatattttttcactatttttatttatattgataatgacactatatgtcagctgttttggtttagccttcagttgattaaaattatttagcttctataaaagtcttaatttatagtgacaagaagctggtcagtggttgcctggggtggagttggggatttgctgcaaaagggacatcatatgaagaaaggctgtgagtaggcgtgataatgtttttataccagtgtgaataaacttagcatttttcaacaagtatgagtgttcatatgtctgaaaggggttagttgattatgtggatatgataaagttcttttaaagagactgagaactgtgttggatttcccaaaaaaaaaaggagtcctgtgttttatagggatggggtgctggggaagcaaggttttcatattgttttaaatgtagtttgatcattagtatctctagagctaacctacttaattattctattcctcagtatatcaatggagataataccgtcattgtggttttaagggttaagagaattaatattgaatagatgaaacacttaggctgggagactgctataaacatgtttactgttagatttaagctagcttagcaggcagtcagagttgaacctatgaaattatatggtttgaaaatatttgggaaaaaatatctttatccatatcatcatactactgtagcagaagctctgaagaatgtaggtagtcagccagtaagacttgatccagtgggcctgatgtttttataagtactgaggatcctgactgttaaaggaaaattacccttcctaagaacactatggttgtaggaaatggaggtttatcttatcctgacttaaaagctagaccttctgtatttggcagcagttgtgtgttgtcgtttataatcaataaccaaaggttttcaactaagtactaaatactacatatatattagactatgcccagagacttagcaacagcaactgagatacaattaagtcctcattgaacatcatcagtaggttcttggaaactgcgactttaagcaaaaagacatagacagcaagccctcaaataacatttcattataatgttaagaaaaaaaatggtttcgttttacctcttacttaaaagacacagtttgcaagaagctattgacataaagtgaggacacaatttacttatttttttttccatttcaaagtattttatttaggccaaaagtcttgatttcgtattttttcatttattaatagtaagataaatgaaaactggctgctatgccaaaaaggttagtgatgttaaacttaacaggaagtttctaccttattttcttatatttatctttttaaatgagtgacctttatttttttaattgttcttagtgctcagttgaatatccccccaaaattggtgctttaaattgcttaaaataaacatgtattcttagcctaaaatgtctatattttctgggcacagatttacagcagtcctgttataacagtctttagagaatgtgcaggctgtgatttgacgtcactgcacaggtattaggtctatttagataggcctcaattaacttggtaaaataaaaacatcctaaacttttcagtctgtgctatatggcatgaaagatctgatgagttgaactgaatgtagttggcttcttggaaatgaagctgttagtgtttgaaaagttaaaactgagtatttgtatttcatgaaatttgactaatacagcttattaaggactgttttggtgcagccaaaatgctgacacttttaagaaccatcgaaatggagtatactttcaggacagtggccatcacccaagggtgaactctagtttggggcattagggagggtggaggggggtggtgggaggaatcaaggacacaggaaaggatgtgcaggttttaaaacaggctgcccagacagctggtcaatggccattattaatgtggcaaccagaaattagtaccagatattgagtagtatcctgtctggtggacctgttttatctcatttatgtcgaagttatttcctaaagtaacctaaggaccagtgaagttcaacaaagaagataaacctttatgtcagtatagtatgtgctgccttaggacaatgagtgctccgggggagtgggcaggtttatttcattgatttgagtggagagggataggatttagcctaaagtctttttaaaaatcagattacttagtaactggctcaggatgtacagatacctaatttacaaatgtagtttctaattgtgttatttttgcatttctggtaattgacaattttagctctattctagccagcactgaaacagatgtttttaatttatatgctctatgatttttttttttttttacaacaacctgttgaagaaataattaaggtagttaactgaggtttagagaaattgatgtagattctcctaaatgatatagccaggacgaggtcttctgactcctagtccagtgatttcaccaaagcaactcacctcataacattgctaacttgatacagctagtactattttagcttgaggtaaggcttaggcagtctgtatcaaagcttcagattgttccctcaagccaccctccactctcacccatttttattactttcacctatttgggaattttttttttttttttaggagagacggagtcttgctatgttgtccaggccggtctcaaactcctaggctcatgcaatctaccggcctgggcctctcaaagtgctgggattacaggcatgggccaccatgcccggccaggacttttatttataagacatttatctgcaattacctggcagtgctaatacgtggctgacagtatattggaagttgagactggtattttgatttcttggatgcagatacttatttcactagctttctttactcttaaaatgggtaaaattatgtgtgttataggaacgcaaaacaagataataatacagattgggtctgcaactctgtaagtaaggtgaccatataggcttgtaatacattgtgtatatatgtgagttaccccttcaaattatattgacctttttgggtaaatgtattttaattcatgtatttgcttttttaaatgggcgttaaaggaaattaataatctgatgtttctgaaattgtaaaattgcgtaaattcctcactaaattaggcacttagcacactttcattgactcgtacattatgtctattgaaataatacagacttttatataacttatattttgacagtttgttgcatattctaaggacccagacataggcttggtggcccgtctcttgtctttcctggtttatgactttcggctttgtggaatacggctgagatgaaaggatttattgatgatgcaaactactccgttggcctgttggatgaaggaacaaaccttggaaatgttattgataactatgtttatgaacataccctggtaagtgccatttctcaaatacagtgtttgtcattttccaagctctgaaacgagtgctttttaatttccaagatggtaatctgatcagaagtacttggttcatatgctatctgatttaacttgaccttcctcccccacacccactccccttgagtagtgatccatttttctttccaatccacattattttaaaatcctccctatggccctttaccttctatgccttttagcatctggcacccacatatgttctgtttattctgttgatgtgatgccagccaaccttctaatcagtacatggcttcttactcatttttctcaattttcttttttttttttttttttggagacgagtctcacgctgtcgcccaggctggagtgcagtggcgtgatcttggctcacgtgcaaccctcgcctcctgggctcaagcagttctctctcagcctcctgaattagctagaattacaggcgcacaccaccacacccggctaatttttgtattttagtagagacgaggtttcgccatgctggtcttgaactcctgacctcaggtgatctacccacctcagcctcctgaagtgctgggattacaggcacgagccaccgcgcccagcctcaattctttttttttgcttgcttgtttgagatggattcttacactgttgcccaggctggagttcagtggcacgatctcagctcactgtgacctctgcctcctgcgttcaagcaattgatttaggatccttatgcctcagcctcccgagtagctgggattacaggtctgtgccaccatgcccagctaagtggttttttttttgttttttgttttttgtttttttttttgagatggagtctccctctgtatcccaggctggagggcaatggcagtatctcggctcactgcaacctccgcctcccaggttcaagcgattctcctgcctcagcctcccaagtagctgggattacaggcgcccgccaccatgcccagctaattttttgtatttttagtagaggtggggtttcaccatgttggccaggctggtctcgaactcctgacctgaggtgatccgcccaccttggcctcccaaagggctgggattacaggcgtgagccactgcgcctggcctaagtttttgtatttttagtagatggggttttgccatgttggccaggctggtattgaacgcctgaccaacatgtgatctgcccgcctcagcctcccagagtgttggattaacaggcgtgagccactgtgcctggtctcagttgtattcttcagtggatattaatttactttcagataattttggttgtgtatgttcattgagaatttgataatttatcctgagtacttactctctgctgctctaggagtcaacttctacagaatcatatactcactgctacttaaggtgattttctttttttttttctctttgagacgaagtttcactcttgttgcccaggctggagcgcagtggtgcgatctcaactcactacaacctcggcctccctggttcaagcgattctcctgccttagcctcccaagtagctgggattacaggcgcccagcaccaggcccagctgatttttgtatttttagtagagacagggtttcaccgtgttgaccagtctggtctcaaactcctgacctcaggtgatccacccaccttggcctcccaaagtgctgggattacaggcgcgagccaccgcgcccagcctggtcttttttttttttttttttatatatatatatattagacttaactgatacaatctttctttattggaggaaatttcctgtattcaaccaaacttgttagataattgttttcatgttaccggtgttcagtaattgaatatccaaaattttctccttctcttgaccttaagtctgcagacgtgttcatcttcccctcatcctagcaaaaatccctttgtcttcctctgccattcaacttgttaaaacttgacatattggtcatgaaacttgtcatattggtcaaccaatttctgccaagccattcttcagttgccactgggaagctcctagtcaaattctgatgatcatttttatcacttaactttattcttctgactttttgcaacacttttcgggggtcagatataatctttgaaatattccttccatcccacatctaaattaacccacgtttagtagcattcaatgaggattactcctcaaccccattcttttctggctctggggtgacacttctgtgttctctaagtcattcagtccaccttttgtttgtcctctcttatttaaccttggtcttgccttcataacatcctggtattcatctcctccattcccactatctatccgtctttgcacatgtcattatagtgacctcctaacttatttcccttttcatcgtcctattgtttgcccaagcctgcttaattttgttgaatattcattaaatacaaggcttccctgctccctaaatttgtttttcgttgcctactgagaattttccagactcacatcatgcctaagaaaggaagctgaataccttgttgtttcatcccatttctcttgtggttcccgtaaataccttattttttcccaaattaaatataaactttgtgtctccggaataccatagatatcatatcttgggaggatggataacttaatacaactgttaaaattctgctgcacataatgtgcgttttgcatttatatcagttgggcactatattctaatgacattttcaaatactgtgaattaaagagttgaaaacagtaacattgtcttcatggattctcaaacagtatttggatgttgtacgtcctaaataagatttttttgcctgctatctaacactgagtaatatatgttctgtccaactgcataagtcatctctctagtcctagaatagccacaactttaaggaagattattcttattcaggcttatctgatatttggtagacttatgtacttttaaaatctaattttgacatctccatgtgcagactaacaaccttgtctttttaaaaatggtttcctggttggtcacggtggctcacacctgtaatcccagcactttgggaggcctaacatgggaggatagcttgagctccaggagttggaggctgtagtaagctatgattgcaccactgccctccagtctgttgctacagagcaagacctcaactcttgaaaaagtaataaaataagaggtttcccagtgaagatggtctcttattttaccatgttgtcttaaggtgcagttttaatggcgatatatagcttgttttatttagcatttctgattctacttactgtattaggaaattatatcctgaactgccaaagtattttaaacagtgggggaagcatgggagaaaataggttcccttccttcaactgccaaaaagtaaaatcaaggcagtttcattctgacttttgtcactgtaattgataatagtttttttatctcaaaggggagaatattttatatgctagtgtagcacagcaaaagtaatcaaatataagaagaaagaacaaatgtattcagagtctcttttaatgtttactaaatctagttaacaagctttaaatgtattactgtatgtaaatatatgtcttgacccatatatttgtggaggaaaagaggtgttctttcctgttttgatacttgattttcaatggttttttgttcatatttgtctttttttttataaccataatagacagggaaaaatgcattttttgtgggagatcttggaaagattgtgaagaaacacagtcaatggcagaatgtagtggctcagataaagccattctacacagtgaagtgcaactctgctccagctgtacttgagattttggcagctcttggaaccggatttgcttgttccagtaaagtaagtatttttcattttttaactaaactgaaacattgaataatttaacttaatgtagttacagcggttagagaacaaatgcaaggaagtttgacttttggatggagtttcaaaataattgtttaaatctccagcctcatgttggaaaattacaagcttttcaagtgcaccaatgaaagggaagtttcagtgtaatgagcctttgaaacgttttatctttattaattgaaaactttaatttgttcctcattcttaggctagagctacttctagaccaaactacagcagatatctaaactctgagatttctttggtgcttctttttagtaatgtttaggccaggagcgatggctaacacctgtaatcccaccactttgagaggctgaggtgagagttgcttgagccaggagtttaggatcagcctgggcaacatagtgagacctcgtctcactttttatttatttatgtttttaaataaaaatattggtttggctaggcacagtgactcatacctgtactcgtagcactttgggaggctgaggcaggaggatcacttgagcagccccagagatggaggctgcattgagctatgattgtgctattgcacttcagcctggaatacaaagagtgagaccctggttttaaaaggggtgggggtaatagtattagaggaagcattaccagaatatgaactgtgacattatgtattctcactttaacctcattttcacagaatgaaatggctttagtgcaagagttgggtgtacctccagaaaacattatttacataagtccttgcaagcaagtgtctcagataaagtatgcagcaaaagttggagtgaatatcctgacatgtgacaatgaaattgaattgaagaaaattgcacgtaatcacccaaatgccaagtaagtataaaattaagtacttgaaaatattatttagcacttagagggttgggtttgtcttttgtctgggaagaaaggagcatttgtggtagggcaatgatgtgtatcataattataacttatttaaagggcctctgtgggaaacactagtaacattttattggctctctacctctgatccttcatgtggaattggaatttaaaaaaataagagcagtttggggatatgatctaataatcttcaaggaatccttgatagttggtgtgataatcaaattagggccatggctgtattttcattatttctataactttgtttgatgtttgataacgttaggaacaggataaactttggtcagcattttttcctgtttagctgaggatttaatctaccctaggccatagttcttgaatggtcatctttactgacagtgtcttcgaagcctagaccatagatttggggggttttgtttttaaggctaatacttttatttttgaacatgaagtttaattttttacttgcatcttcatgtatccagagaattttttttttttcttgattagaaagtttactttttaaaggctgggcgcagtggctcacacctgtaatcccagcactttaggagactgaggctggtggatcatttaaggtcaaggtggcctgcgcctgtaatcccagctacttgggaagctgaggcaagagaatcccttgaacccaggagccggaggctgcagtgagccaagatcgagccactgcactccagcctgggcaacagagcaagactccctcataaaaaggtttacttttttaaaagcaaaaagatttaaggcaaattttatgttttcacaaactggtatatcgagagggcttaagaatatgctaaaagcatattgaattatgcagttgttggcttactttttaatgagttatgtgtgtttccggaaatgaattctatctgtattttctttgtatgtctataattttcatactgactttaatggtgtacttaagatatcctgacagcattgattactctggtaaatctttgtgtggataaaagccttgttgggaaaattctagggaaccacttgtttttctaaaaaatattgttctgtgatcctctgaagacaagctgtgatttgtgatgtttagtatgttagattgtggactggcatcttcttgatggagctgattctaaccttagaataatttttgctttcatcaatcttgtcctctgattatcaaattagggccatagctgtattttcatggccatattattaaccttcttagtttatgtaattattacatccataggaaaacagttacacaaaaagaatttgtatattttcaacttctagcagtttgtaattactcagctcctgaaattaaagaaatttaatcagttttagtcatcttgtcttggttgccatggtttggaaggaaataccaaatagatttgaatcagtagactagaaggctgctgtttaaacacatgaaataattttttaaaaagctttctgggttgggcgcagtagctcatgcctgtaatcccagcacttggggaggccgaggcgggtggatcacttgaagtcaggagtttgagaccagcctggccaacatggtgaaaccctgtctccactaaaaataaaaaaaattagctgggcgtggtggtgcacccttgtatttccagctactttgggaggctgaggcaggagaattgcttgaactcaggaggcactctcatgaggcggaggttgcagtgagctgaaattgcgccactgcactccagcctgggcgacagagtgaaactccatctcagaaaaaaaagaaaaaaaaatttaaaaaatgcttcctatttttttccagttagagacctgctgggatgtaacaatgttagtaggaagaatgtttgcagtaaccatggaggtttagctagttagcacaatcaaagaaatttgaagacatttaaagccctgatatttttattgaaagacaatgatggcattgctattcaatttagtcttcctaggtggatttatggactgatggacttcatttgaaagtaatgtcgtctttcattacagagcccaaccaactcttgatgctacaacagagtcttaacttgctgcattttccaaaatcccatccttgtaagcttcatgctttaggccctaattgctagttttttctctatacaggattgtttttcattaggttcacttgattcatccgtcgctggatttgggagcactggcaacataatcaacacacttcctacaatcttcaggcttcacatgtgctgatgatgatgtaaaccaactctgccccaatcatctccccttctcttagggtcttactacatattgcaacagaagataatattggaggtgaagagggtaacatgaagtttggcactaccctgaagaactgtaggcatctcttggaatgtgctaaggaacttgatgtccaaataattggggttaagtgagtatttgttttaaacttttaaagctgtgaacattttatggtggtaacctgtctttctggttattgatttaagactttatttgcatgaaatctaagatgcaattttttctagtcttttaacacacatgtatgtgttttcttcttagtttaggcaaaacaacttaaatatgccttgtcccttttcatactgttttgtgactgtacctccttccccacaataaaaagaatagcaaataatattttgattcagttagtgcagagtaacttaaatcgccgccttttaaattggatgggaggggatttgctagaattcttaatgaaaataaaagtgtcagacttttttgtccttttaacgattactctgggggacttcaataaattagactttgctttagccttttaattttatatgtgaggaaaccgagactccaagaggttcaaatacttgataatagcctcagaactaattacaggcatagtaacactaagccctagatgttatttgccagcccattgctacttttactaaagtaaaaaggtcttgctttttcaaacactcacaaagatgtattgtgtatctttctttaattttattatttattttttgagatagtatctcattctgtctcccatgctggagtgcagtggcacgatcacggctcactgcagcctcaaactcctggctcaagcaatcttcctgccttggcctcccaaagtggtgggattacagatgtgagcctctgtgcctggccttattgtgtatctttctatactactttgtacataaataggtggtagtcttttaaacaaagaatgggatcatgctgtgccttacacaatagataacattccatgtcagtctagttttaagttttaatttaagtcaacataaaaattaatgaagtactatctcgaatagatgaagggaaaagaagaatggactttacgggtacagttctatggattttagtacttgtgtgaccactgccacaatcaggaagtattctattctgaaactttatcactagtaatactagtgaataatagtaatgctgttactcagcttgattaatgttcatttttatttatttcacaacagacacatttttgtatttggttgggtaaaattgaatgttcagttttatgctcaactgaatgtgatacgatttggtagtgcttgttacttcactatataaacataaaagtgccacatatacataaaagcacaccttattgtataaccgtggaacagtactgataattgacttattgttaagaatttttgtttatagaattacagttttatcttggcattgccttttctataaagctggtactgtaacttttgggtaattagaagctaatgcttctaatactttaaatatttggtaaagttaatggggatacttaatggtatgaatatgatgagtgacagttgaaagaaattacaggaggtgtctctacaactttctgctcctttaaatttagtattctatgtgtttcagatttcatgtttcgagtgcttgcaaagaatctcaagtatatgtacatgctctatctgatgctcgatgtgtgtttgacatggctgtaagttctttcttttaagttattttgatattttattttagccaggatgttaagttaaagactaagtattttctgcctgtgtgaattactctgactacattcaacgtgacacacacctcatttttattatataagcattcagagctctgtacagtcacaccttttaagcaggtaaaggagtgaaatagtaatttacttgcataagtctagaagagagtacaaataaaatcaaagagacagcatacctaacattgaataatctgtttctatcagtaaaaaaccaagaatttgctgtactaaagatcagaacagatatttacctccctaggagggggtttttaaaatgttctcatatccattacaatttggaaaaacatttaaattttttttttccatctctagggagaaattggctttacgatgaacatgttagacattggtggaggattcacgggaactgaatttcaattggaagaggtaaacttctcagtagatgacattaacagaataattgctttctttgttacttaagtggtggatgtgtagagcaggatttttacaatggcagaaactaaaccctataacatctctttaaaattatgttcttagaaaaggaataggcagttagtagacctagctaactttgaaaagtttgatagaattctttgaacattatcataaaacattttctatttcagttgtgtttctgtacttaatggcacaaatgatttaatacttagaaaacttcactactacctttttttttttttttttaaagtcttcctgtgttgcccaggctggagtggagtggcgcaattatagctcactatagcttccaactcctgggctcaagcgatcctcctgcttcaacctcccaagtagctgggacttaaaaaaaaaaaaaaaaaaaaattgagtctttctatattgcccaggctggtctcggaactcctggcctcaaatgatcctcctgtccccaagtgatcctcctgccccagcctcccaaagttttggggttacaggtgtgagccaccactcccagccaactttagcatttaggccagatacacgtcacacctgtttctcaccttcctgctctgccctccttccttctgtgatatctagaaactatgatttgaaaattttacttctgtttttagtgatcaagtatagaaaaatgacttaaaaaaaaacaaaaacaaataccagtgggtatttgttattagagctatttttaaaattaatgccattctgatagttacaaaataaactgacttgaaagcatgcctaattaattgcttggagataactacaaagttgctaaggttcatgatctgtgattggaactgcttcattttcctagaaaattttgttaatatcaatctctgaatattgcgaattatctaatccaatattgaaaccatactgtggcatttaaaatccataggttaatcatgttatcagccctctgttggatatctactttcctgaaggatctggtgttaagataatttcagaacccggaagctactatgtgtcttctgcatttacactcgcagttaatatcatagcaaagaaagttgttgaaaatgataaatttccctctggaggtaagttataaagtttatagttttatttttgttggtagataattgcagttagtctggaaatgagcagatggtcatgaagttgtaggtggttttcagcttttaaaaatcttaaaactggccgggcgtggtggctcacgcctataatcccagcactttgggaggcagagccaggtggatcacaaggtcaggagttcgagaccagcctggccaatatggtggaaaccccatctctactaaaaatataaaacttagctggtcatggtggcgcacacctgtaatcccggctactcgggaggctgaggcacaaaaatcgcttgaatccgggaggcagaggttgcagtgagccaagattgtgccactggactccagcctgggtgacaagagtgagactccgtctcaaaaaaaaaaaaaatcttaaagccaaggtcccattccgtaagacttccaaatccaactttcttggtactgatgtttagatacgtgtgttttagataaatgtctagcagatgattaattctcatccattattcagaactacttttattgtgatcttttctgttctccttacctcacatgggtcagagggaggctctgttttcacactttctcacataagggccacaaagagtccctgtaatctaactcaagtcaaaccaacaaaaaggcactagacagtaccatcttgttggctaatattgtaggaaatactgttttacattaagaagacttttttttttggctgtggactaatccacagcccataggaaagttgtccatttccccaatattgtcttttttttacagctttgtttttccttgcttagaatccagtccaggaccacattgaattcgtcatttctccttgaatctagataaagtttctcagctcattactttgtattttttaactttgacatttttgaagagtagaaatttcatagctgatccttagtttgggtttgttggatgattcctcatgattaattttttaggctatgcatttggggcatgaagtccactaaagtgttactctgttcttctcacttcatcatgttaggaggcacatgatagtggccaattctaatattgatgatgctaggtgttttttcttgttgttttgtcgcccaggctggagtacagtggctccatcagggctcattgcagtctccacctcccggcttaagcagtcctcctaccttcaacctcttgcatagctgggactacaggcatatgccaccatgcctggctaggtttttttctatttttattttgtagagggtttcgtcgtgtttctcaggctggtctcaaactcctgagctcaagtgatccacctgcctcagcctcccaaagtgctgggattacaaacttgagtcaatacacctggccttatgattttttttttttttttttttttgagactgttttgctcttgttgcccaagctggaatgcagtggcatgatctcagctcactgcaacctccacctcccaggttcaagcaattctcctgcctcagcctcccaagtagctgggattacaggtgcgtgccaccacacccggctagatttttttttttttttttaatttaaatgctagttttgagcacttgtttaaggttgtgtcaggcaggtttctttccattgtgaagtggcaagtttaaattagagactctgtaaactacctgtttttaaccagattttcaccccattagttttagcatctattgttgaatttctaacttcattattgtttatattagttggcctttacttaaagaatatcttttccttccctcctgtttatgagtttagactcatagatctttattcaataggtttatataatctgttactcttatttttcccttgacatgtctccattattctcagagcaattcgttattttttggtatatcaagatgttccaggctcattttaaccttttaaatgagcctcagtcccgtaatcagttgtttctccagggagccttggtttgttttcgtggagagtgatattttaaaaacctgaaaattagtattggttgtgctaagagtagagtagctgtgagtagaatgggcgttttcaaaggtttcaaagtgaggtgagaggatctattgatccctgaagctcgaggctgcggtgacctgtgatcacgctactgcactgtagcctgagtaacagtgaggccctgtctctacacacacacacagattcccactggcagtgtatacatgtgagctctggttgctcctcctgctctctgatgttttgtagtgtcagtctttaagttgatccagtcagtaggttggtggtaatggtagtccattgtggttttaatttgcatttacctgatgactaacgatgtagagcactgttttctttttctttttcttttcttttttttttttgtttttgagacagggtttttgctctgttatccaggctggaatgagtggcacaatctcaacttttatttttaattctacattcaaacacatatttaactccttgtgcatatattgccttccccttgctgatttgaaaagaaactaaaaagtgtttttccttgctgcttcatttcctctttttttgaaacagcaactatcagaaagtcttctagaatcttaatacttttttctttttttttttttttttttgagacagggtctcattctgtcacccaggctggagtgcagtagcacagccttggctcactgtctcactgtcttactgtaacctccgcctcccggattcaagggattctagtgcctcagcctcccaagtagctgtgattacaggcatgcaccaccacacctggctaatttttgtatttttagtagagatggagttttgccatgttgttcaggctgatcttgaactcctgacctcaagtgatcctcccacctcagcctcacaaagtgctaggattacaggtatgagccaccgcatccagccctagagcactcttttcttgtgctcattggttatcttcctttgtgaaatgcgtgctctatgtgtctttttaagtttaaatagtttgagaaatgtgaaaattacattaatttgtttcatatgagccattttattaaaaattcattttcccctttcctgttttattccagtagaaaaaaccggaagtgatgaaccagccttcatgtattatatgaatgatggtgtttatggttcttttgcaagtaaactgtctgaggacttaaataccattccagaggttcacaaggcaagttttatcagaaatatcaaaacctatttggcattttataagctgagctgttatttttaagatgcttcccagttttttgaaaaacatcttagaatggggaaaaatgtcctttttgaggatttgcgttcttctggtggagtcccttttttagtttgttgcctttacgtggagttaatggtaccatcgaggcttaagtagcaccctgcccttggctctaggccttttttccatgagcacattggatctgagacagtgaaaatacttatttactcacaaattcaatacctgcgtaagtatgagtaaaaattaaattgtaaaacttttcctacagaaatacaaggaagatgagcctctgtttacaagcagcctttggggtccatcctgtgatgagcttgatcaaattgtggaaagctgtcttcttcctgagctgaatgtgggagattggcttatctttgataacatgggagcagattctttccatgaaccatctgcttttaatgattttcagaggccagccatttattacatgatgtcattcagtgattggtaaggtgattttattttaaggcagatgggatatttgaactgtccttttagacttgctaataagttttaattctgtaatgaatgctaaacttagaagtacagtcatgtattgcctatacagtgtagttacttaatgatgggaatttgttctgacaaatacaacattaggcgattgtatgatggcataacctactacataatctaggccatgtggtatattgatcctaggctacaaacctgtacagcatgttactctgtttaatactgttggcagttgtaatgcagtggcgtttgtgtatcttaagttacttaaacatagaaaggataatacattgtgctatgacatgattgctgcgatgtaacgaggtgataggactttttaaacttcactgtaattttacggtataccacagtcatatgtgtagtctttgaccaaaacatcactctgcagtgcatgactatcatagaaaaatgatgatcttttaaaggtttttggaagactagttataatcctgttttattatccatttgttatgtgattacgtggtctttgggcttaaactgagaatttagctgattttttttctacgtaggtatgagatgcaagatgctggaattacttcagactcaatgatgaagaacttcttctttgtgccttcttgcattcagctgagccaagaagacagcttttccgctgaagcttaaacaggcattaacgcttctttagatctgaagttgcaggttaagcttgtctggtcaacattccagtgtggaaaaataatttaaacaatcttattctcttaattcttttggcaacaaaaactattagtaatagctatttgggaccagacaaaatcagctttcatctataattcattggggataatgggagatttagataatgtatccagatttaaacctaccagtttgtcctaccccttaagcgtttaaaataaaatatgcaacaaaatggatgacttagtggagatggaagcccattaattgggttccccattaaatcgtttacatacaagaacacagtttttatactaaggatttgtgtttaaagtcttgtaaagttcatgtctttcacccagatatatcaaatgttagaagaccagtgtgacttcattagataacgtttagtgtatttagaatgtgtaaatttgtgctttgaactgtagtttaataaatgtaaaattgcatcatagtatttgttgacctaatgtaacccttgtatgattgcaataaaattttgtgtagattttactgttttttcaggctaaaactttgggaaaggggctagctagcaaaggtagttttgaaatagatgtgtatatggactgttttgaagggtttttttctttatagcccagttaagttttgtttggctcggtgcatttttcatttatttaattagtaatttaagtaaagtgtttggtaaatcattgtgaagttcagattcattatggagagttgatgtgcagtaagcatgatgtttaacaattttaacaccaaaaatgttaatcctgcataaatcaactgtaataataaataggtgtttctgtatagatagaatgcatagagtaccttagtaaatctttgaatcacaatcttttggctgaaatggaagattctgttaaatactttgaataaacttggggggagggaaataaaattgcagaaaactgcagagcactaaaacttaaagaagggctacatctttatccagaaacctgttgctcttttgcacggaatgtttaaattcagagttgggatgggggttggggtgaagcacacttattatcttcagttgcagtgatttcaaatttaggattttttgttgttggtttgaactgtccccttagtttcttgttatttccaatttgttctgcttagtcattacttttaattcttttcttactaaaattttatggtggttgggggaagggagttagcatcactaacctgacagttgttgccaggaatttgctttgtttactgctagtatattagaaatcctagatctcagaatcacaatagtaataaacaacaggggtcattttttcctaacttactctgtgttcaggtgtggaatttctgtctcccaagaggaaatgtgacttcactttggtgccaatggacagaaaattctacctgtgctacataggagaagtttggaatgcacttaatagctggtttttacaccttgatttcgaggtggaaagaaattgatcatgaatctctaataaatttaaatctcttaaaccagtaggtgcttaatattttttgatttgattaatgcccatttaaatctcatgggttctattaaaaatatatatatatagggccccaatccattgccatcaaattgcccttggacttttccaaggtatattatggggttttatgcaaaattccaagctaccatgtaactttttttaaccatttaacaaggagggggaactgtttcctaccttctttacatgttgtgcattgttgtggtccagaaatgccaaacctttttaaagatggtgcaactttgagtccttggcttgactatacaggccttgaacttcatggcatatcaactttgccatatctgcaggagagctgttctataagaaatagctcagagttgcaaatatcacatgtgaatgatacggtaacttttaagaaatgtctgtattgtatttgaagactgtttgccataaatctgaaatttgaacctatgtatttcaatttggtatgctaaaaagttctgaattaatgtaaagttttttgttataatattgtaatctcagttcaaaagttaactgcaaatataaaacccaatgatttctatatagtaaattgaactgtaaaggtaacttgtgtgtgattctgaatacatagataaatgtttttattcctcatgttttactttggcttctatctgaaatagaggtaaaattttacatatcagcttta

SEQ ID NO: 4 - protein, 448 aa, Artificial Sequence
  Synthetic sequence
MKGFIDDANYSVGLLDEGTNLGNVIDNYVYEHTLTGKNAFFVGDLGKIVKKHSQWQNVVAQIKPFYTVKCNSAPAVLEILAALGTGFACSSKNEMALVQELGVPPENIIYISPCKQVSQIKYAAKVGVNILTCDNEIELKKIARNHPNAKVLLHIATEDNIGGEEGNMKFGTTLKNCRHLLECAKELDVQIIGVKFHVSSACKESQVYVHALSDARCVFDMAGEIGFTMNMLDIGGGFTGTEFQLEEVNHVISPLLDIYFPEGSGVKIISEPGSYYVSSAFTLAVNIIAKKVVENDKFPSGVEKTGSDEPAFMYYMNDGVYGSFASKLSEDLNTIPEVHKKYKEDEPLFTSSLWGPSCDELDQIVESCLLPELNVGDWLIFDNMGADSFHEPSAFNDFQRPAIYYMMSFSDWYEMQDAGITSDSMMKNFFFVPSCIQLSQEDSFSAEA

SEQ ID NO: 5 - DNA, 20 bp, Artificial Sequence
  Synthetic sequence
uuaagcuucagcggaaaagc

SEQ ID NO: 6 - DNA, 20 bp, Artificial Sequence
  Synthetic sequence
  misc_feature (1)..(20): 2′-O-Me sugar modification
uuaagcuucagcggaaaagc

SEQ ID NO: 7 - DNA, 20 bp, Artificial Sequence
  Synthetic sequence
  misc_feature (1)..(20): 2′-O-Me sugar modification
  misc_binding (1)..(20): Phosphorothioate bond
uuaagcuucagcggaaaagc

SEQ ID NO: 8 - DNA, 20 bp, Artificial Sequence
  Synthetic sequence
  misc_feature (1)..(20): 2′-O-Me sugar modification
  misc_binding (1)..(6): Phosphorothioate bond
uuaagcuucagcggaaaagc

SEQ ID NO: 9 - DNA, 20 bp, Artificial Sequence
  Synthetic sequence
  misc_feature (1)..(20): 2′-O-Me sugar modification
  misc_binding (15)..(20): Phosphorothioate bond
uuaagcuucagcggaaaagc

SEQ ID NO: 10 - DNA, 37 bp, Artificial Sequence
  Synthetic sequence
cgggatccattcattttcccctttcctgttttattcc

SEQ ID NO: 11 - DNA, 31 bp, Artificial Sequence
  Synthetic sequence
cggaattctccagcatcttgcatctcatacc

SEQ ID NO: 12 - DNA, 32 bp, Artificial Sequence
  Synthetic sequence
cggaattcgcttaaggggtaggacaaactggt

SEQ ID NO: 13 - DNA, 44 bp, Artificial Sequence
  Synthetic sequence
cgggatccgcaagttttatcagaaatatcaaaacctatttggca

SEQ ID NO: 14 - DNA, 39 bp, Artificial Sequence
  Synthetic sequence
cgggatccaaatacaaggaagatgagcctctgtttacaa

SEQ ID NO: 15 - DNA, 32 bp, Artificial Sequence
  Synthetic sequence
cggaattcttaagcttcagcggaaaagctgtc

SEQ ID NO: 16 - DNA, 30 bp, Artificial Sequence
  Synthetic sequence
ctgagccgaattcaatcgatggccgccatg

SEQ ID NO: 17 - DNA, 35 bp, Artificial Sequence
  Synthetic sequence
gaattcggctcagctgaatgcaagaaggcacaaag

SEQ ID NO: 18 - DNA, 36 bp, Artificial Sequence
  Synthetic sequence
gaagacagctgaagcttaagaattcaatcgatggcc

SEQ ID NO: 19 - DNA, 33 bp, Artificial Sequence
  Synthetic sequence
ttcagctgtcttcttggctcagctgaatgcaag

SEQ ID NO: 20 - DNA, 41 bp, Artificial Sequence
  Synthetic sequence
ctcattcagtgcagaagcttaagaattcaatcgatggccgc

SEQ ID NO: 21 - DNA, 40 bp, Artificial Sequence
  Synthetic sequence
tgcactgaatgagtcttcttggctcagctgaatgcaagaa

SEQ ID NO: 22 - DNA, 32 bp, Artificial Sequence
  Synthetic sequence
cgggatccatcatgcacctctgcgctatatcg

SEQ ID NO: 23 - DNA, 35 bp, Artificial Sequence
  Synthetic sequence
cggaattcagaacccgatcaaacgcaaatgttacc

SEQ ID NO: 24 - DNA, 24 bp, Artificial Sequence
  Synthetic sequence
gctgaagctggaagcaagaaagtg

SEQ ID NO: 25 - DNA, 21 bp, Artificial Sequence
  Synthetic sequence
cagggccttctttggacagga

SEQ ID NO: 26 - DNA, 26 bp, Artificial Sequence
  Synthetic sequence
attgatgatgcaaactactccgttgg

SEQ ID NO: 27 - DNA, 22 bp, Artificial Sequence
  Synthetic sequence
ctggaggtacacccaactcttg

SEQ ID NO: 28 - DNA, 20 bp, Artificial Sequence
  Synthetic sequence
accctgaagtaccccatcga

SEQ ID NO: 29 - DNA, 23 bp, Artificial Sequence
  Synthetic sequence
ctcaaacatgatctgggtcatct

SEQ ID NO: 30 - DNA, 29 bp, Artificial Sequence
  Synthetic sequence
gcatttacactcgcagttaatatcatagc

SEQ ID NO: 31 - DNA, 25 bp, Artificial Sequence
  Synthetic sequence
aatgcaagaaggcacaaagaagaag

SEQ ID NO: 32 - DNA, 24 bp, Artificial Sequence
  Synthetic sequence
cggattccccgtgccaagagtgac

SEQ ID NO: 33 - DNA, 20 bp, Artificial Sequence
  Synthetic sequence
ggccatggcggccatcgatt

SEQ ID NO: 34 - DNA, 28 bp, Artificial Sequence
  Synthetic sequence
tacaaggaagatgagcctctgtttacaa

SEQ ID NO: 35 - DNA, 23 bp, Artificial Sequence
  Synthetic sequence
tccagcatcttgcatctcatacc

SEQ ID NO: 36 - DNA, 27 bp, Artificial Sequence
  Synthetic sequence
cctacagaaatacaaggaagatgagcc

SEQ ID NO: 37 - DNA, 30 bp, Artificial Sequence
  Synthetic sequence
taaaatcaccttaccaatcactgaatgaca

SEQ ID NO: 38 - DNA, 86 bp, Artificial Sequence
  Synthetic sequence
ugagcuugaucaaauuguggaaagcugucuucuuccugagcuagcugagccaagaagacagcuuuuccgcugaagcuuaaacaggc

SEQ ID NO: 39 - DNA, 21 bp, Artificial Sequence
  Synthetic sequence
caagaagacagcuuuuccgcu

SEQ ID NO: 40 - DNA, 20 bp, Artificial Sequence
  Synthetic sequence
uguggaaagcugucuucuuc

SEQ ID NO: 41 - DNA, 20 bp, Artificial Sequence
  Synthetic sequence
  misc_feature (1)..(20): 2′-O-Me sugar modification
ccacaauuugaucaagcuca

SEQ ID NO: 42 - DNA, 20 bp, Artificial Sequence
  Synthetic sequence
  misc_feature (1)..(20): 2′-O-Me sugar modification
  misc_binding (1)..(20): Phosphorothioate bond
ccacaauuugaucaagcuca

SEQ ID NO: 43 - DNA, 20 bp, Artificial Sequence
  Synthetic sequence
  misc_feature (1)..(20): 2′-O-Me sugar modification
  misc_binding (15)..(20): Phosphorothioate bond
ccacaauuugaucaagcuca

SEQ ID NO: 44 - DNA, 20 bp, Artificial Sequence
  Synthetic sequence
  misc_feature (1)..(20): 2′-O-Me sugar modification
  misc_binding (1)..(6): Phosphorothioate bond
ccacaauuugaucaagcuca

SEQ ID NO: 45 - DNA, 20 bp, Artificial Sequence
  Synthetic sequence
  misc_feature (1)..(20): 2′-O-Me sugar modification
gcugucuucuuggcucagcu

SEQ ID NO: 46 - DNA, 20 bp, Artificial Sequence
  Synthetic sequence
  misc_feature (1)..(20): 2′-O-Me sugar modification
cuucagcggaaaagcugucu

SEQ ID NO: 47 - DNA, 25 bp, Artificial Sequence
  Synthetic sequence
  misc_feature (1)..(25): 2′-O-Me sugar modification
gcuuuccacaauuugaucaagcuca

SEQ ID NO: 48 - DNA, 20 bp, Artificial Sequence
  Synthetic sequence
  misc_feature (1)..(20): 2′-O-Me sugar modification
gcuuuccacaauuugaucaa

SEQ ID NO: 49 - DNA, 20 bp, Artificial Sequence
  Synthetic sequence
  misc_feature (1)..(20): 2′-O-Me sugar modification
cagcucaggaagaagacagc

SEQ ID NO: 50 - DNA, 10 bp, Artificial Sequence
  Synthetic sequence
  misc_feature (1)..(1): NH2-Lys modified
  misc_feature (3)..(3): m represents thio-pseudoisocytosine
  misc_feature (6)..(6): m represents thio-pseudoisocytosine
  misc_feature (8)..(8): n represents guanidine-modified 5-methyl cytosine
  misc_feature (10)..(10): m represents thio-pseudoisocytosine
  misc_feature (10)..(10): CONH2 modified
ttmttmtntm

SEQ ID NO: 51 - DNA, 12 bp, Artificial Sequence
  Synthetic sequence
  misc_feature (1)..(1): NH2-Lys modified
  misc_feature (12)..(12): CONH2 modified
agcggaaaagct

SEQ ID NO: 52 - DNA, 20 bp, Artificial Sequence
  Synthetic sequence
  misc_feature (1)..(20): 2′-O-Me sugar modification
  misc_binding (15)..(20): Phosphorothioate bond
cguguguucuacgcucuggu